(19) United States
(12) Patent Application Publication
(10) Pub. No.: US 2003/0054347 A1
(43) Pub. Date: Mar. 20, 2003

(54) Latrophilin Polynucleotides

(75) Inventors: Julia E. Richards, Ann Arbor, MI (US); Frank W. Rozsa, Chelsea, MI (US)

Correspondence Address: MEDLEN & CARROLL, LLP, 101 HOWARD STREET, SUITE 350, SAN FRANCISCO, CA, 94105, US

(73) Assignee: The Regents of the University of Michigan

(21) Appl. No.: 09/844,653

(22) Filed: Apr. 27, 2001

(51) Int. Cl. C12Q 1/68; C07H 21/04; C12P 21/02; C12N 5/06

(52) U.S. Cl. 435/6; 435/69.1; 435/320.1; 435/325; 530/350; 536/23.2

(57) ABSTRACT

The present invention relates to Latrophilin (LPH) polynucleotides and polypeptides, and to identifying and treating subjects at risk for eye disease. Specifically, the present invention provides novel human LPH3 and LPH1 polynucleotides and LPH3 polypeptides, assays for detecting variations in LPH polynucleotides associated with eye disease, and methods and compositions for treating eye disease.

The present application was funded in part with government support under grant number C033662, from the National Eye Institute at the National Institutes of Health. The government has certain rights in this invention.

### FIELD OF THE INVENTION

The present invention relates to Latrophilin (LPH) polynucleotides and polypeptides, and to identifying and treating subjects at risk for eye disease. Specifically, the present invention provides novel human LPH3 and LPH1 polynucleotides and LPH3 polypeptides, assays for detecting variations in LPH polynucleotides associated with eye disease, and methods and compositions for treating eye disease.

### BACKGROUND OF THE INVENTION

“Glaucomas” are a group of debilitating eye diseases that are among the leading causes of preventable blindness in the United States and other developed nations. Primary open-angle glaucoma (“POAG”) is the most common form of glaucoma in Caucasians and African Americans, but also occurs in the Asian population in which angle-closure glaucoma is more common. This disease is characterized by death of retinal ganglion cells with accompanying loss of visual field and cupping of the optic nerve which is evident upon ophthalmologic examination. POAG is commonly associated with elevated intraocular pressure (“IOP”), although open-angle forms of glaucoma may also be found in which elevated IOP has not been observed and individuals with elevated IOP do not all go on to develop glaucoma. Elevated IOP most often results from reduction in outflow of fluid from the eye via a tissue type called the trabecular meshwork. The trabecular meshwork is the area in which level of IOP appears to be regulated and where reduction of outflow and subsequent elevation of IOP appears to occur in POAG. Some aspects of POAG pathology may also be relevant to other forms of glaucoma including other forms of open-angle glaucoma. The disease is estimated to affect between 0.4% and 3.3% of all adults over 40 years old. Moreover, the prevalence of the disease rises with age to over 6% of those 75 years or older.

Primary Open Angle Glaucoma (POAG) is characterized by atrophy of the optic nerve resulting in visual field loss and eventual blindness. POAG has been divided into two major groups, based on age of onset and differences in clinical presentation.

Juvenile-onset Primary Open Angle Glaucoma usually manifests in late childhood or early adulthood. The progression of Juvenile-onset POAG is rapid and severe with high intraocular pressure that is poorly responsive to medical treatment, and is such that it usually requires ocular surgery. Adult- or late-onset Primary Open Angle Glaucoma is the most common type of glaucoma. It is milder and develops more gradually than juvenile-onset Primary Open Angle Glaucoma, with variable onset usually after the age of 40. Late-onset POAG is associated with slight to moderate elevation of intraocular pressure, and often responds satisfactorily to regularly monitored medical treatment. However, because the disease progresses gradually and painlessly, it may not be detected until a late stage when irreversible damage to the optic nerve has already occurred.

Because increased IOP is a readily measurable characteristic of glaucoma, the diagnosis of the disease is largely screened for by measuring intraocular pressure (tonometry). Unfortunately, because glaucomatous and normal pressure ranges overlap, such methods are of limited value unless multiple readings are obtained. For this reason, additional methods, such as direct examination of the optic disk and determination of the extent of a patient's visual field loss are often conducted to improve the accuracy of diagnosis.

Because of the insidious nature of glaucoma, a need remains for a better and earlier means to diagnose or predict the likelihood of development of glaucoma, so that preventative or palliative measures can be taken before significant damage to the optical nerve occurs. As such, what is needed are improved, more accurate methods for diagnosing glaucoma.

### SUMMARY OF THE INVENTION

The present invention relates to Latrophilin (LPH) polynucleotides and polypeptides, and to identifying and treating subjects at risk for eye disease. Specifically, the present invention provides novel human LPH3 and LPH1 polynucleotides and LPH3 polypeptides, assays for detecting polymorphisms in LPH polynucleotides associated with eye disease, such as glaucoma and related diseases, and methods and compositions for treating eye disease.

The present invention provides human LPH3 and LPH1 nucleic acids and proteins. Thus, in some embodiments, the present invention provides an isolated and purified nucleic acid sequence comprising the cDNA sequence of human LPH3 as shown in SEQ ID NO:1, or fragments or variants thereof. In other embodiments, the present invention provides an isolated and purified nucleic acid sequence comprising the MRNA sequence of human LPH3 (e.g. replace the T's in SEQ ID NO:1 or 2 with U's), or fragements or variants thereof. In particular embodiments, the present invention provides an isolated and purified nucleic acid sequence comprising the cDNA sequence of human LPH3, further comprising 5′ and/or 3′ untranslated regions as shown in SEQ ID NO:2, or fragments or variants thereof. In some embodiments, the present invention provides isolated and purified nucleic acid sequences comprising the human gene sequence of LPH3 (as shown in SEQ ID NO:5), or fragments or variants thereof. In other embodiments, the present invention provides isolated and purified nucleic acid sequences comprising the human gene sequence for LPH1 (shown in SEQ ID NO:32), or fragments or variants thereof.

In some embodiments, the present invention provides an isolated and purified nucleic acid comprising a sequence encoding a human LPH3 peptide as shown in SEQ ID NO:3, or fragments or variants thereof. In some embodiments, the nucleic acid sequences of the present invention are operably linked to a heterologous promoter. In some embodiments, the nucleic acid sequence is contained within a vector. In further embodiments, the vector is within a host cell.

In other embodiments, the present invention provides an isolated and purified nucleic acid sequence that hybridizes under conditions of high, medium or low stringency to a nucleic acid selected from the group consisting of SEQ ID NO:1, 2, 5, and 32. In some embodiments, the nucleic acid sequence encodes a protein that is a receptor for the TIGR/MYOC protein. In other embodiments, the present invention provides a vector comprising the nucleic acid sequence. In still other embodiments, the vector is within a host cell. In some embodiments, the host cell is located in an organism selected from the group consisting of a plant and an animal.

In further embodiments, the nucleic acid molecule is a human LPH3 nucleic acid that is at least about 70%, preferably about 80%, more preferably about 85%, and even more preferably at least about 90% or 95% (e.g. 96%, 97%, 98%, and 99%) homologous to the nucleic acid shown as SEQ ID NOS:1, 2 or 5, or to the complement of the nucleic acid shown as SEQ ID NOS:1, 2, or 5. In certain embodiments, the present invention provides nucleic acid molecules comprising at least 50, at least 100, at least 200, or at least 250 consecutive nucleotides of SEQ ID NO:1 or 2 (e.g. fragments of LPH3 to be used as a control sequence in the diagnostic methods described below).

In some embodiments, the present invention provides probes and primers comprising substantially purified oligonucleotides, which correspond to a region of nucleotide sequence which hybridizes to at least about 6, at least about 10, at least about 15, at least about 20, or preferably at least about 25 consecutive nucleotides of the sequences set forth as SEQ ID NOS:1, 2, 5, and 32, or complements of the sequences set forth as SEQ ID NOS:1, 2, 5, and 32, or naturally occurring mutants or allelic variants thereof. In preferred embodiments, the probes and/or primers further include a label group attached thereto, which is capable of being detected. In some embodiments, the primers and probes are complementary to exon portions of SEQ ID NOS:5 and 32 (i.e. regions in bold and uppercase in FIGS. 7 and 9, e.g. SEQ ID NOS:58 and 59). In preferred embodiments, the primers and probes are complementary to intronic portions of SEQ ID NOS:5 and 32 (i.e. regions not labelled as exons in FIGS. 7 and FIG. 9, e.g. SEQ ID NOS:56 and 67). In particulary preferred embodiments, the primers are complementary to intronic regions immediately surrounding exon regions (see FIG. 7 and 9), such that, for example, the exon regions (e.g. Exon 4 of human LPH3 or LPH1) may be PCR amplified (e.g. prior to detecting variations in the amplified regions).

In yet other embodiments the present invention provides a protein encoded by a nucleic acid selected from the group consisting of SEQ ID NOs:1 and 2, and fragments, and variants thereof that are at least 80% identical to SEQ ID NOs:1 and 2. In some embodiments, the protein has at least one activity of LPH3 (SEQ ID NO:3). In some embodiments, the activity is binding the TIGR peptide. In some embodiments, the protein is at least 90% identical to SEQ ID NO:3, in other embodiments, the protein is at least 95% identical (e.g. 96%, 97%, 98%, and 99%) to SEQ ID NO:3. In preferred embodiments the protein is at least 98% identical to SEQ ID NO:3.

In still further embodiments, the present invention provides a method for producing variants of human LPH3 comprising: providing a nucleic acid sequence selected from the group consisting of SEQ ID NOs:1 and 2, and complements thereof, and mutagenizing the nucleic acid sequence; and screening the resulting variants for LPH3 activity (e.g. binding the TIGR peptide in a cellular or biochemical assay).

In other embodiments, the present invention provides a composition comprising a nucleic acid that inhibits the binding of at least a portion of a nucleic acid selected from the group consisting of SEQ ID NOs:1, 2 and 5 to their complementary sequences. In certain embodiments, the present invention provides anti-sense molecules that inhibit the production of an LPH3 peptide. In certain embodiments, the antisense molecules are capable of binding to LPH3 MRNA transcripts.

In further embodiments, the present invention provides compositions comprising an isolated and purified nucleic acid, wherein the nucleic acid comprises a sequence that shares at least 96% identity with SEQ ID NO:1 (the cDNA sequence of human LPH3) or the complement of SEQ ID NO:1, or fragments of SEQ ID NO:1, or fragments of the complement of SEQ ID NO:1. In preferred embodiments, the sequence is human or derived from a human sequence (e.g. by introducing changes, deletion, etc. into the human LPH3 cDNA sequence shown in SEQ ID NO:1). In other embodiments, the present invention provides compositions, wherein the sequence shares at least 97%, preferably 98%, and more preferably 99% identity with SEQ ID NO:1 or the complement of SEQ ID NO:1. In certain embodiments, the sequence is operably linked to a heterologous promoter. In some embodiments, the sequence is contained within a vector. In some embodiments, the sequence is contained within a mutation detecting device (e.g. a microchip, DNA chip, or a molded plastic cartridge). In particular embodiments, the sequence is contained within a vector. In further embodiments, the present invention comprises a computer readable medium encoding this sequence (e.g. SEQ ID NO:1).

In some embodiments, the present invention provides compositions comprising an isolated and purified nucleic acid, wherein the nucleic acid comprises a sequence encoding a peptide that shares at least 96% identity with SEQ ID NO:3, or fragments of SEQ ID NO:3. In preferred embodiments, the sequence is human or derived from a human sequence (e.g., by introducing changes, deletion, etc. into the human LPH3 peptide sequence shown in SEQ ID NO:3). In certain embodiments, the peptide shares at least 97%, preferably 98%, and more prefereably 99% identity with SEQ ID NO:3. In some embodiments, the sequence is operably linked to a heterologous promoter. In other embodiments, the sequence is contained within a vector or a mutation detecting device. In particular embodiments, the present invention provides a computer readable medium encoding a representation of the sequence.

In some embodiments, the present invention provides compositions comprising an isolated and purified peptide, wherein the peptide comprises an amino acid sequence that shares at least 96% identity with SEQ ID NO:3. In preferred embodiments, the sequence is human or derived from a human sequence (e.g., by introducing changes, deletion, etc. into the human LPH3 peptide sequence shown in SEQ ID NO:3). In certain embodiments, the amino acid sequence shares at least 97%, preferably 98%, and more preferably 99% identity with SEQ ID NO:3. In some embodiments, the present invention provides a computer readable medium encoding a representation of the sequence (e.g., SEQ ID NO:3).

In some embodiments, the present invention provide a system comprising a computer readable medium, wherein the computer readable medium encodes a representation of a nucleic acid sequence, wherein the nucleic acid sequence comprises SEQ ID NO:1, SEQ ID NO2, SEQ ID NO:5, or SEQ ID NO:32. In other embodiments, the present invention provides a system comprising a computer readable medium, wherein the computer readable medium encodes a representation of an amino acid sequence, wherein the amino acid sequence comprises SEQ ID NO:3. In further embodiments, the systems of the present invention comprises a processor.

In further embodiments, the present invention provides compositions comprising an isolated and purified nucleic acid, wherein the nucleic acid comprises at least 400 consecutive bases from SEQ ID NO:5 or SEQ ID NO:32. In other embodiments, the present invention provides compositions comprising an isolated and purified nucleic acid, wherein the nucleic acid comprises at least 500, or at least 750, or at least 1000 consecutive bases from SEQ ID NO:5 or SEQ ID NO:32.

The present invention also provides a method for detection of a polynucleotide encoding human LPH3 or LPH1 proteins in a biological sample suspected of containing a polynucleotide encoding human LPH3. The method includes hybridizing the polynucleotide sequence selected from the group consisting of SEQ ID NOs:1, 2, 5 and 32, and variants, fragments, or complements thereof that are at least 90% identical (e.g. 94%, 96%, and 98%) to SEQ ID NOs:1, 2, 5 and 32 to the nucleic acid of the biological sample to produce a hybridization complex. In some embodiments, the method further includes the step of detecting the hybridization complex, wherein the presence of the hybridization complex indicates the presence of a polynucleotide encoding human LPH3 (e.g. SEQ ID NO:3) in the biological sample. In some embodiments, the amount of LPH3 nucleic acid is quantitated. In some embodiments, the nucleic acid employed is complementary to an intronic region of SEQ ID NO:5 or SEQ ID NO:32. In some embodiments, prior to hybridization, the nucleic acid of the biological sample is amplified (e.g. by PCR).

The present invention further provides a method for screening compounds for the ability to modulate LPH activity, comprising: providing: a first polypeptide sequence comprising at least a portion of an LPH peptide; ii) a second polypeptide sequence comprising at least a portion of a protein known to interact with the LPH peptide; and iii) one or more candidate (test) compounds; combining in any order, the first polypeptide sequence comprising at least a portion of an LPH peptide, the second polypeptide sequence comprising at least a portion of a protein known to interact with the LPH peptide, and one or more candidate compounds under conditions such that the first polypeptide sequence, the second polypeptide sequence, and the test compound are capable of interacting; and detecting the presence or absence of an interaction between the polypeptide sequence comprising at least a portion of an LPH peptide and the polypeptide sequence comprising at least a portion of a protein known to interact with the LPH peptide. In some embodiments, the first polypeptide sequence is selected from peptide comprising SEQ ID NO:3, or fragments, or variants thereof, or the peptide encoded by SEQ ID NOS:10 and 11 (i.e. the olfactomedin domain of human LPH3), or fragments, or variants thereof. In some embodiments, the second polypeptide is a TIGR peptide (e.g., human TIGR peptide shown in SEQ ID NO:173), or fragments thereof (e.g. amino acids 246-502 of SEQ ID NO:173, which is an olfactomedin domain).

The present invention also provides methods of identifying individuals suffering from eye disease or at risk for developing eye disease. In some embodiments, the present invention provides methods for determining the risk of eye disease comprising: a) providing nucleic acid from a subject (e.g. eye patient), wherein the nucleic acid comprises an LPH gene; and b) detecting the presence or absence of at least one variation in the LPH gene. In other embodiments, the methods further comprises step c) providing a prognosis or diagnosis to the subject based on the presence or absence of the variation. In particular embodiments, the variation is a mutation. In other embodiments, the variation is a polymorphism (e.g. a single nucleotide polymorphism (SNP)). In certain embodiments, the variation is located within the olfactomedin domain of the LPH peptide (e.g. SEQ ID NOS:10 and 11 of SEQ of human LPH3). In some embodiments, the LPH gene is selected from LPH1, LPH2 and LPH3 (e.g. from a mammal, such as dog, cat, cow or human). In preferred embodiments, the LPH gene is selected from human LPH1, human LPH2, and human LPH3. In further embodiments, the LPH gene is a human LPH3 sequence, and the variation prevents the peptide encoded by the human LPH3 sequence from binding the human TIGR peptide or increases binding affinity between LPH3 peptides and TIGR peptides.

In certain embodiments, the present invention provides kits comprising, a least a portion of an LPH gene, and detection means (e.g. probes, primers, other oligos). In some embodiments, the kit further comprises instructions for use. In particular embodiments, the various components are contained in separate containers or the same container. In some embodiments, the kits further comprise a control LPH gene (i.e. a portion of an LPH gene known to contain or not contain a variation being screened for). In particular embodiments, the present invention provides a kit for determining if a subject might have an eye disease comprising at least a portion of an LPH gene.

In some embodiments, the present invention provides computer implemented methods of determining a subject's (e.g. patient's) risk of developing eye disease comprising: a) providing: i) nucleic acid from a subject; wherein the nucleic acid comprises an LPH gene; and ii) a computer comprising software for the prediction of a subject's risk of developing eye disease; and b) detecting the presence of at least one variation in the subject's LPH gene to generate genetic variation information; c) inputting the genetic variation information into the computer; d) calculating the subject's risk with the software; and e) displaying the subject's risk (e.g. in a printout or on a display screen).

### DESCRIPTION OF THE FIGURES

FIG. 1 shows the cDNA nucleic acid sequence of human LPH3 (SEQ ID NO:1).

FIG. 2 shows the cDNA nucleic acid sequence of human LPH3 with additional 5′ and 3′ flanking regions (SEQ ID NO:2).

FIG. 3 shows a sequence alignment between the cDNA sequence of human LPH3 (SEQ ID NO:1), and accession Nos. AF307080 (human LEC3) and AB018311.

FIG. 4 shows both the nucleotide (SEQ ID NO:1) and peptide (SEQ ID NO:3) sequences of human LPH3.

FIG. 5 shows the amino acid sequence of human LPH3 (SEQ ID NO:3) and the amino acid sequence of human LEC3 (SEQ ID NO:4, accession no. AF307080).

FIG. 6 shows the exon regions of human genomic LPH3 shown in SEQ ID NO:5.

FIG. 7 shows the human genomic sequence of human LPH3 (SEQ ID NO:5), with the intronic/non-coding regions in lower case, and the exon regions shown in uppercase and bold.

FIG. 8 shows the exon regions of human genomic LPH1 shown in SEQ ID NO:32.

FIG. 9 shows the human genomic sequence of human LPH1 (SEQ ID NO:32), with the intronic/non-coding regions in lower case, and the exon regions shown in uppercase and bold.

FIG. 10 shows a hypothetical model of LPH-TIGR interaction. Importantly, the present invention is not limited to a particular mechanism of action. Indeed, an understanding of the mechanism of action is not necessary to practice the present invention. Nevertheless, this figure depicts a hypothetical model of LPH-TIGR interaction.

FIG. 11 shows the cDNA sequence of human TIGR/myocilin (SEQ ID NO:172, accession no. NM_{—}000261), and the amino acid sequence of human TIGR/myocilin (SEQ ID NO:173, accession no. NP-000252. Nucleotides 758-1528 of SEQ ID NO:172 encode an olfactomedin domain, and amino acids 246-502 of SEQ ID NO:173 represent an olfactomedin domain of the human TIGR peptide.

Definitions

To facilitate an understanding of the invention, a number of terms are defined below.

As used herein, the terms “subject” and “patient” refer to any animal, such as a mammal like a dog, cat, bird, livestock, and preferably a human. Specific examples of “subjects” and “patients” include, but are not limited to, individuals with glaucoma, individuals with glaucoma-related characteristics such as ocular hypertension, relatives of such individuals, and individuals at risk of glaucoma (e.g. individuals with a family history of glaucoma, and individuals over the age of 40).

As used herein, the term “eye disease” refers to diseases affecting all, or a portion of the eye, including, but not limited to, Primary Open-Angle Glaucoma (e.g. juvenile onset and adult onset), occular hypertension, and other eye diseases where, for example, the intraocular pressure is elevated.

The term “LPH gene” refers to a full-length LPH nucleotide sequence (e.g., SEQ ID NO:5 for LPH3, and SEQ ID NO:32 for LPH1). However, it is also intended that the term encompass fragments of the genomic LPH sequences, the cDNA sequences (e.g. SEQ ID NO:1) as well as other domains within the full-length LPH nucleotide sequences. Furthermore, the terms “LPH nucleotide sequence” or “LPH polynucleotide sequence” encompasses DNA, cDNA, and RNA (e.g., MRNA) sequences.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the term “variant” refers to a gene or gene product that displays modifications in sequence and/or functional properties (i.e., altered characteristics) when compared to the wild-type gene or gene product. Examples of variants include, but are not limted to, polymorphisms and mutations. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “the complement of” a given sequence is used in reference to the sequence that is completely complementary to the sequence over its entire length. For example, the sequence A-G-T-A is “the complement” of the sequence T-C-A-T.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on CDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency.

As used herein, the term “competes for binding” is used in reference to a first polypeptide with an activity which binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Kₘ” as used herein refers to the Michaelis-Menton constant for an enzyme and is defined as the concentration of the specific substrate at which a given enzyme yields one-half its maximum velocity in an enzyme catalyzed reaction.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ, and in some cases the Tₘ may be determined empirically by beginning with the calculated Tₘ and testing small increases or decreases of temperature and examining the effect on the population of nucleic acid molecules.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5X SSPE (43.8 g/l NaCl, 6.9 g/l NaH₂PO₄ H₂0 and 1.85 g/l EDTA, pH adjusted to 7.4 with NaOH), 0.5% SDS, 5X Denhardt's reagent and 100 μg/ml denatured salmon sperm DNA followed by washing in a solution comprising 0.1X SSPE, 1.0% SDS at 42 C when a probe of about 500 nucleotides in length is employed.

“Medium stringency conditions” when used in reference to nucleic acid hybridization comprise conditions equivalent to binding or hybridization at 42° C. in a solution consisting of 5X SSPE (43.8 g/l NaCl, 6.9 g/l NaH₂PO₄ H₂0 and 1.85 g/l EDTA, pH adjusted to 7.4 with NaOH), 0.5% SDS, 5X Denhardt's reagent and 100 μg/ml denatured salmon sperm DNA followed by washing in a solution comprising 1.0X SSPE, 1.0% SDS at 42 C when a probe of about 500 nucleotides in length is employed.

“Low stringency conditions” comprise conditions equivalent to binding or hybridization at 42 C in a solution consisting of 5X SSPE (43.8 g/l NaCl, 6.9 g/l NaH₂PO₄ H₂O and 1.85 g/l EDTA, pH adjusted to 7.4 with NaOH), 0. 1% SDS, 5X Denhardt's reagent [50X Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5X SSPE, 0.1% SDS at 42 C when a probe of about 500 nucleotides in length is employed.

The following terms are used to describe the sequence relationships between two or more polynucleotides: “reference sequence”, “sequence identity”, and “percentage of sequence identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length (e.g. SEQ ID NO:1 may be used as a reference sequence). Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The window of comparison, as used in the present application, is the entire length of the recited reference sequence (i.e. if SEQ ID NO:1 is recited as the reference sequence, percentage of sequence identity is compared over the entire length of SEQ ID NO:1).

The term “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides (preferably 25-100 nucleotides), wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention. As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine (e.g., change the valine at position 73 in SEQ ID NO:3 to leucine by changing the G at position 217 of SEQ ID NO:1 to a C); a group of amino acids having aliphatic-hydroxyl side chains is serine and threonine (e.g., change the serine at position 23 in SEQ ID NO:3 to threonine by changing the G at position 68 of SEQ ID NO:1 to a C); a group of amino acids having amide-containing side chains is asparagine and glutamine (e.g., change the glutamine at position 37 of SEQ ID NO:3 to an asparagine by changing the G at position 111 of SEQ ID NO:1 to a C); a group of amino acids having aromatic side chains is phenylalanine, tyrosine, and tryptophan (e.g., change the tyrosine at position 107 in SEQ ID NO:3 to tryptophan by changing the AT at positions 320-21 of SEQ ID NO:1 to GG); a group of amino acids having basic side chains is lysine, arginine, and histidine (e.g., change the histidine at position 35 in SEQ ID NO:3 to arginine by changing the A at position 104 of SEQ ID NO:1 to a G); and a group of amino acids having sulfur-containing side chains is cysteine and methionine (e.g., change the cystein at position 104 in SEQ ID NO:3 to methionine by changing TGT at positions 310-12 of SEQ ID NO:1 to ATG). Preferred conservative amino acids substitution groups are: valine-leucine-isoleucine, phenylalanine-tyrosine, lysine-arginine, alanine-valine, and asparagine-glutamine.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions with its various ligands and/or substrates.

As used herein, the term “genetic variation information” or “genetic variant information” refers to the presence or absence of one or more variant nucleic acid sequences (e.g., polymorphism or mutations) in a given allele of a particular gene (e.g., the human LPH3 gene).

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (D. L. Kacian et al., Proc. Natl. Acad. Sci. USA 69:3038 [1972]). Other nucleic acids will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention may be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to a nucleic acid sequence or structure to be detected or characterized. Thus, the “target” is sought to be sorted out from other nucleic acid sequences. A “segment” is defined as a region of nucleic acid within the target sequence.

As used herein, the term “polymerase chain reaction” (“PCR”) refers to the method described by Kleppe, et al., J. Molecular Biology, 56, 1971, pp. 341-361, and in U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “antisense” is used in reference to RNA sequences that are complementary to a specific RNA sequence (e.g., MRNA). Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding LPH3 includes, by way of example, such nucleic acid in cells ordinarily expressing LPH3 (e.g. trabecular meshwork cells) where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i. e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a MRNA molecule. The coding region is bounded, in eukaryotes, on the 5′ side by the nucleotide triplet “ATG” that encodes the initiator methionine and on the 3′ side by one of the three triplets, which specify stop codons (i.e., TAA, TAG, TGA).

As used herein, the term “purified” or “to purify” refers to the removal of contaminants from a sample. For example, LPH3 antibodies are purified by removal of contaminating non-immunoglobulin proteins; they are also purified by the removal of immunoglobulin that does not bind LPH3. The removal of non-immunoglobulin proteins and/or the removal of immunoglobulins that do not bind LPH3 results in an increase in the percent of LPH3-reactive immunoglobulins in the sample. In another example, recombinant LPH3 polypeptides are expressed in bacterial host cells and the polypeptides are purified by the removal of host cell proteins; the percent of recombinant LPH3 polypeptides is thereby increased in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al, supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “transgene” as used herein refers to a foreign, heterologous, or autologous gene that is placed into an organism by introducing the gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene. The term “autologous gene” is intended to encompass variants (e.g., polymorphisms or mutants) of the naturally occurring gene. The term transgene thus encompasses the replacement of the naturally occurring gene with a variant form of the gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots). The amount of MRNA present in the band corresponding in size to the correctly spliced LPH (e.g. human LPH3) transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

A “composition comprising a given polynucleotide sequence” as used herein refers broadly to any composition containing the given polynucleotide sequence. The composition may comprise an aqueous solution. Compositions comprising polynucleotide sequences encoding LPH3 (e.g., SEQ ID NO:1) or fragments thereof may be employed as hybridization probes. In this case, the LPH3 encoding polynucleotide sequences are typically employed in an aqueous solution containing salts (e.g., NaCl), detergents (e.g., SDS), and other components (e.g., Denhardt's solution, dry milk, salmon sperm DNA, etc.).

The term “test compound” or “candidate compound” refer to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “response,” when used in reference to an assay, refers to the generation of a detectable signal (e.g., accumulation of reporter protein, increase in ion concentration, accumulation of a detectable chemical product).

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat Nos., 6,074,859, incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the phrase “computer readable medium encodes a representation” of a nucleic acid or amino acid sequence, refers to computer readable medium that has stored thereon information, that when delivered to a processor, allows the sequence of the nucleic or amino acid sequence to be displayed to a user (e.g. printed out or presented on a display screen).

As used herein, the terms “processor” and “central processing unit” or “CPU” are used interchangeably and refer to a device that is able to read a program from a computer memory (e.g., ROM or other computer memory) and perform a set of steps according to the program.

### DESCRIPTION OF THE INVENTION

The present invention relates to Latrophilin (LPH) polynucleotides and polypeptides, and to identifying and treating subjects at risk for eye disease. Specifically, the present invention provides novel human LPH3 and LPH1 polynucleotides and LPH3 polypeptides, assays for detecting variations in LPH polynucleotides associated with eye disease, such as glaucoma and related diseases, and methods and compositions for treating eye disease. The description of the invention provided below is presented in four sections: 1) human LPH3 and LPH1 polynucleotides and human LPH3 polypeptides; 2) compound screening using LPH; 3) detecting variations in LPH polynucleotides; and 4) LPH therapeutics.

I. Human LPH Polynucleotides and Polypeptides

The present invention provides novel human Latrophillin 3 (LPH3) and human Latrophillin 1 (LPH1) polynucleotides and polypeptides. These polypeptides and polynucleotides are described in more detail below.

A. LPH3 and LPH1 Polynucleotides

The present invention provides human LPH3 and LPH1 nucleic acid molecules. Thus, in some embodiments, the present invention provides an isolated and purified nucleic acid sequence comprising the cDNA sequence of human LPH3 as shown in SEQ ID NO:1, or fragments or variants thereof. In other embodiments, the present invention provides an isolated and purified nucleic acid sequence comprising the mRNA sequence of human LPH3 (e.g. replace the T's in SEQ ID NO:1 or 2 with U's), or fragements or variants thereof. In particular embodiments, the present invention provides an isolated and purified nucleic acid sequence comprising the cDNA sequence of human LPH3, further comprising 5′ and/or 3′ untranslated regions as shown in SEQ ID NO:2, or fragments or variants thereof. In some embodiments, the present invention provides isolated and purified nucleic acid sequences comprising the human gene sequence of LPH3 (shown in SEQ ID NO:5), or fragments or variants thereof. In other embodiments, the present invention provides isolated and purified nucleic acid sequences comprising the human gene sequence for LPH1 (shown in SEQ ID NO:32), or fragments or variants thereof.

In some embodiments, the nucleic acid sequences of the present invention encode an LPH3 peptide (e.g. SEQ ID NO:3). In other embodiments, the nucleic acid sequences comprises at least one exon encoding region selected from: SEQ ID NOS:6-30. In some embodiments, the nucleic acid sequence includes at least exon 1.1 (SEQ ID NO:7). In other embodiments, the nucleic acid sequence includes at least exon 1.1 (SEQ ID NO:7) and at least one other human LPH3 exon sequence (e.g. at least one selected from SEQ ID NOS:6 and 8-30). In some embodiments, the nucleic acid sequence comprises exon 3 (SEQ ID NO:10) and exon 4 (SEQ ID NO:11).

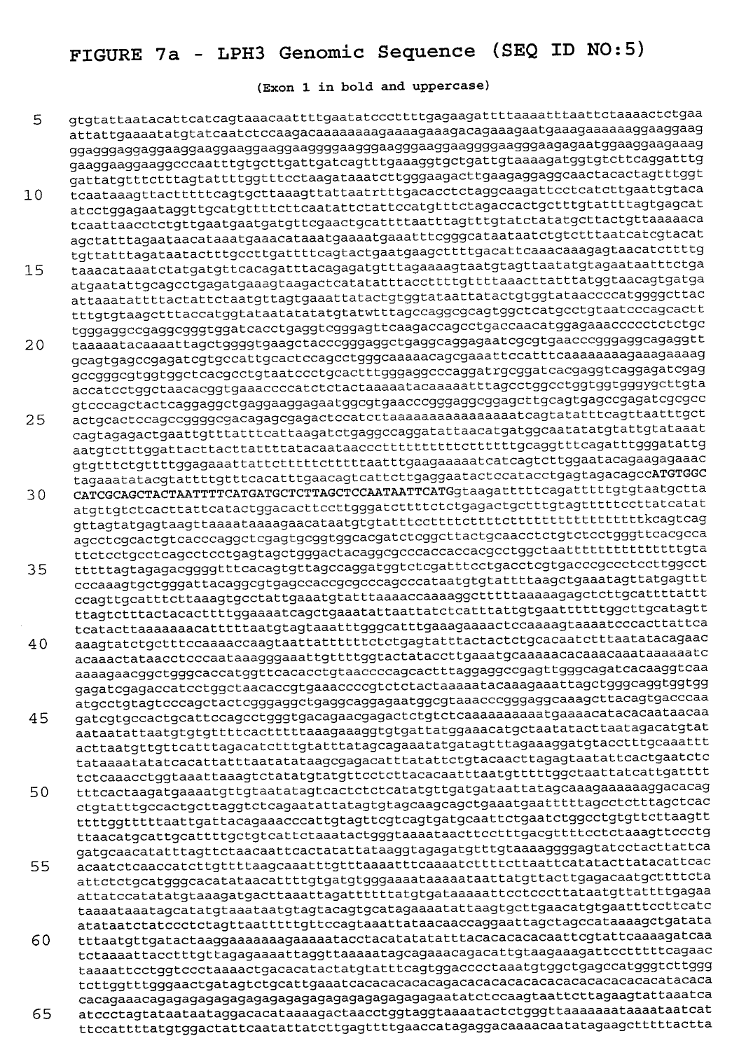

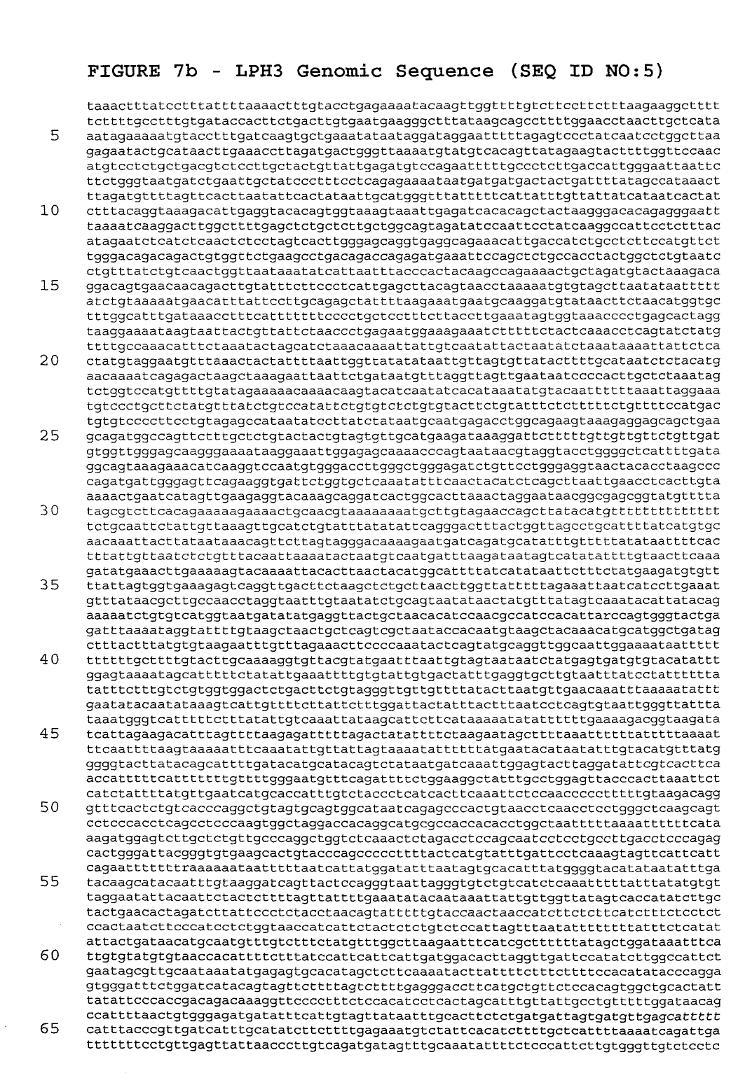

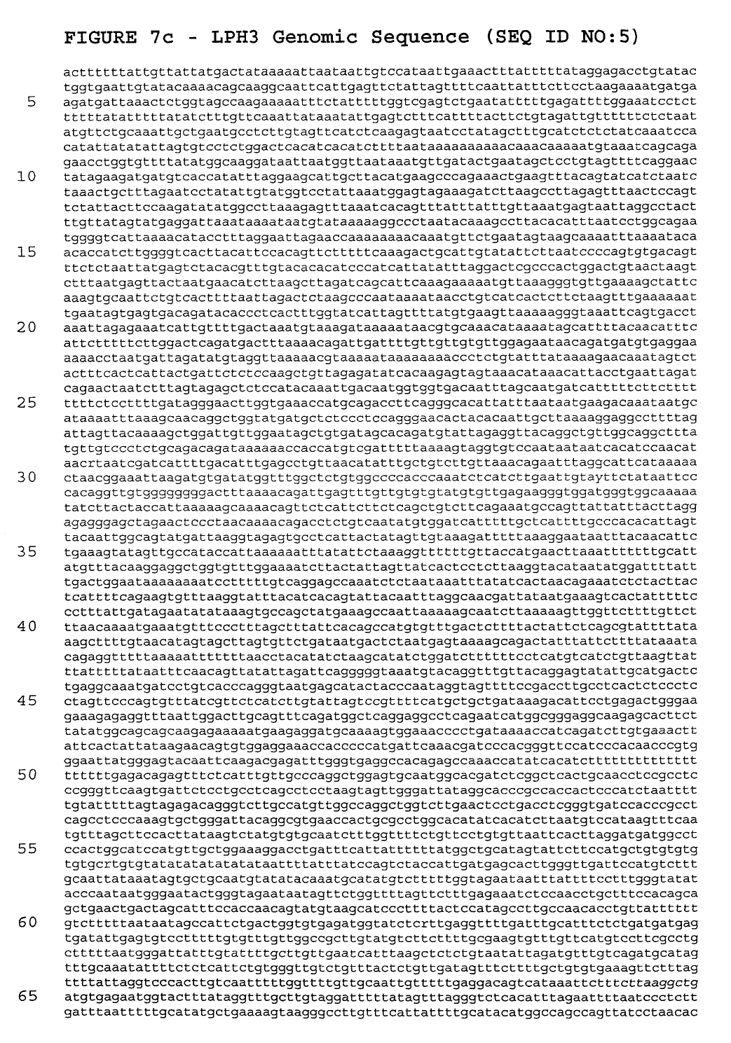

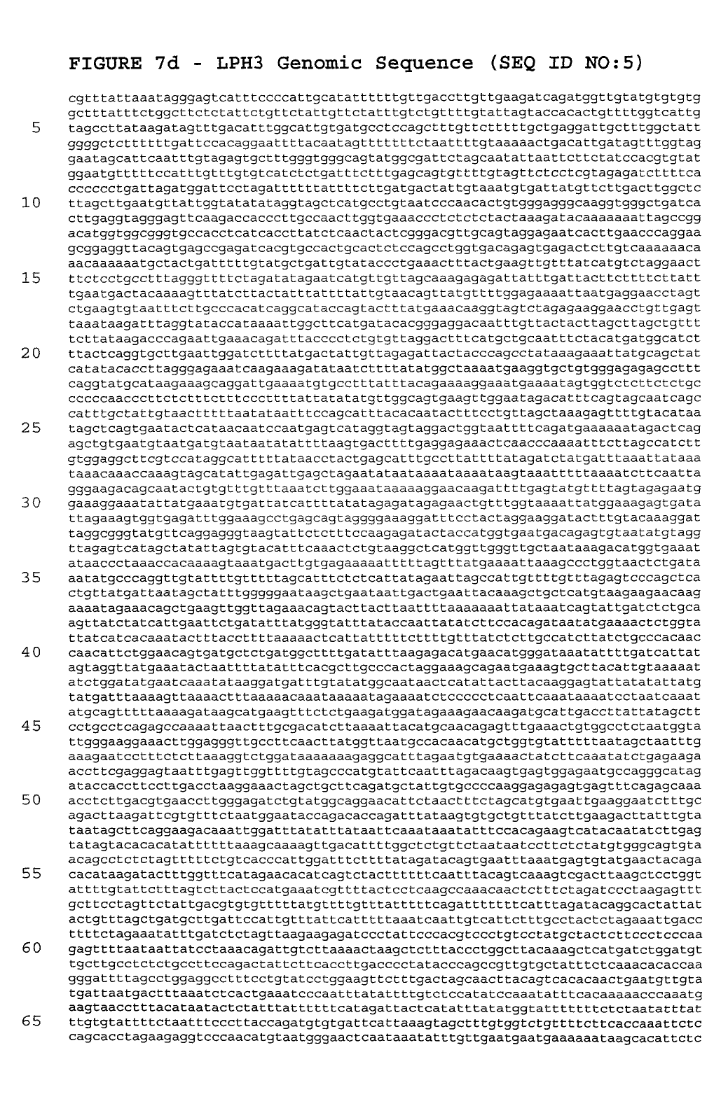

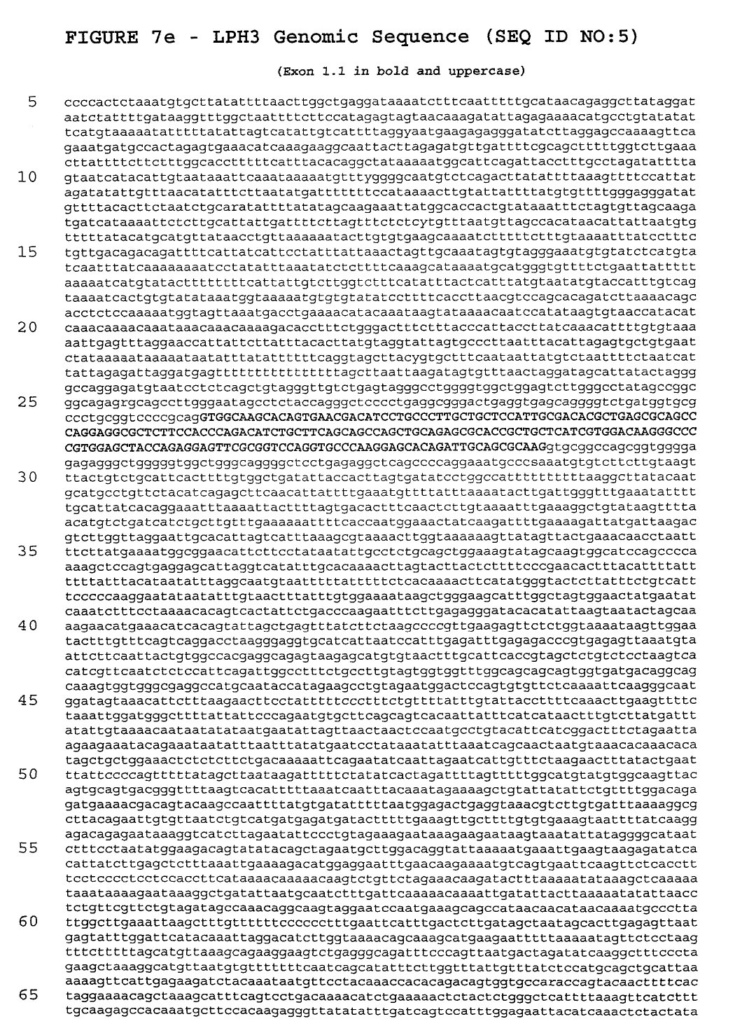

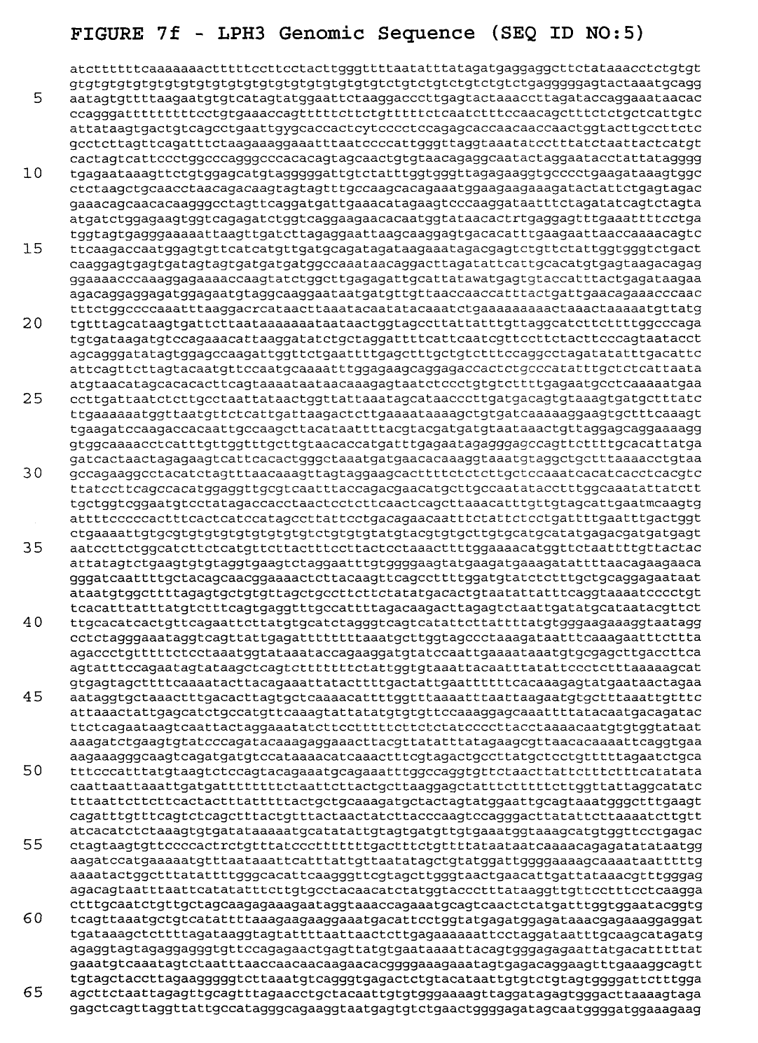

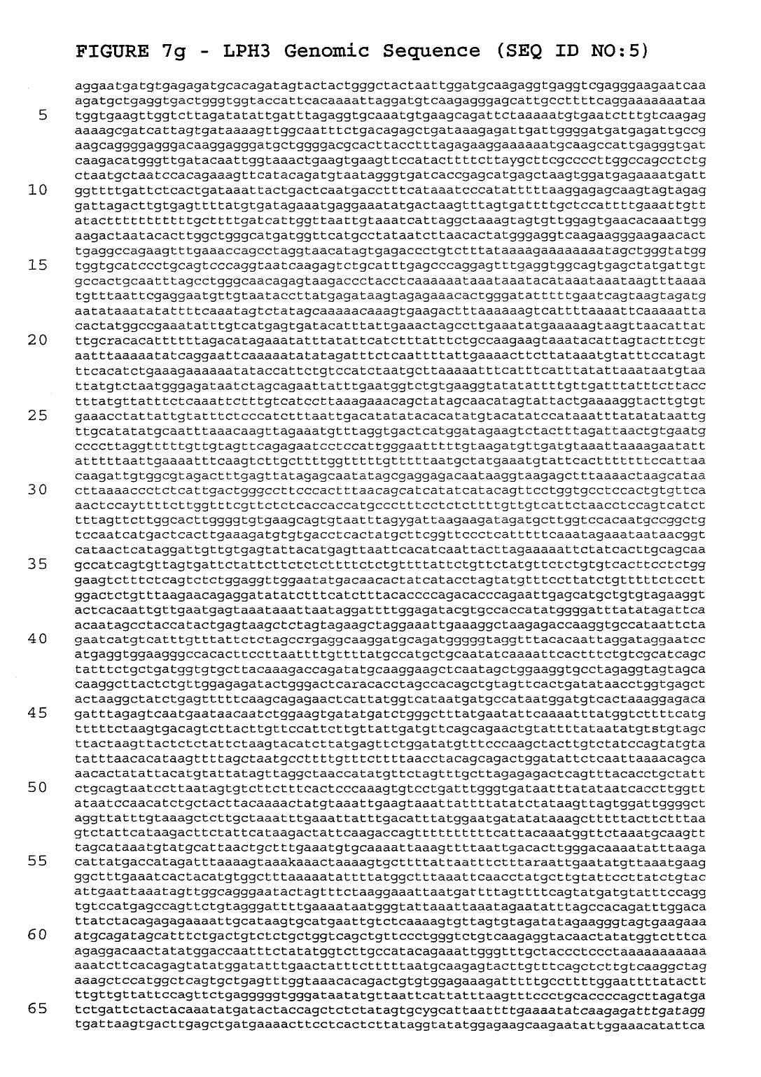

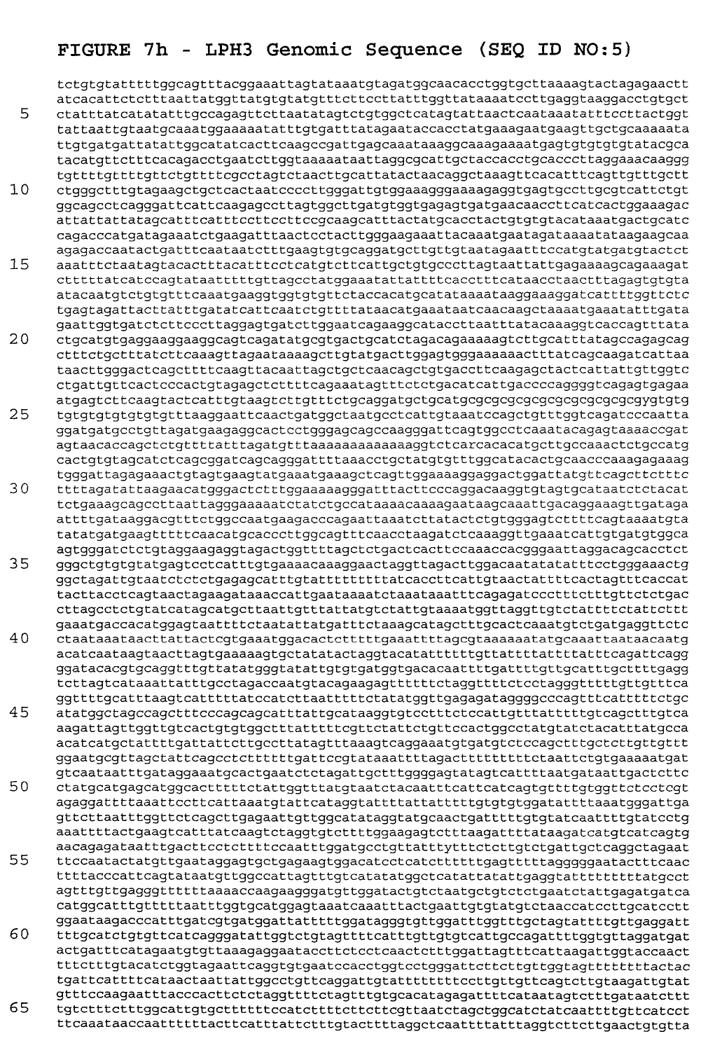

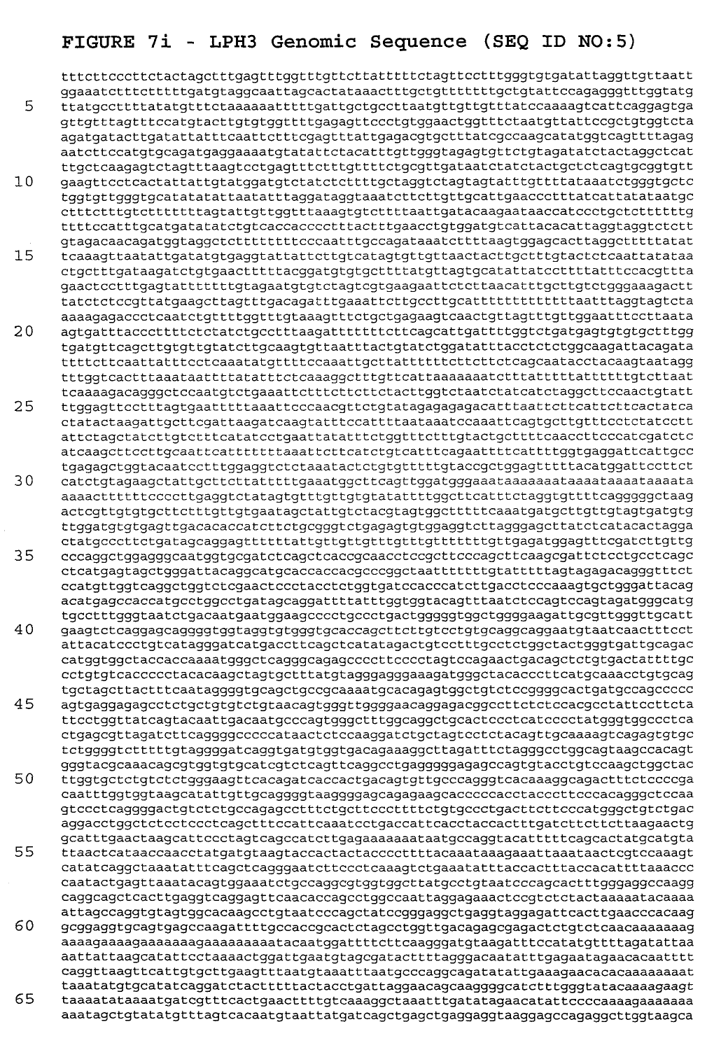

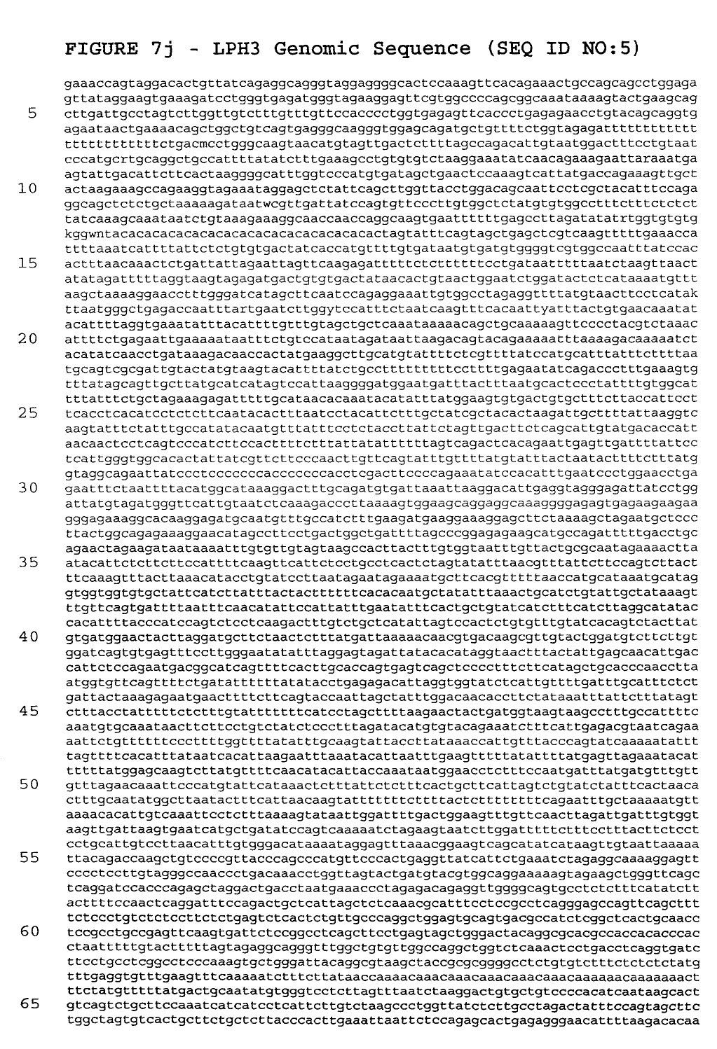

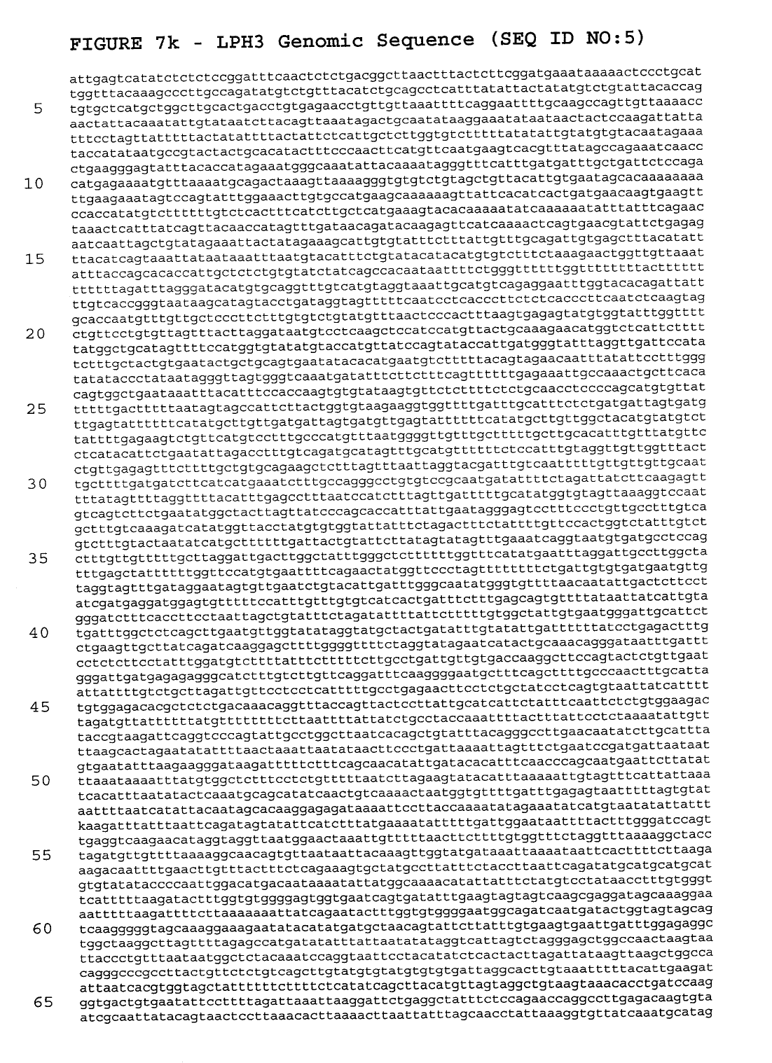

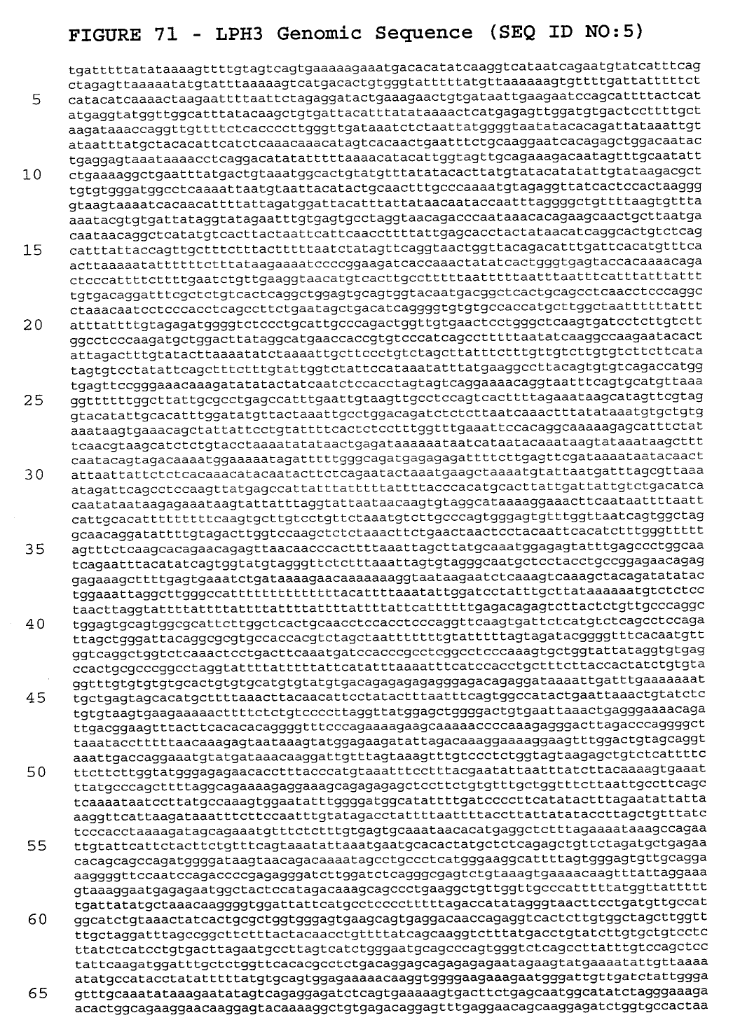

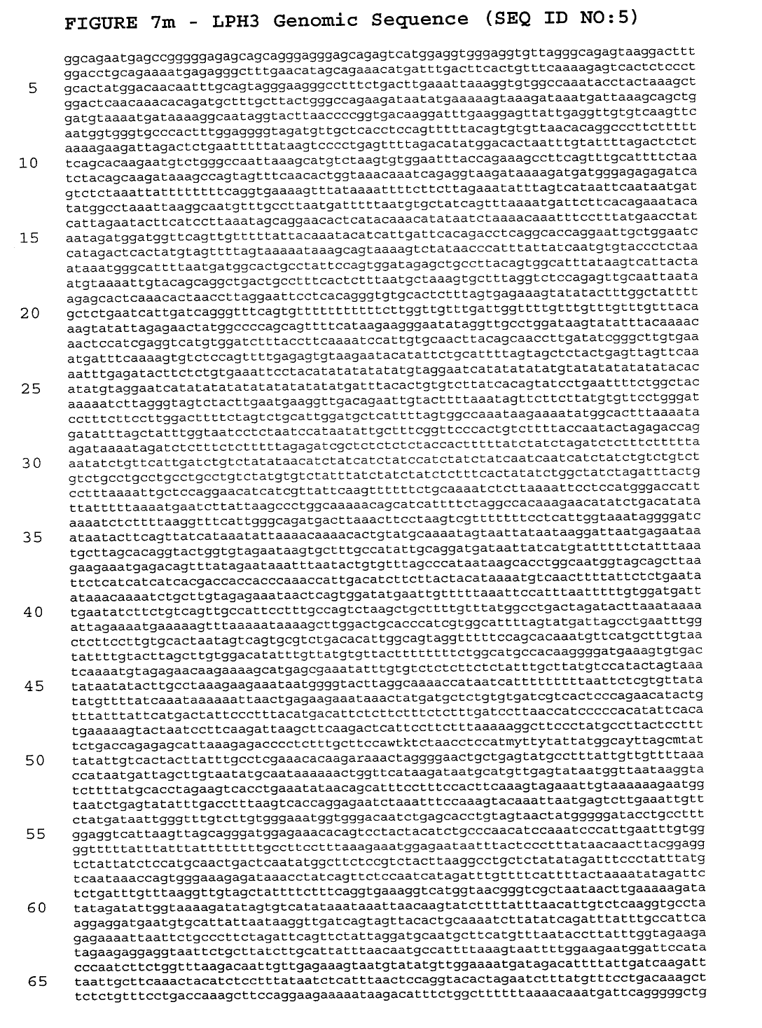

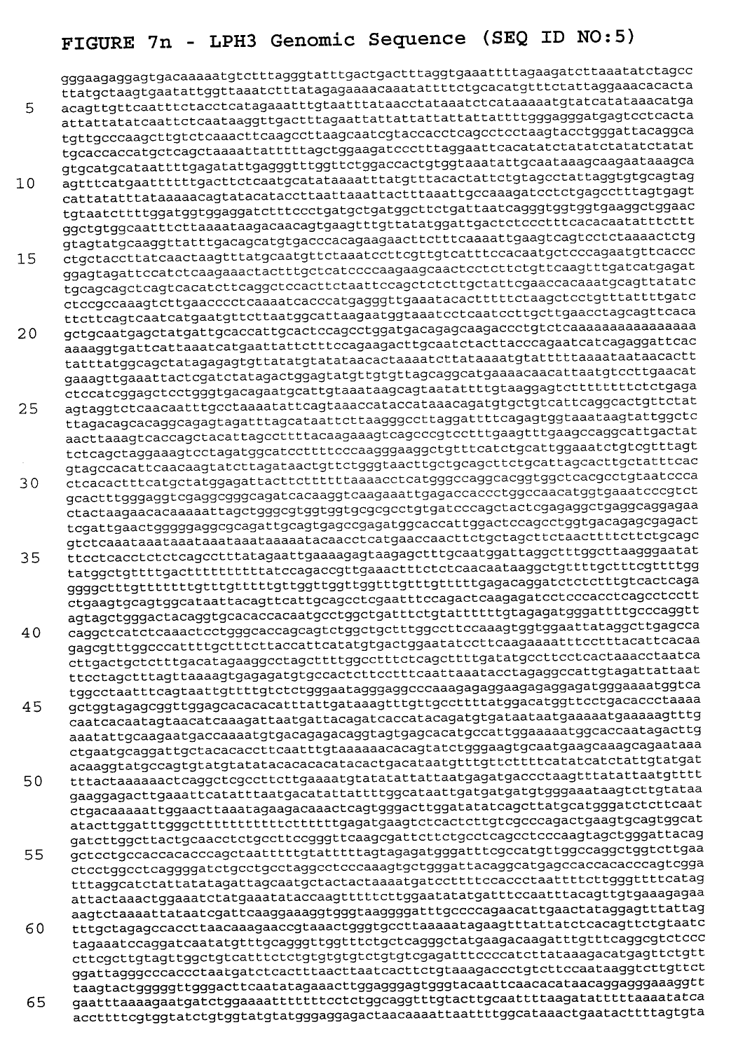

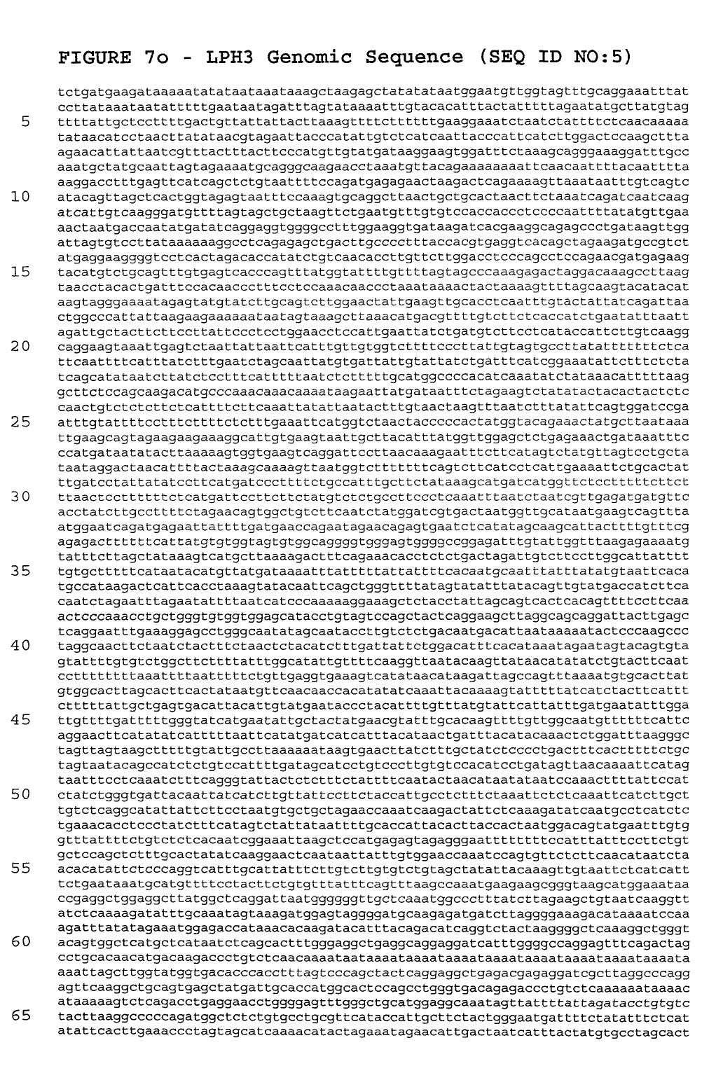

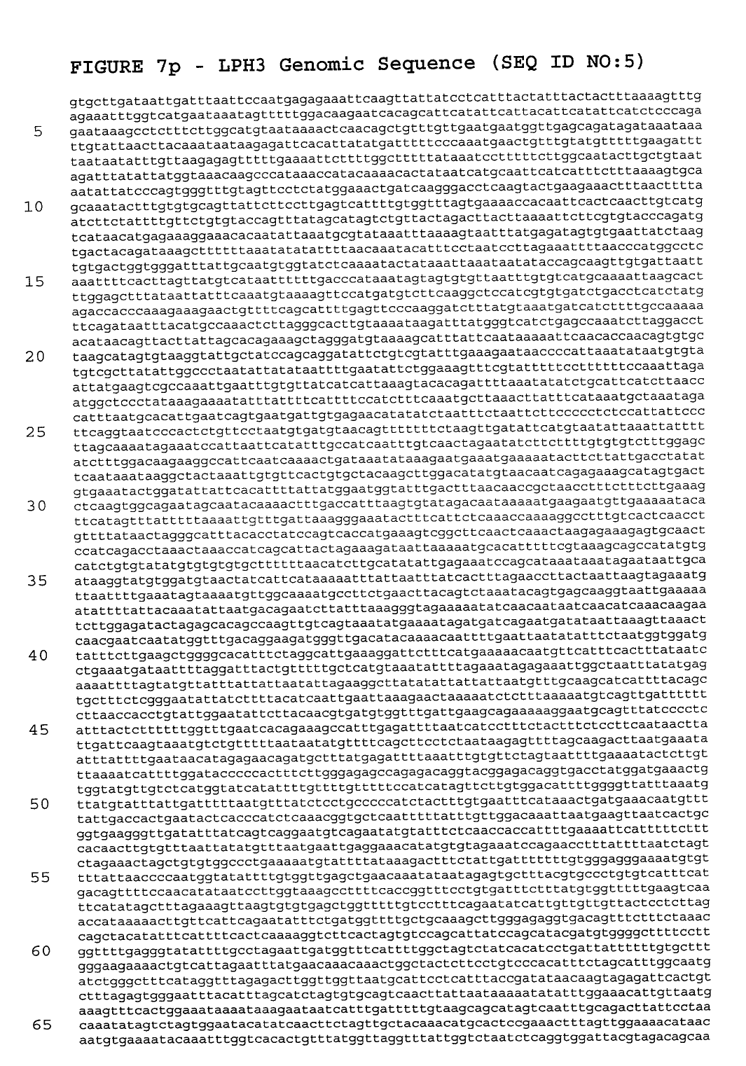

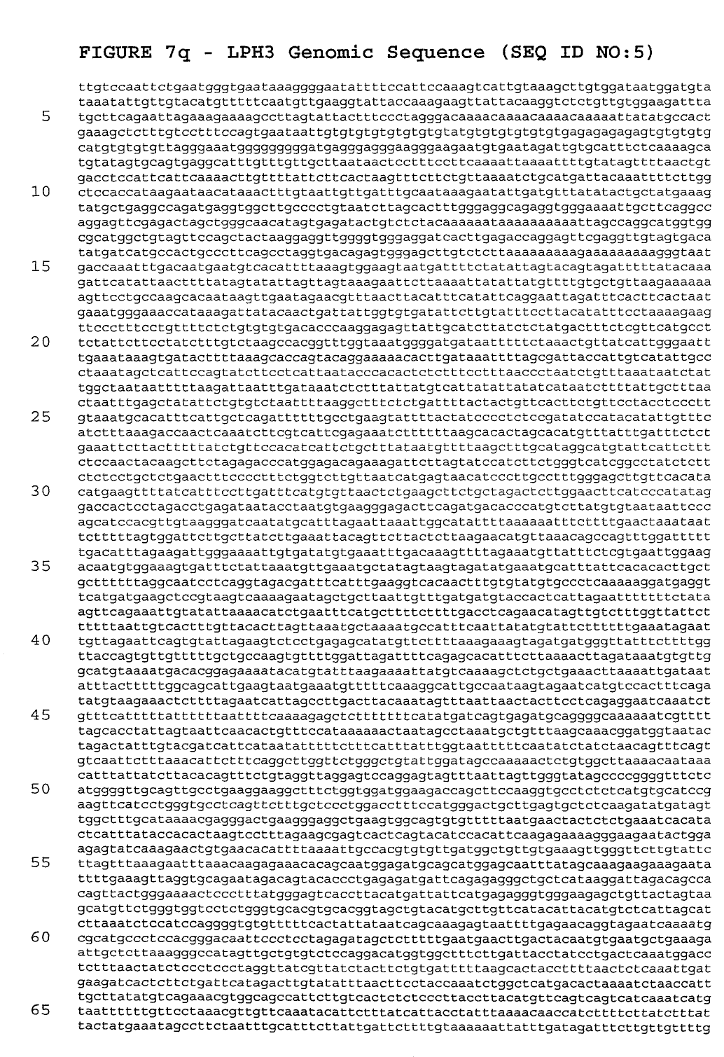

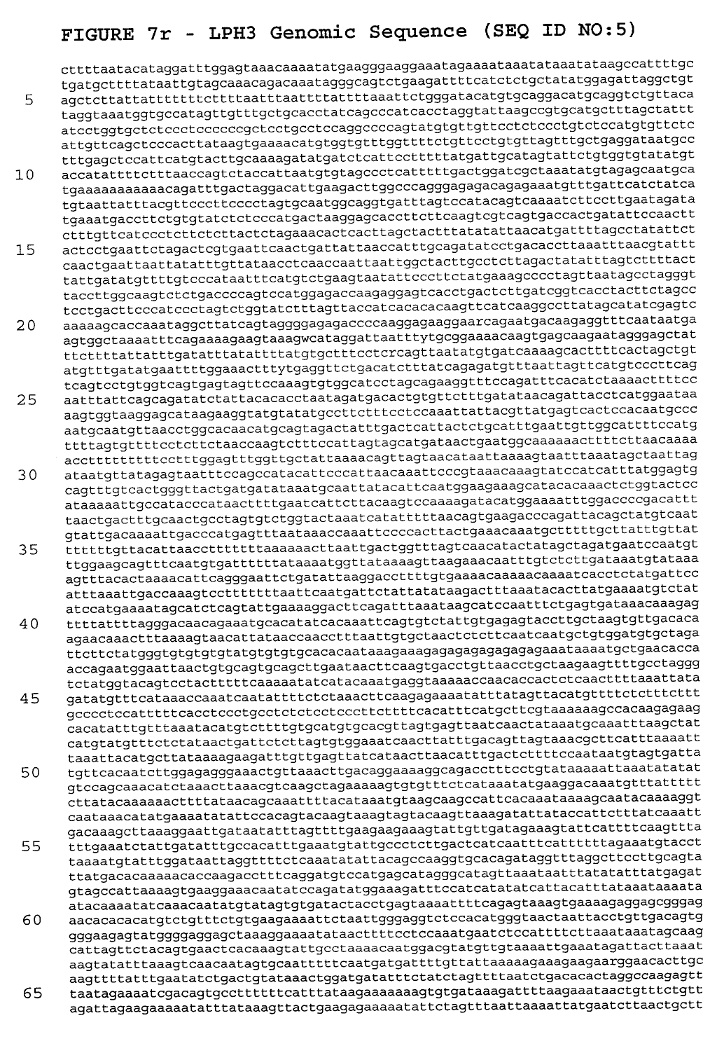

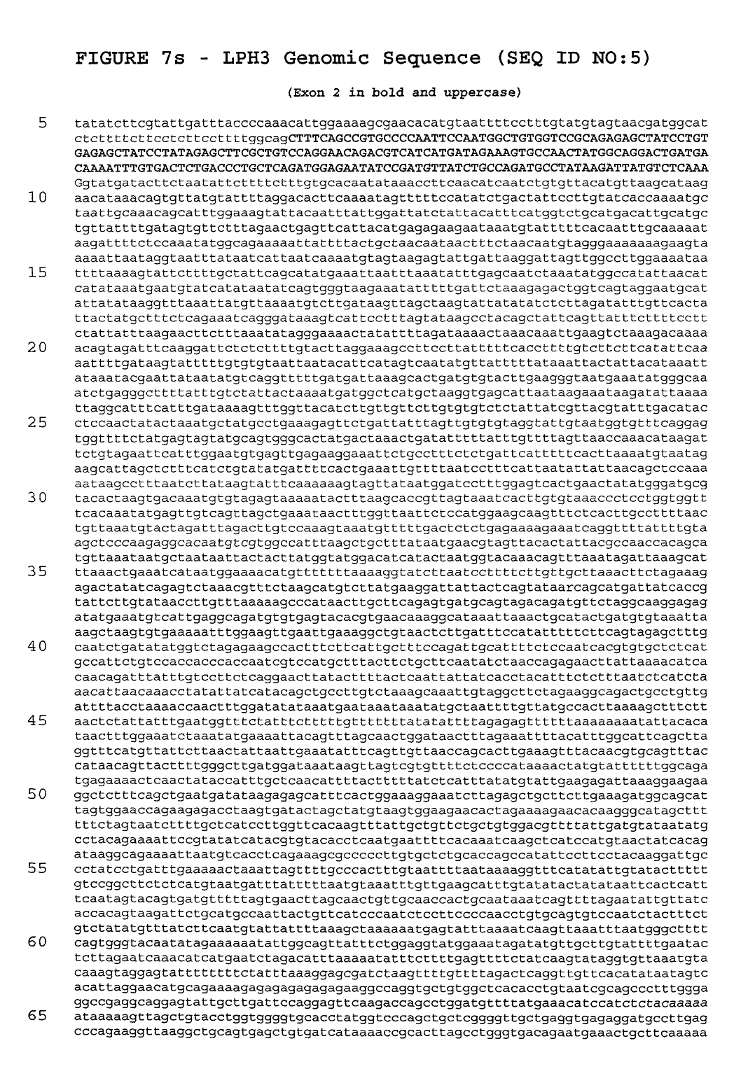

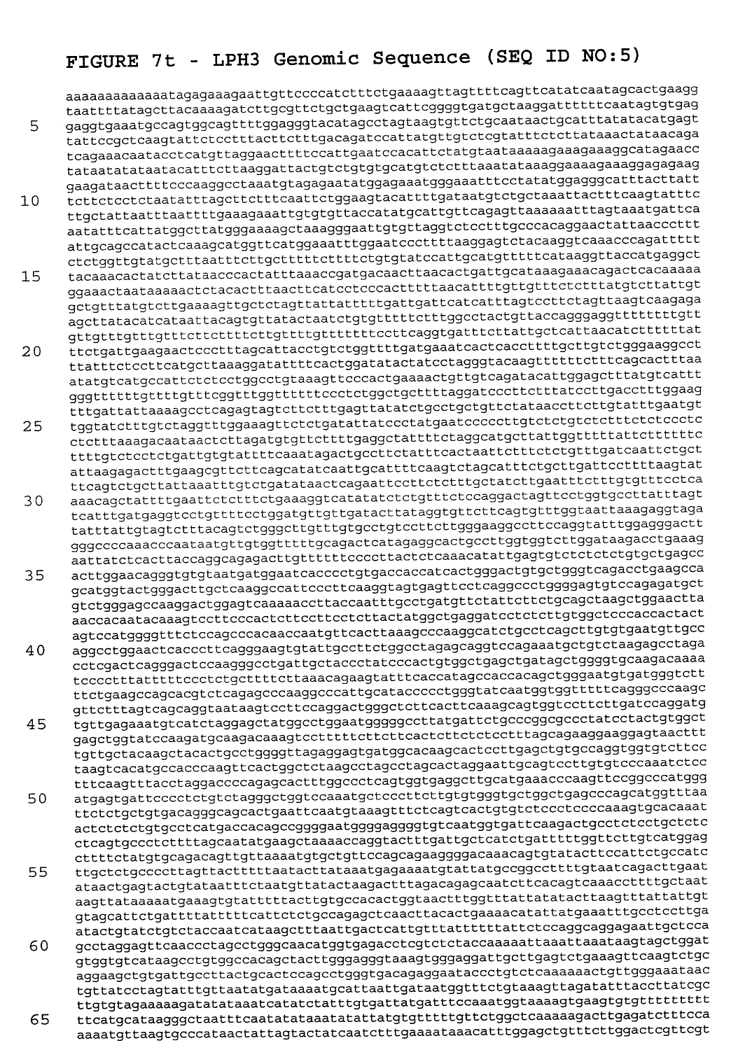

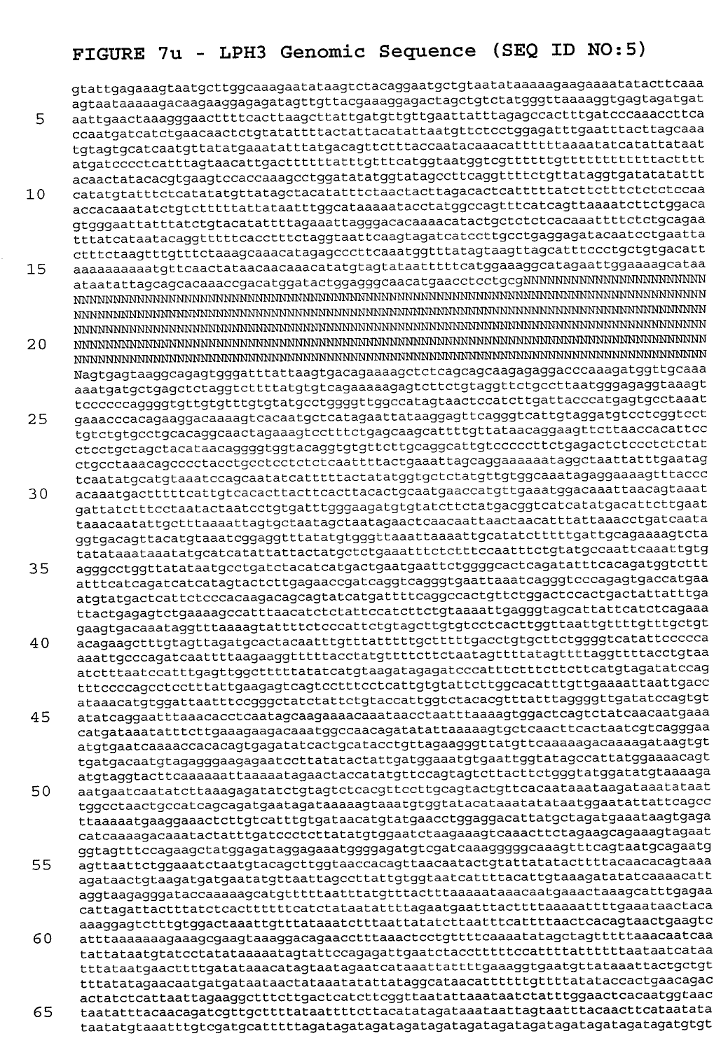

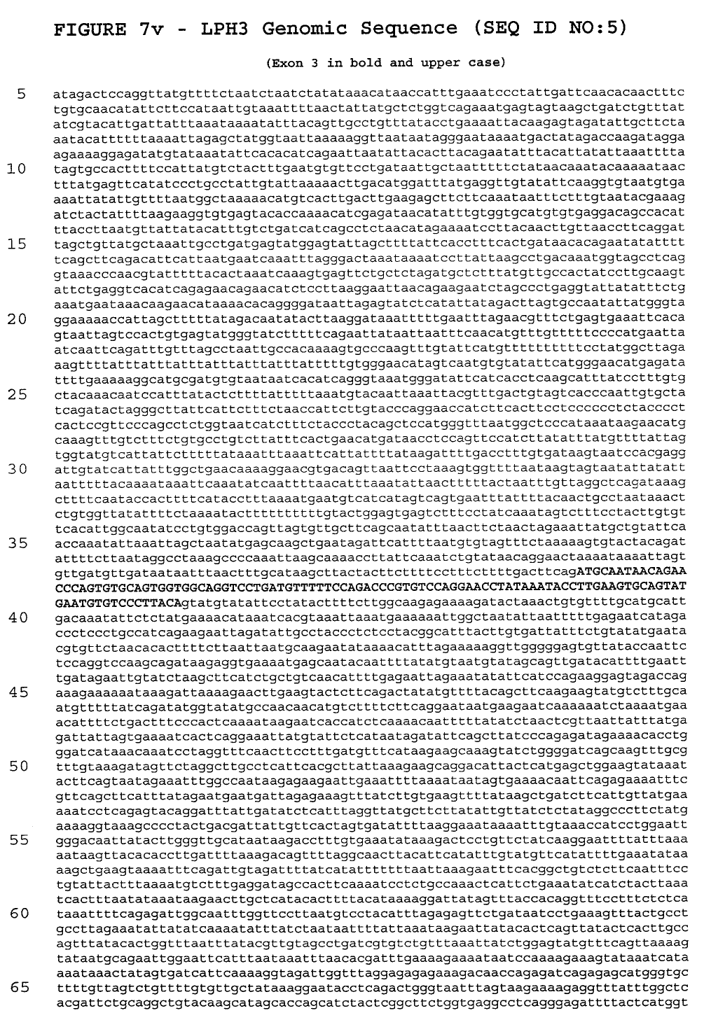

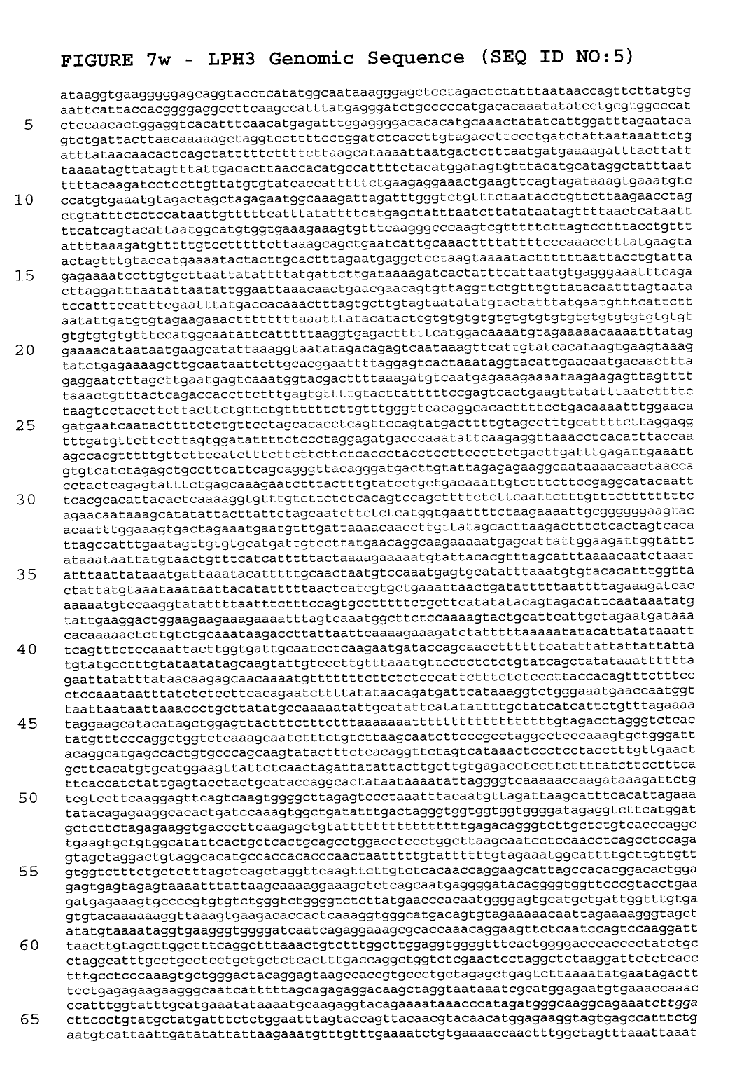

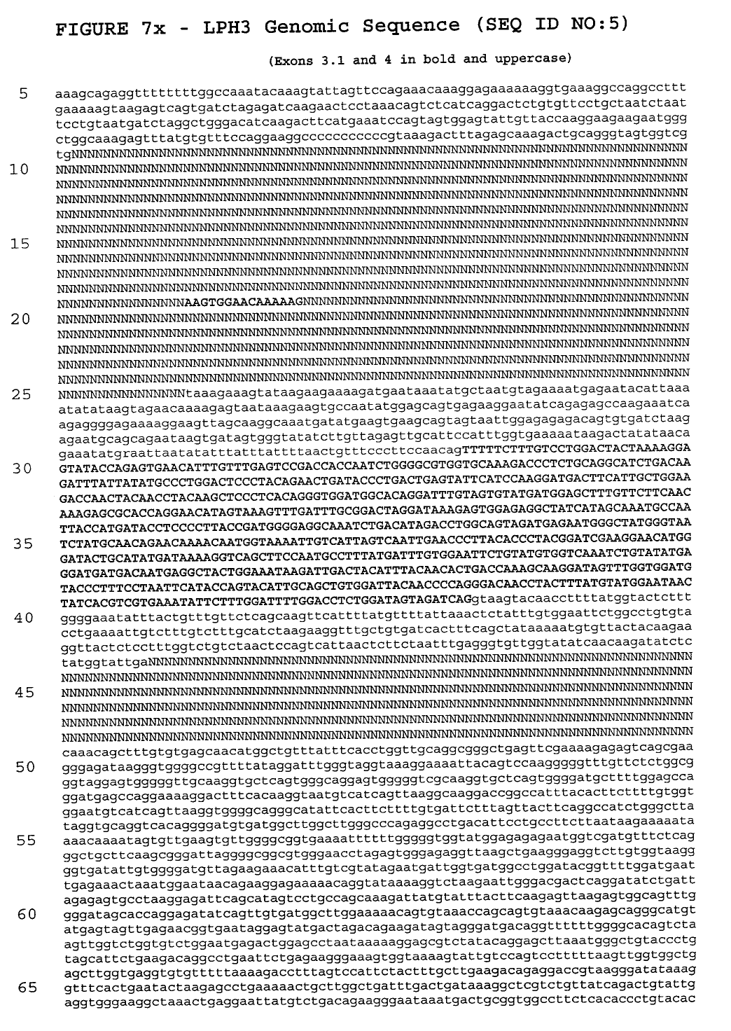

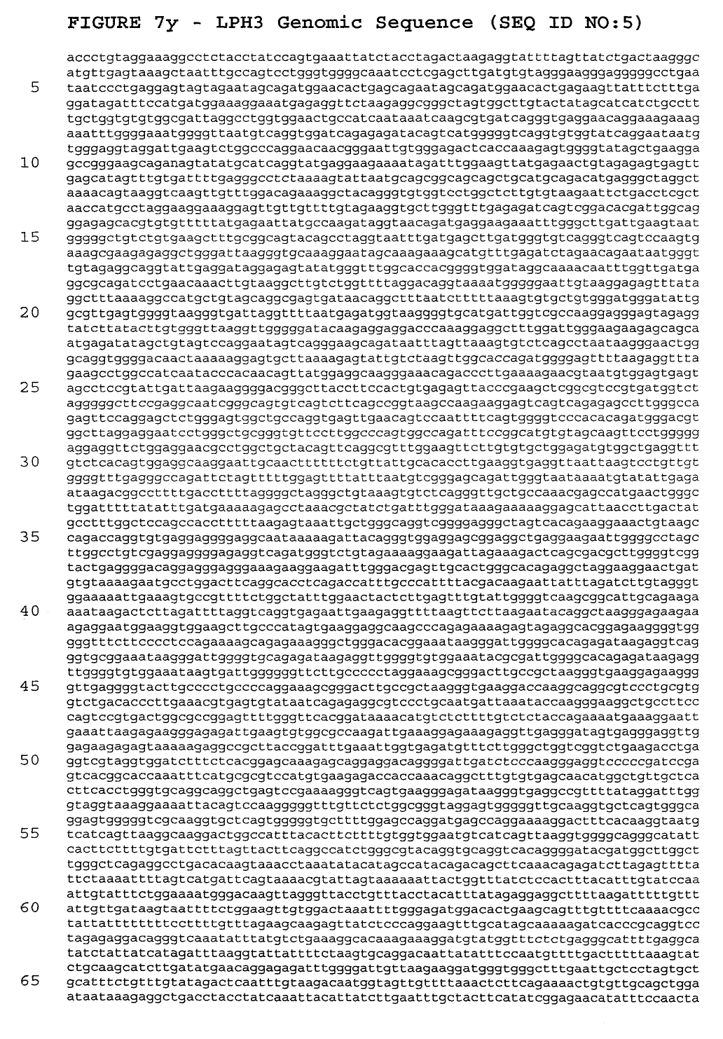

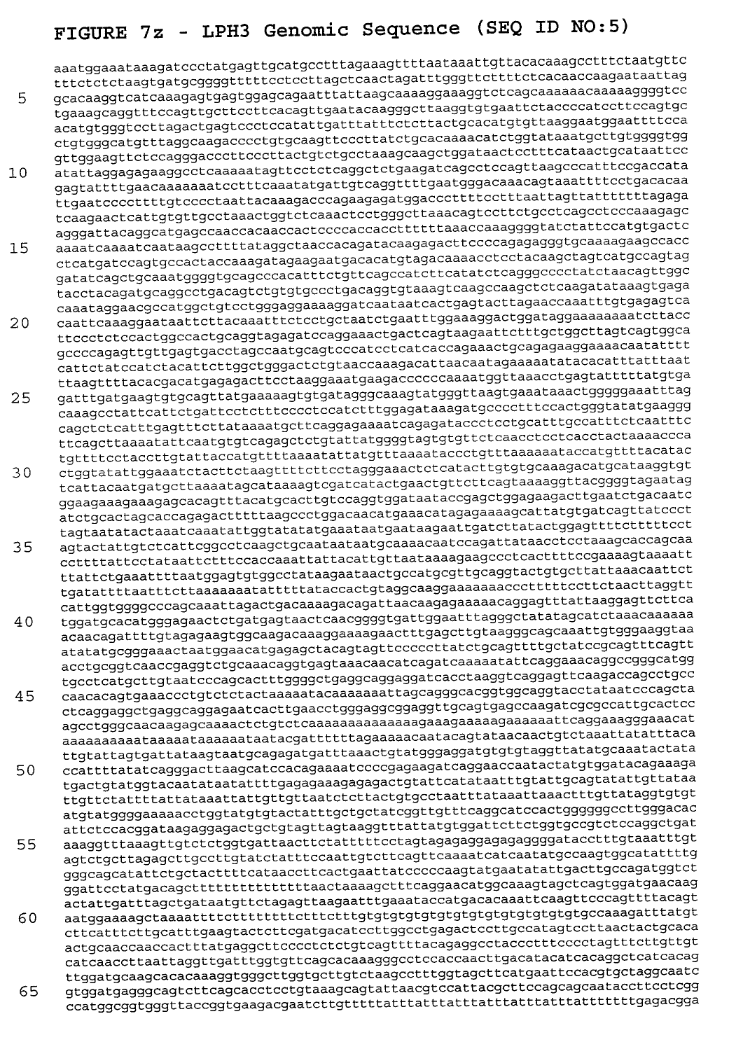

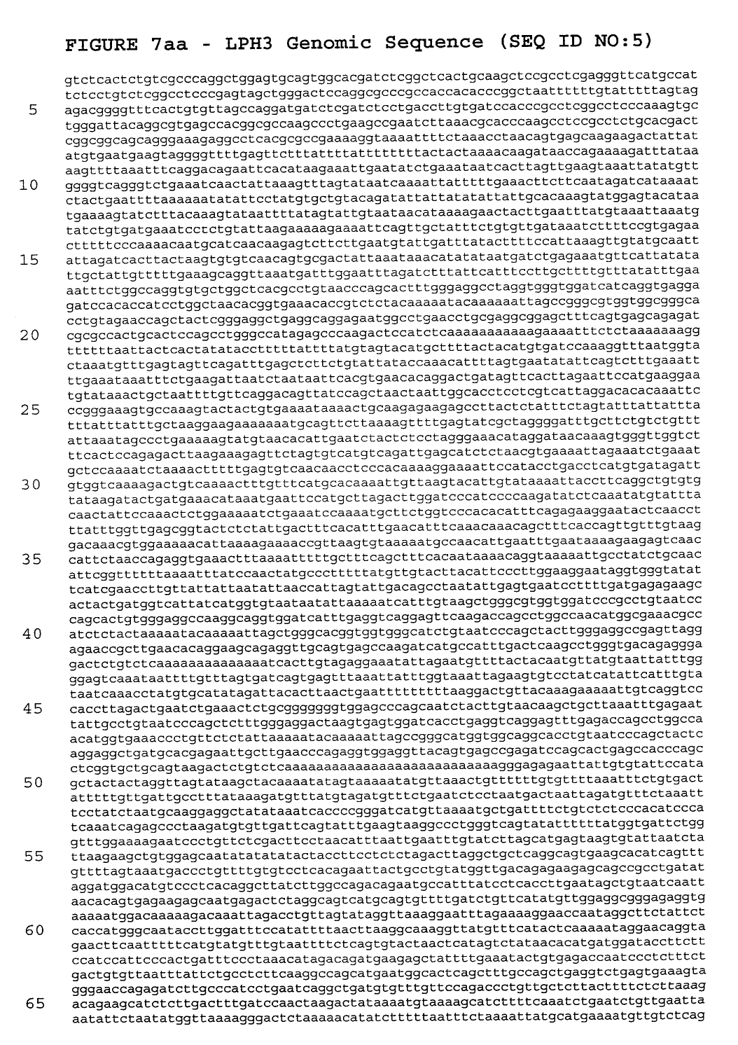

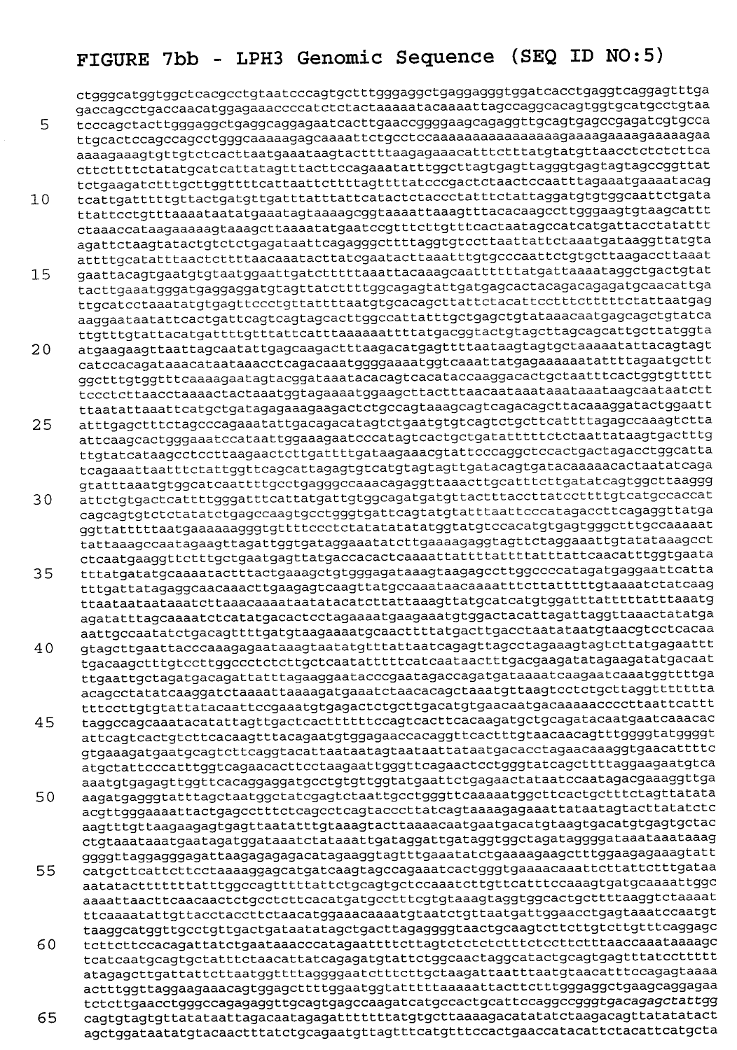

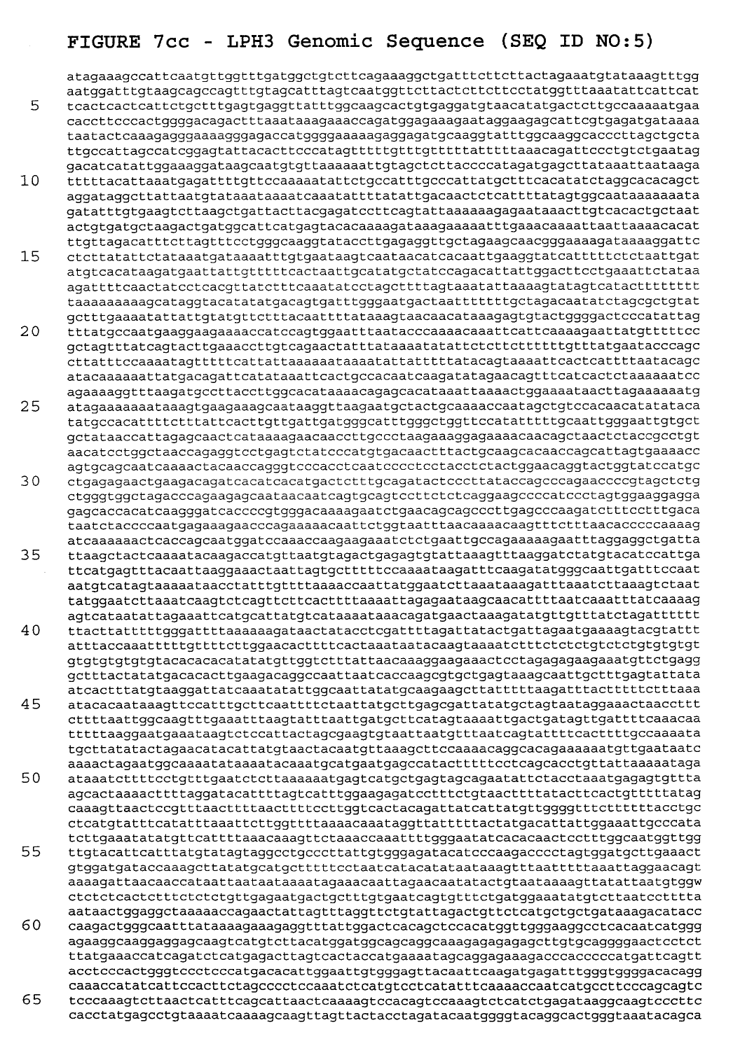

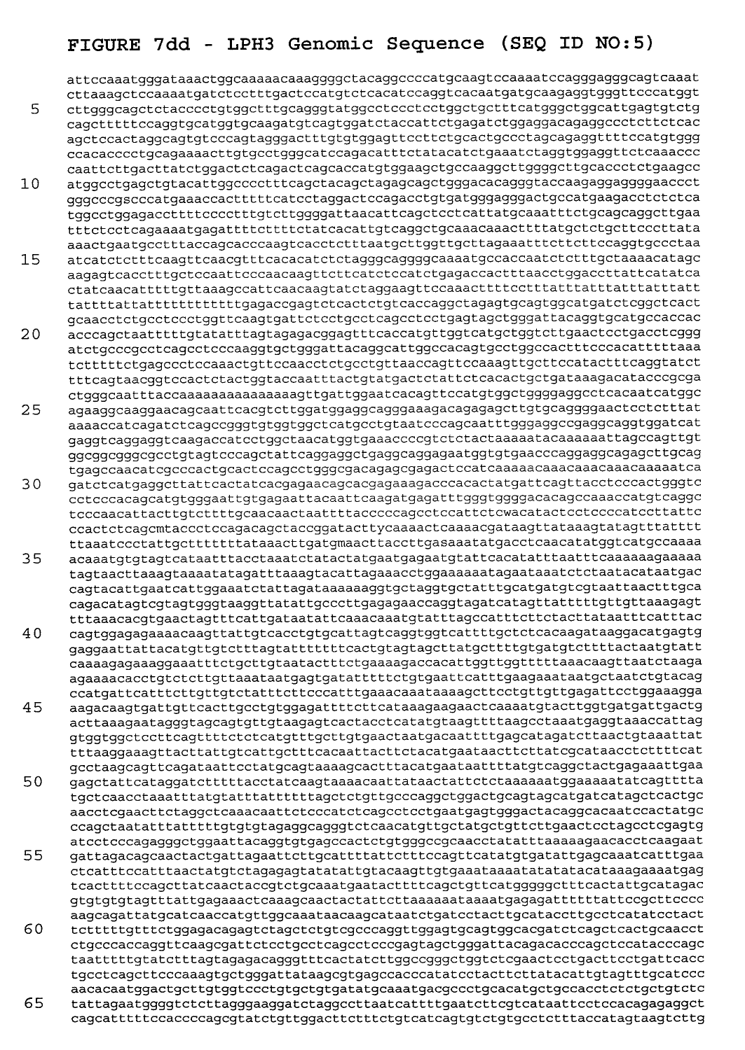

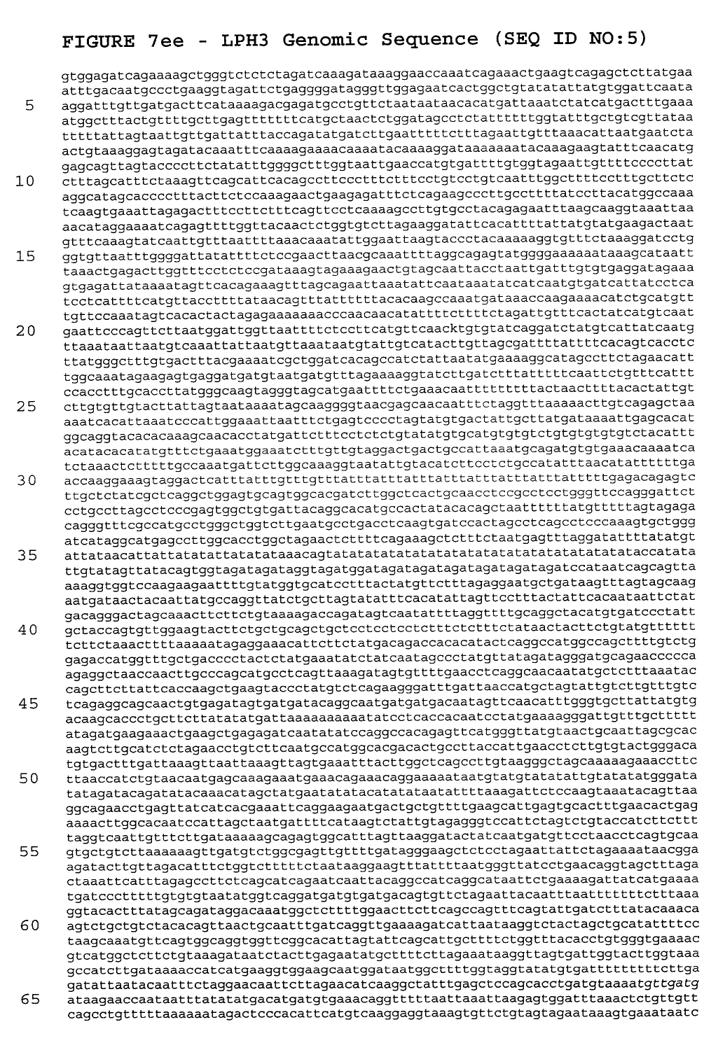

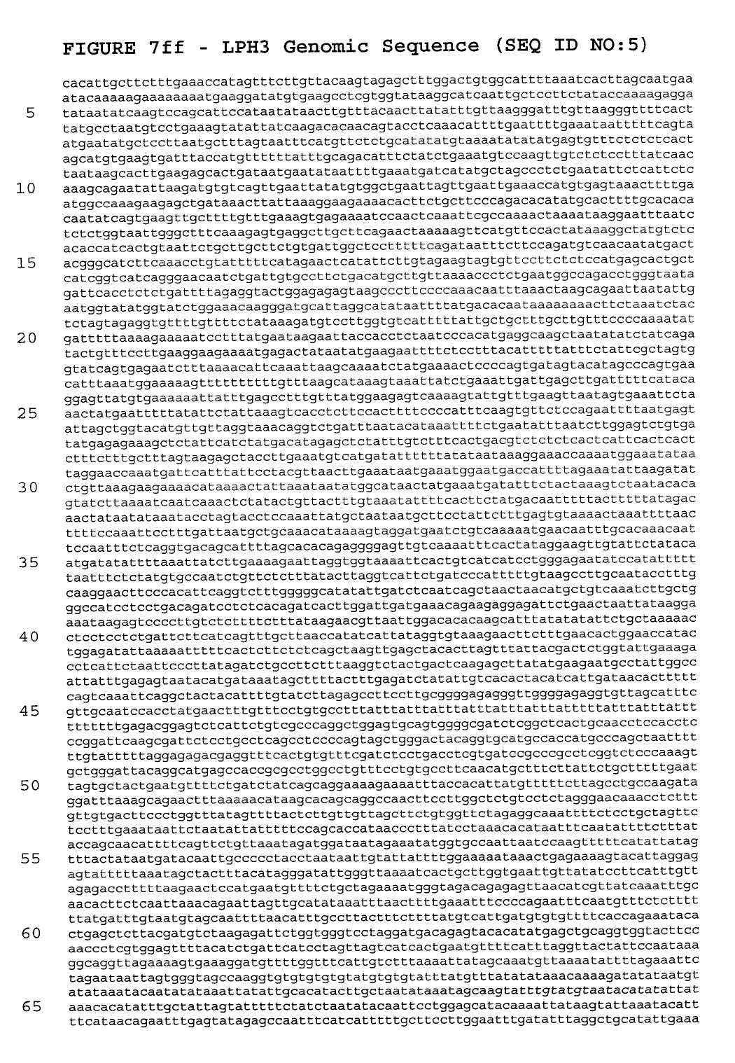

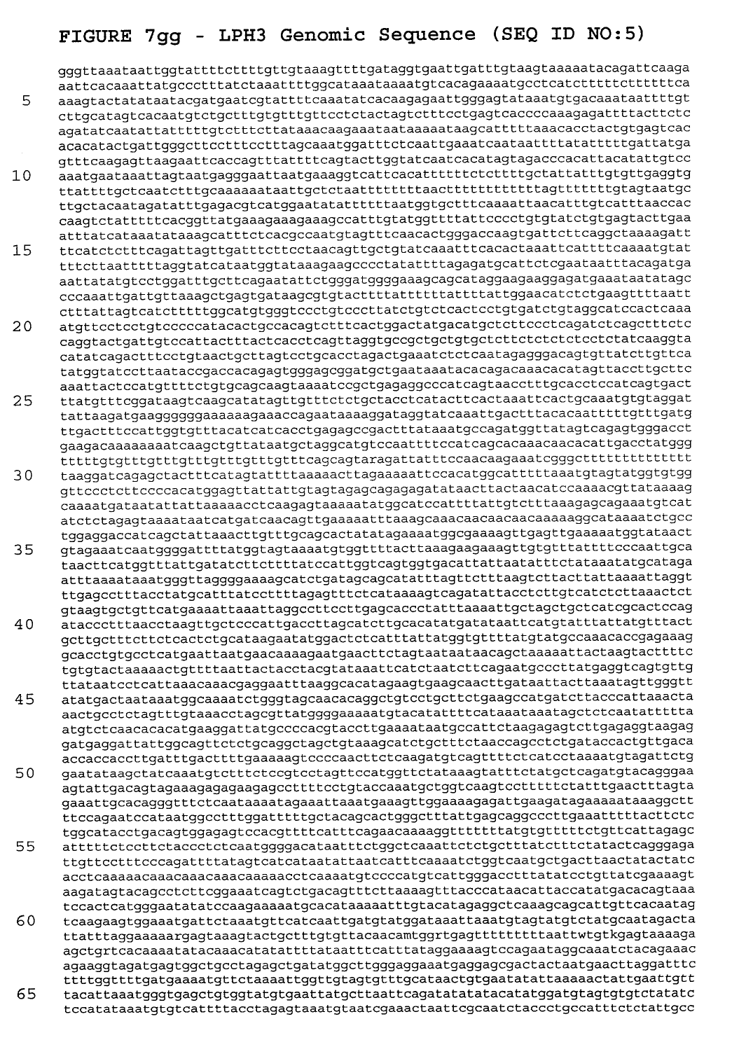

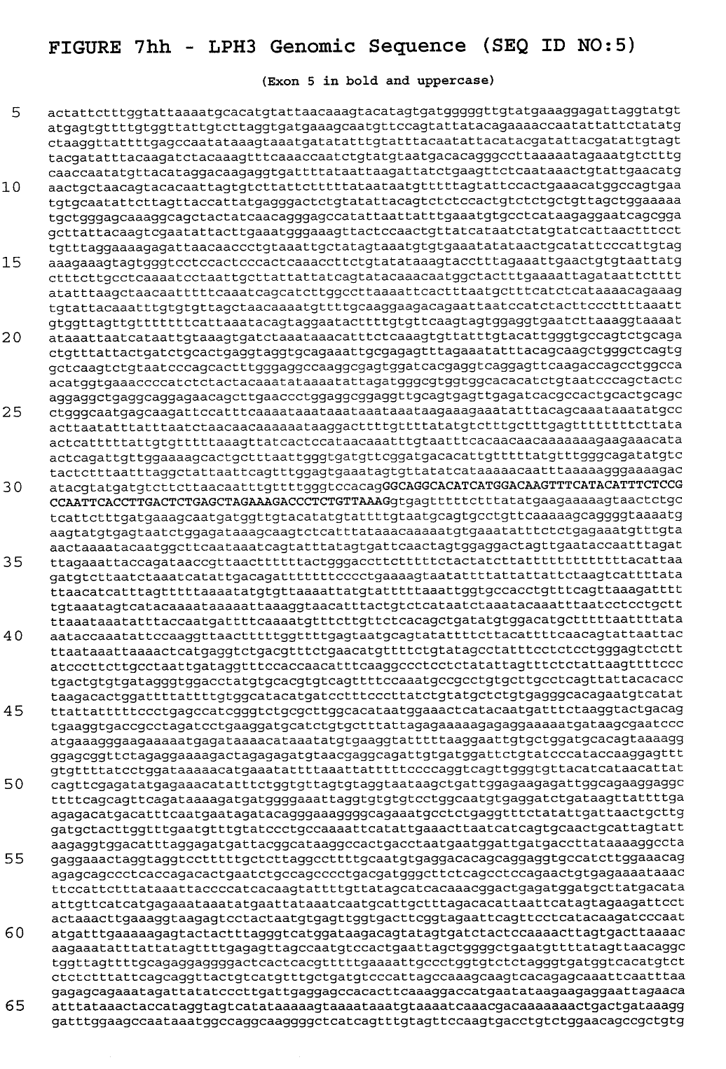

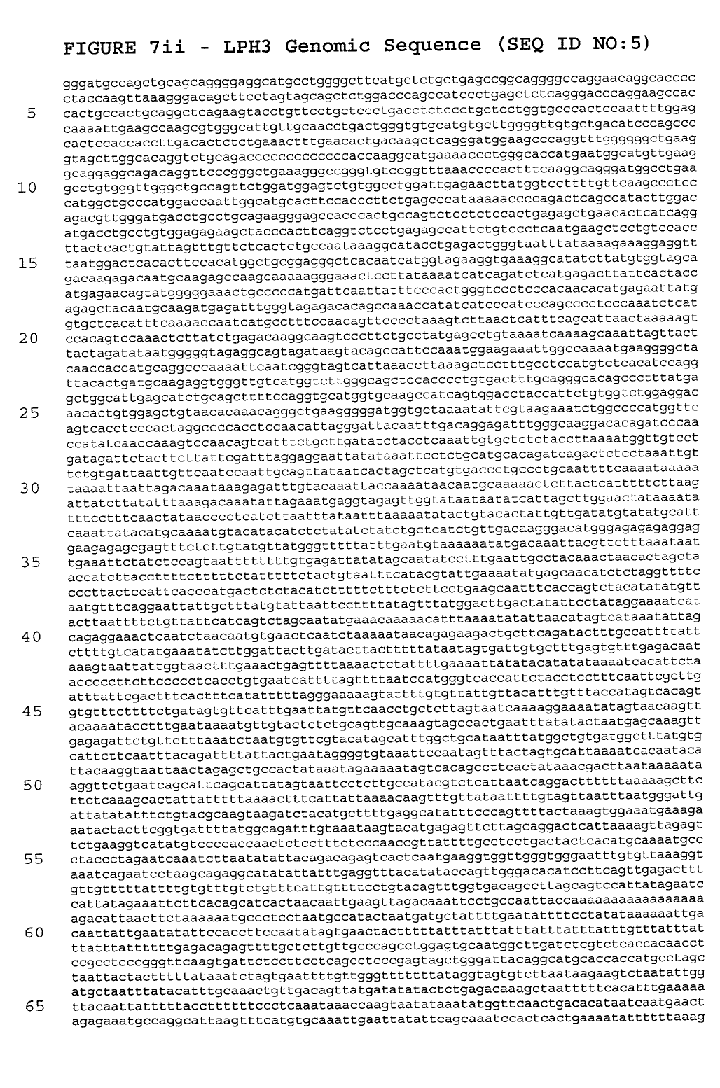

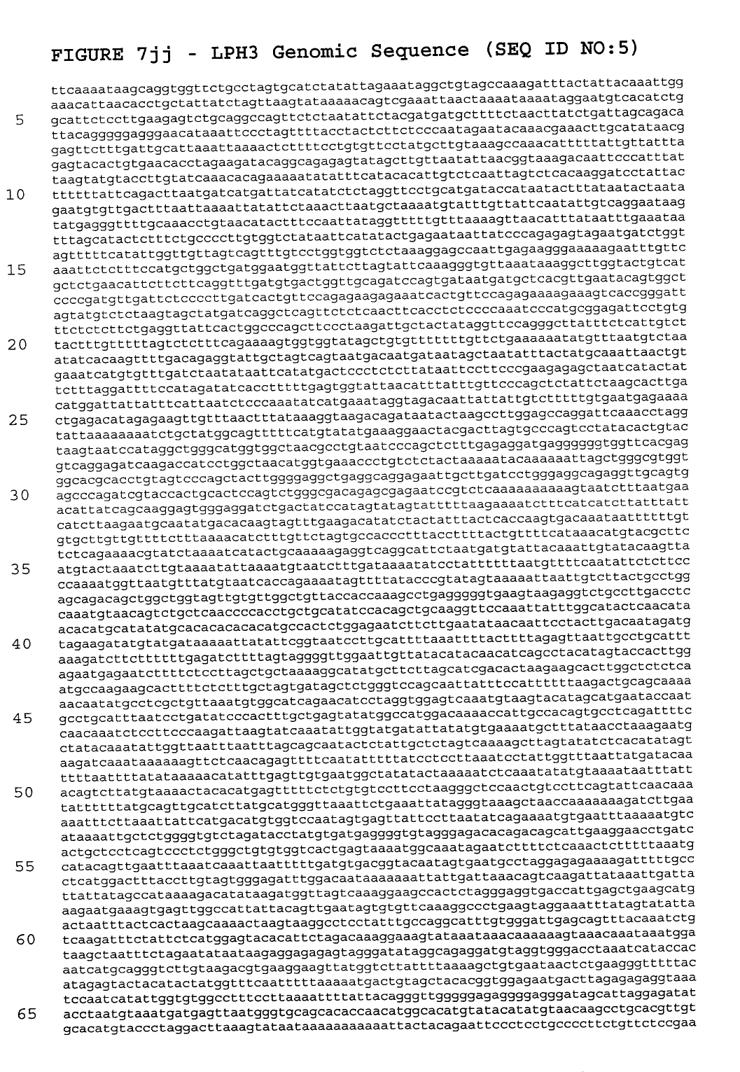

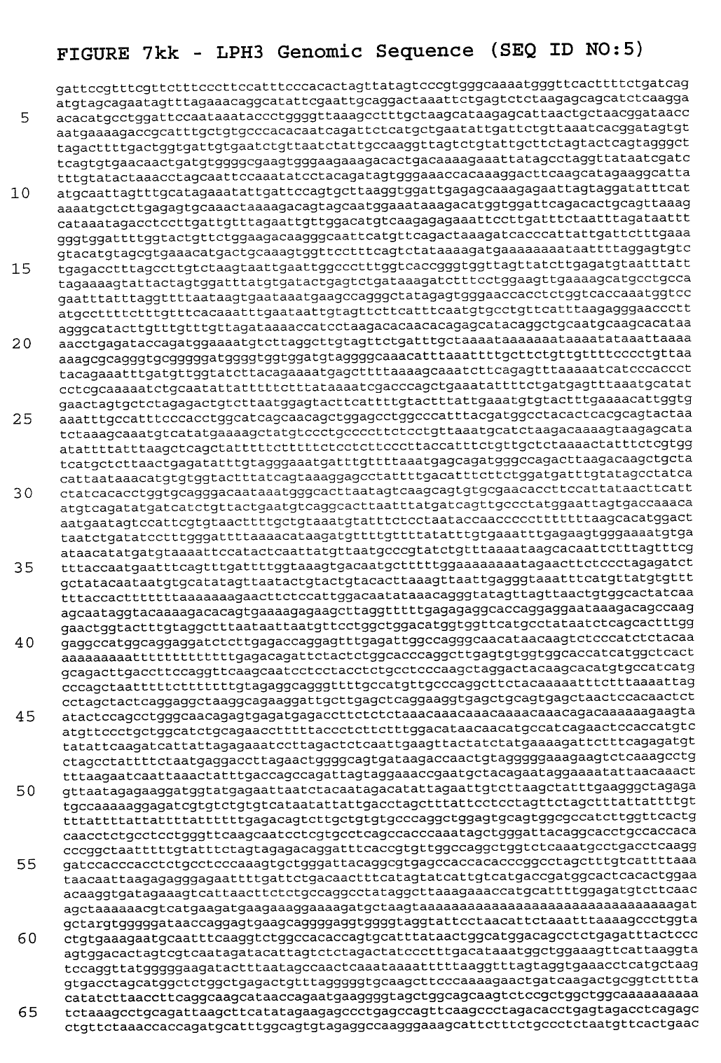

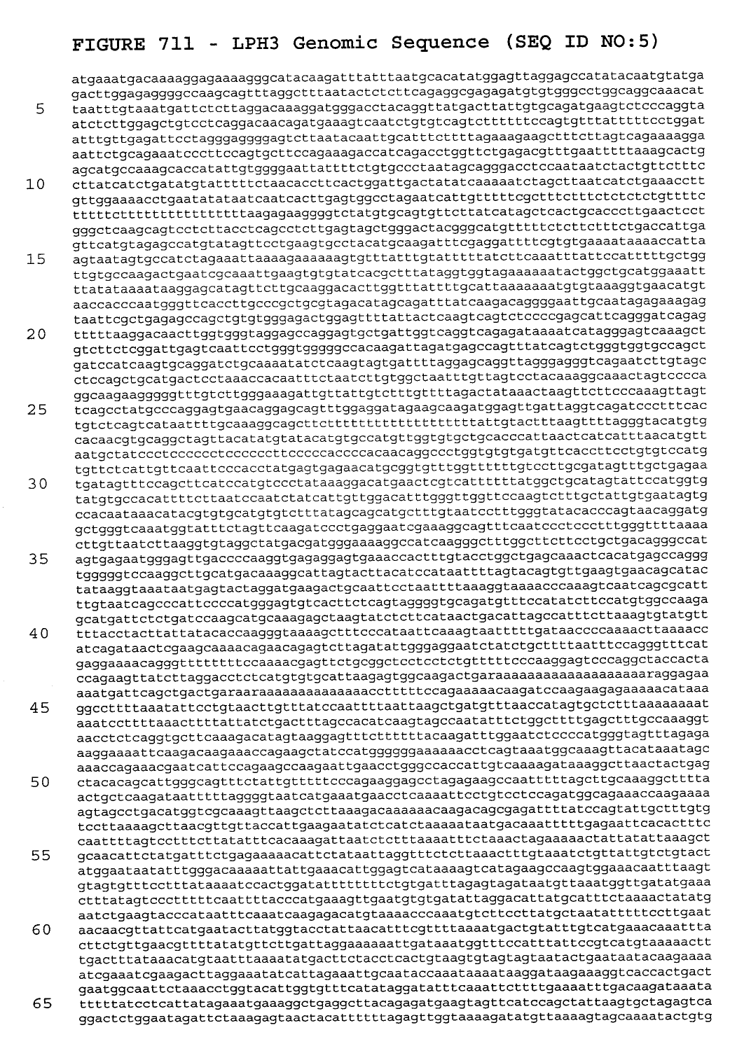

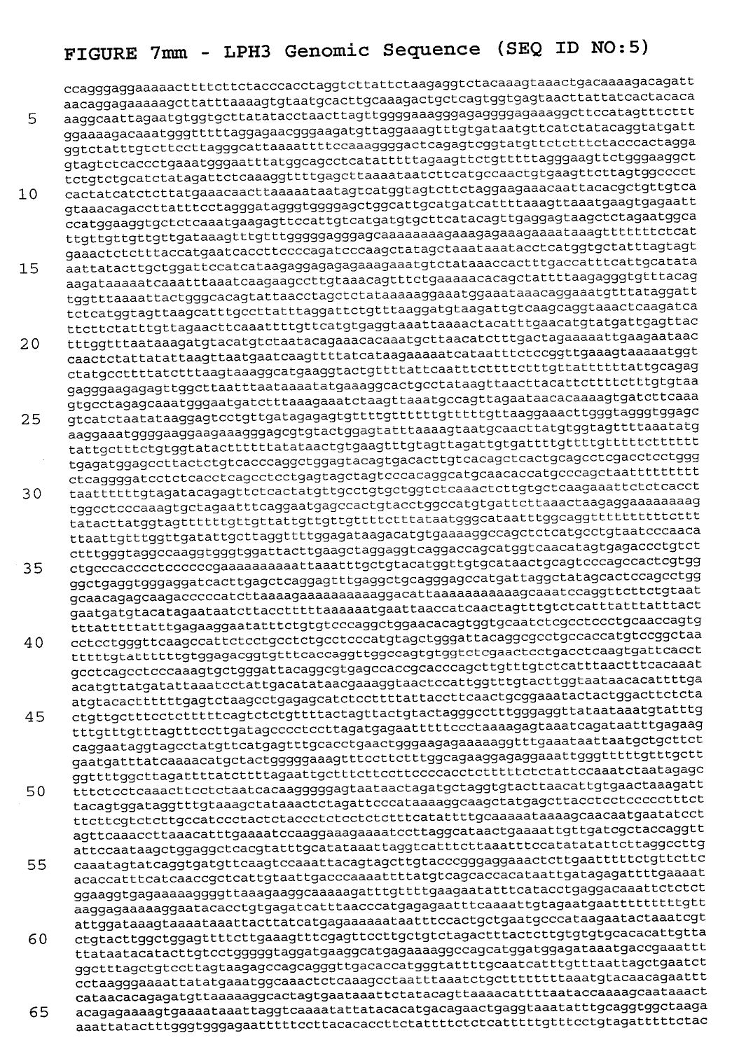

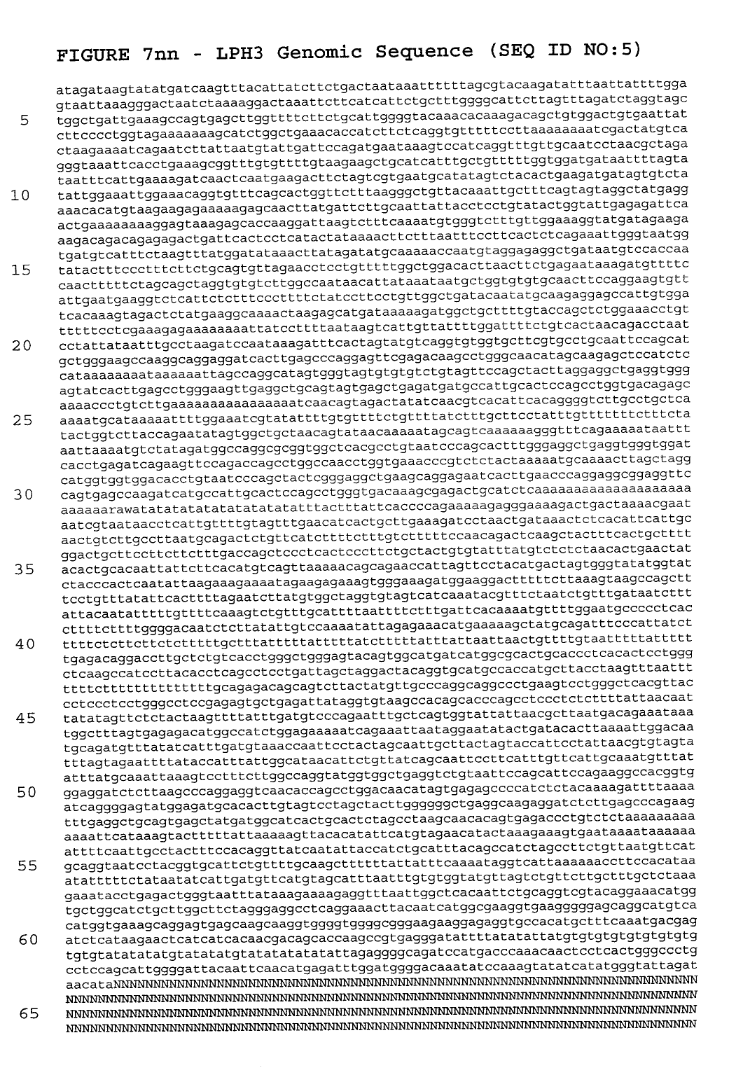

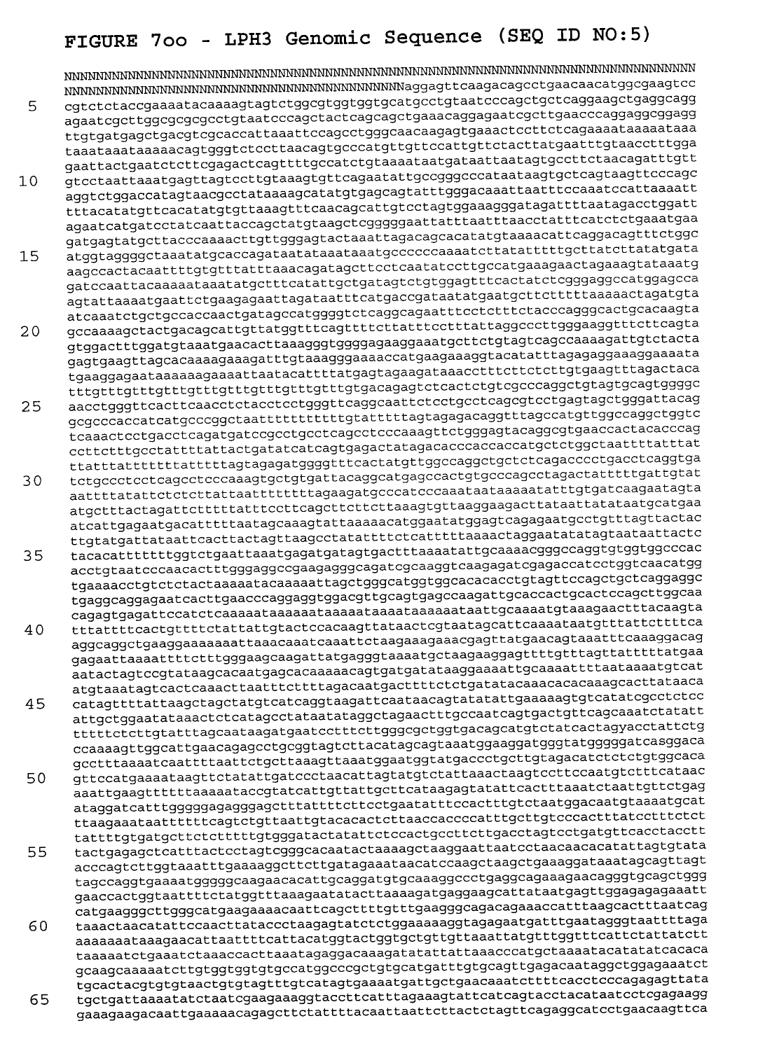

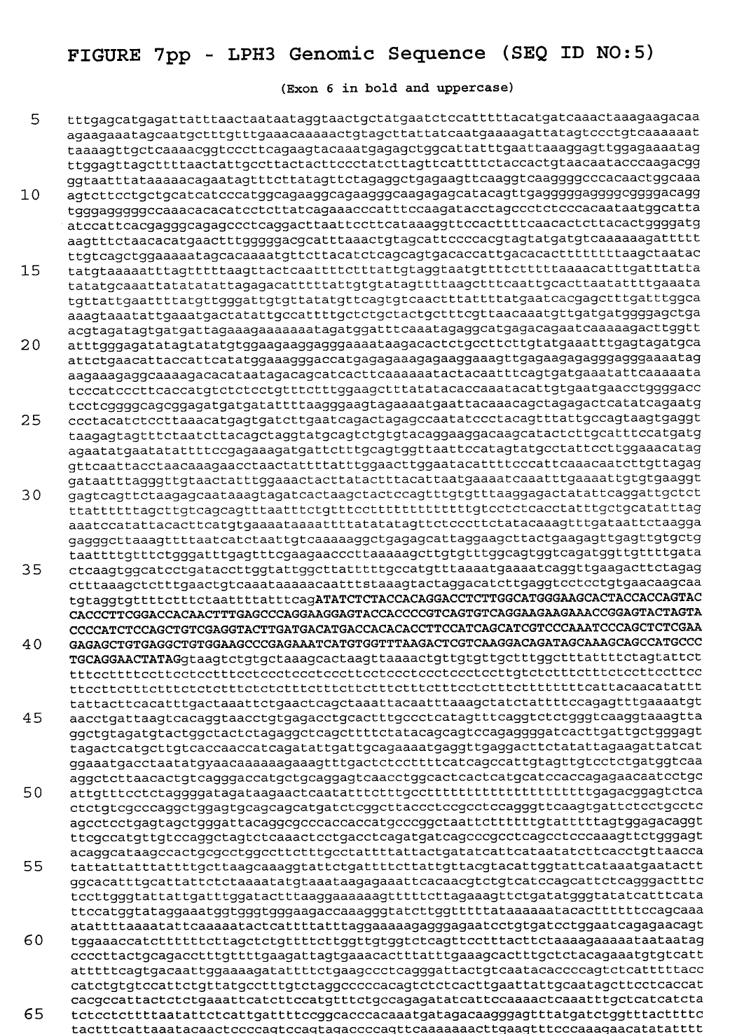

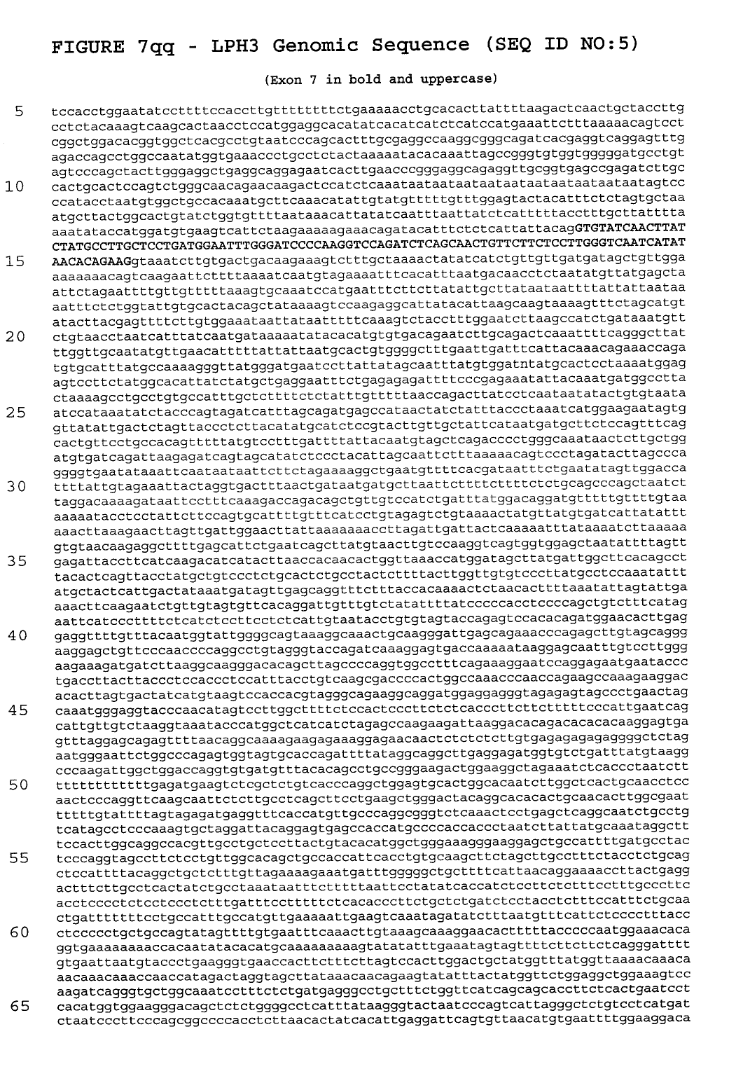

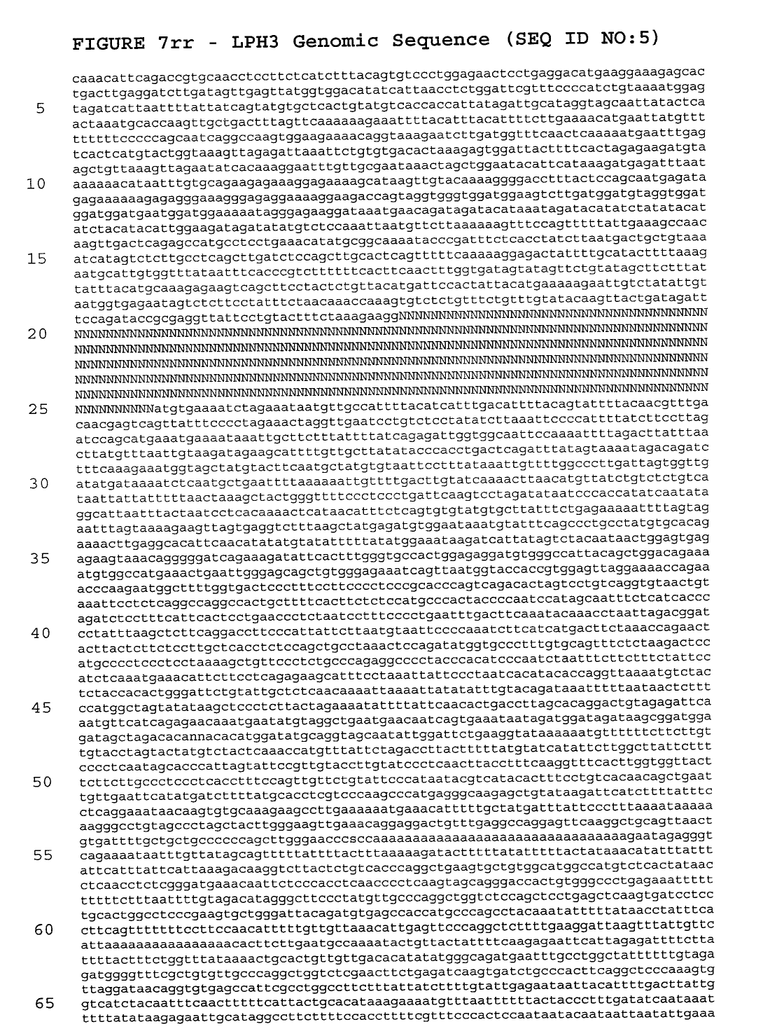

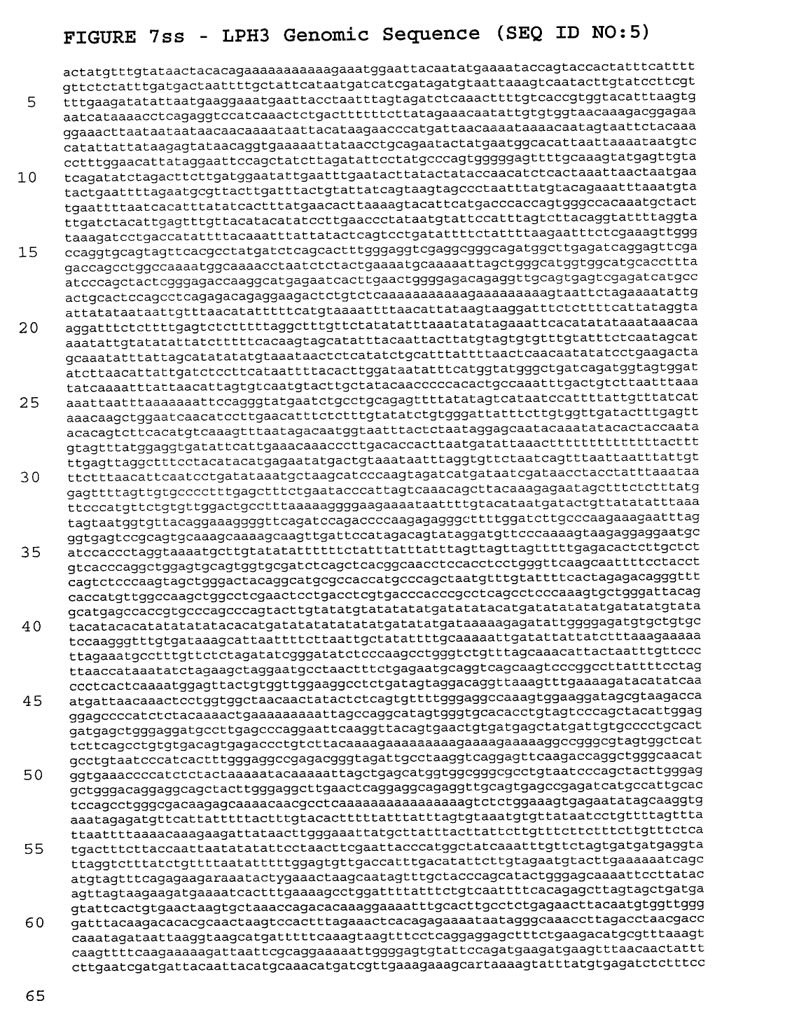

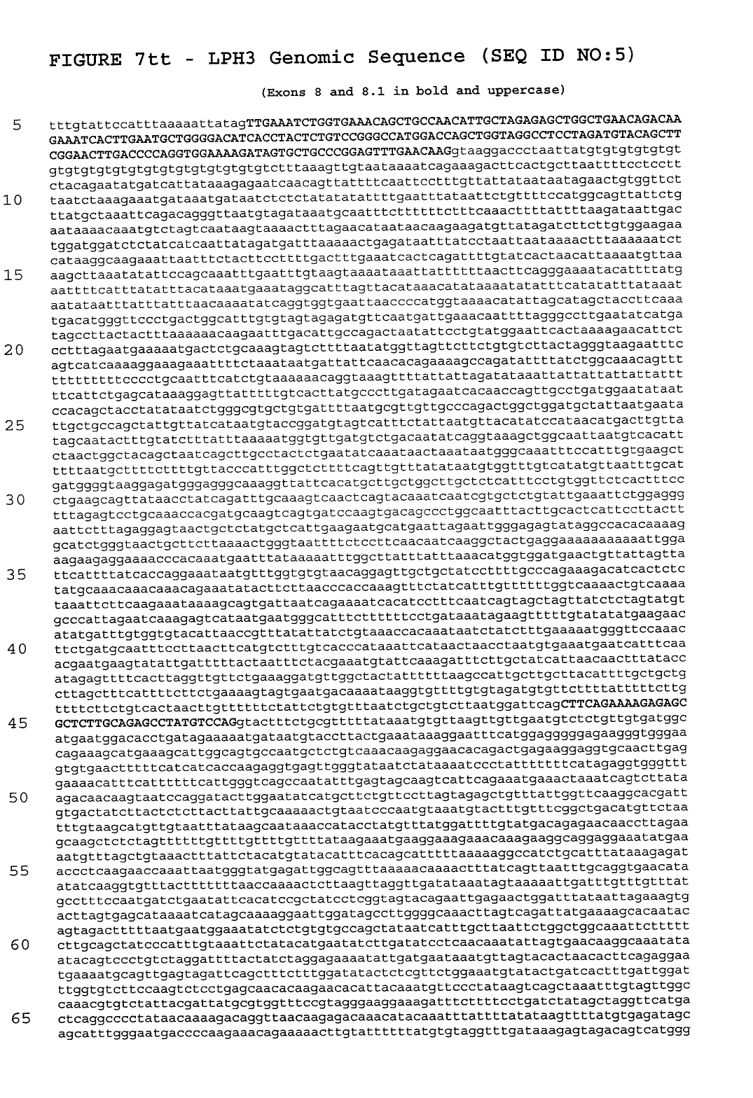

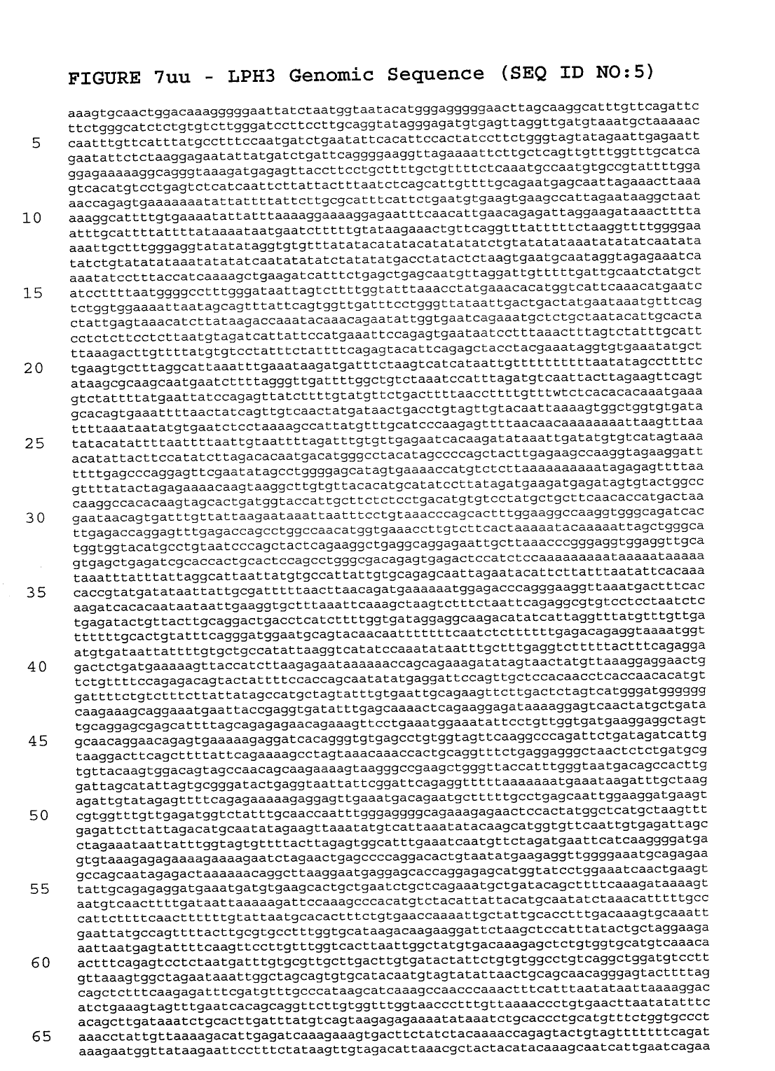

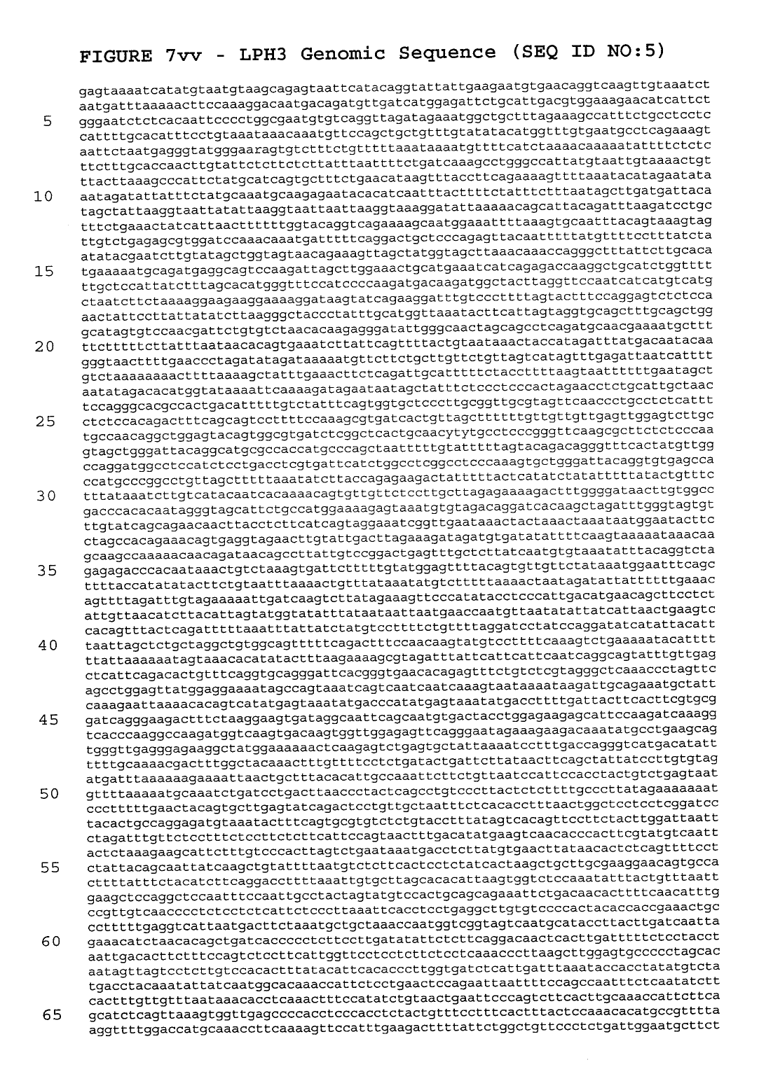

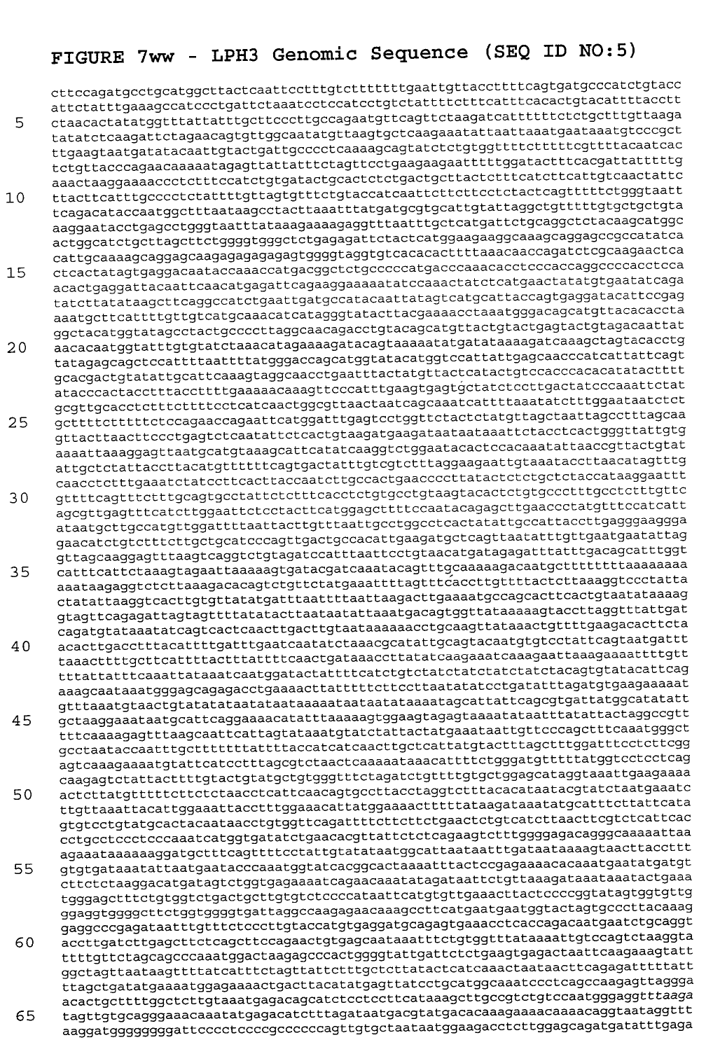

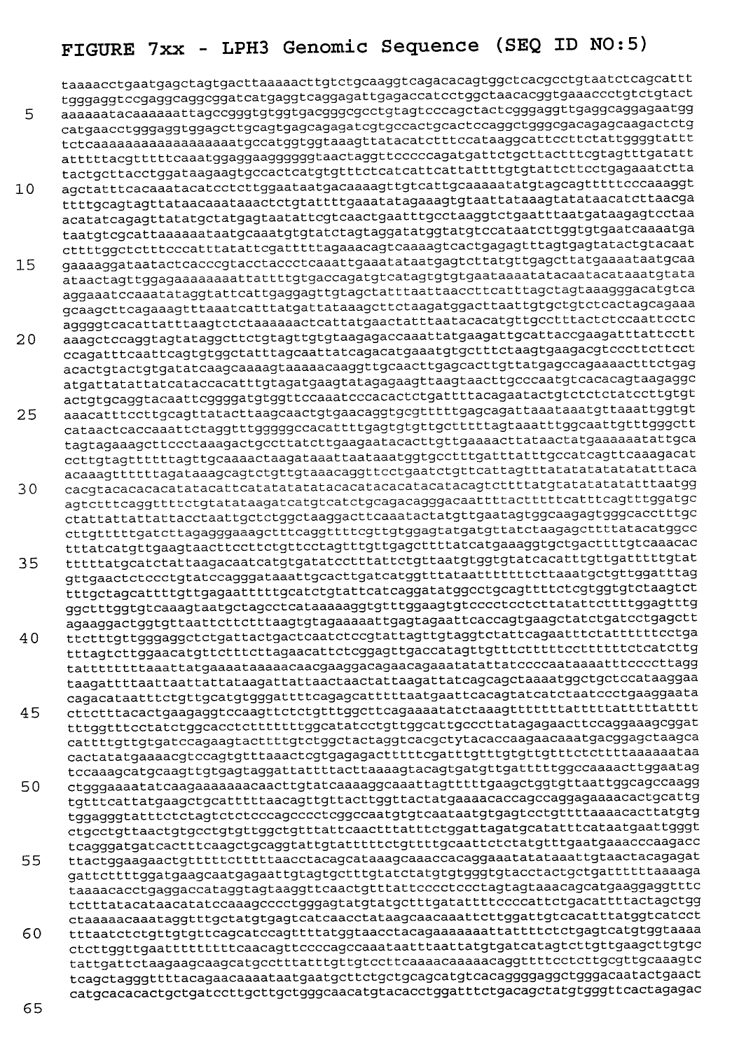

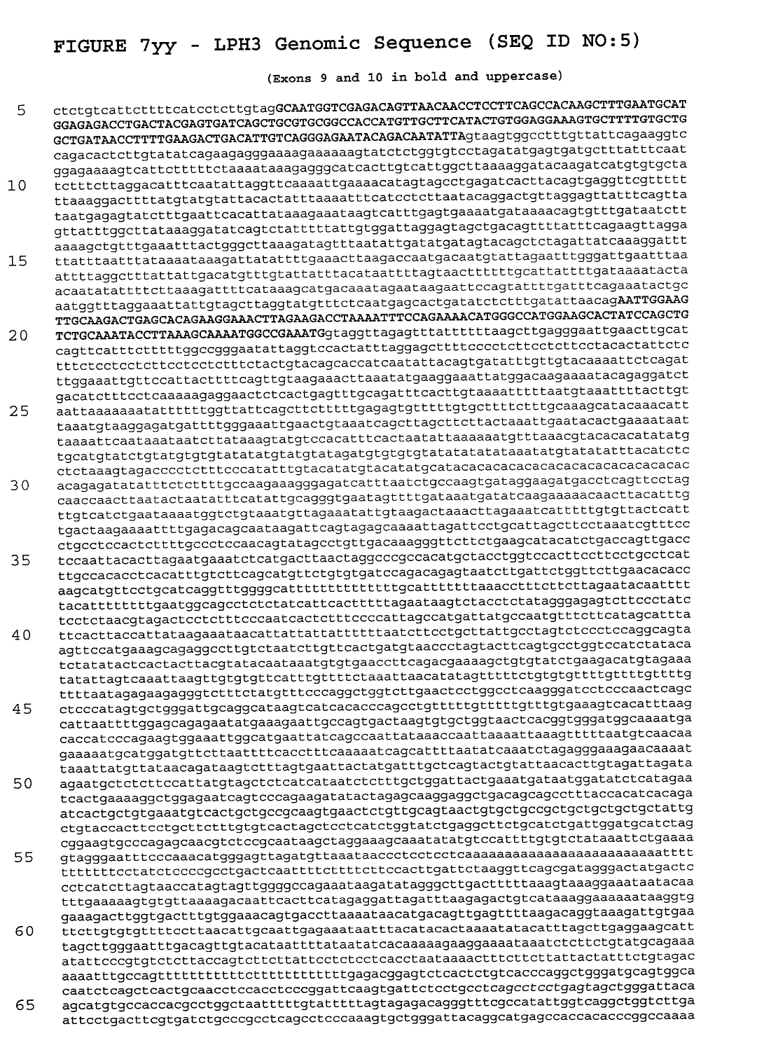

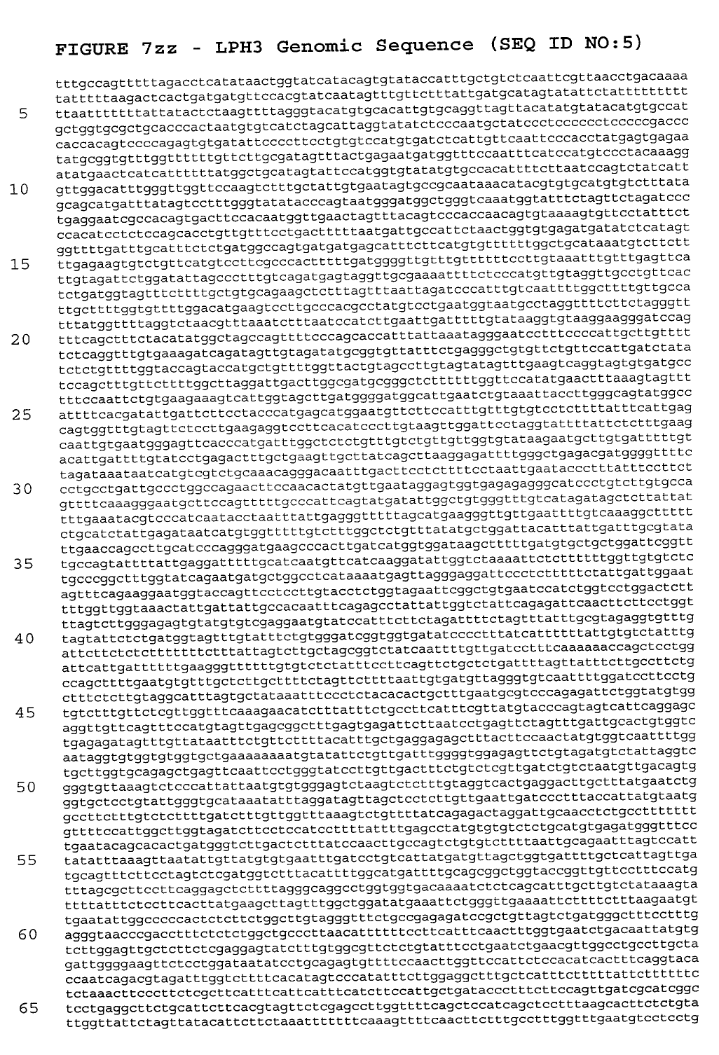

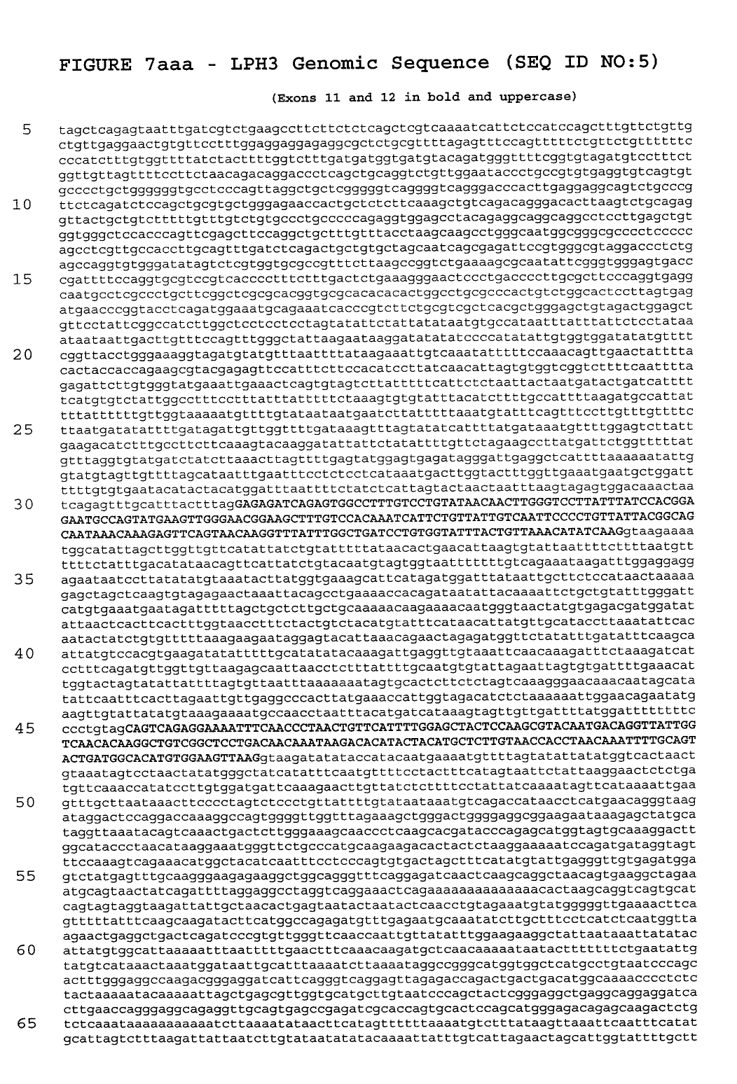

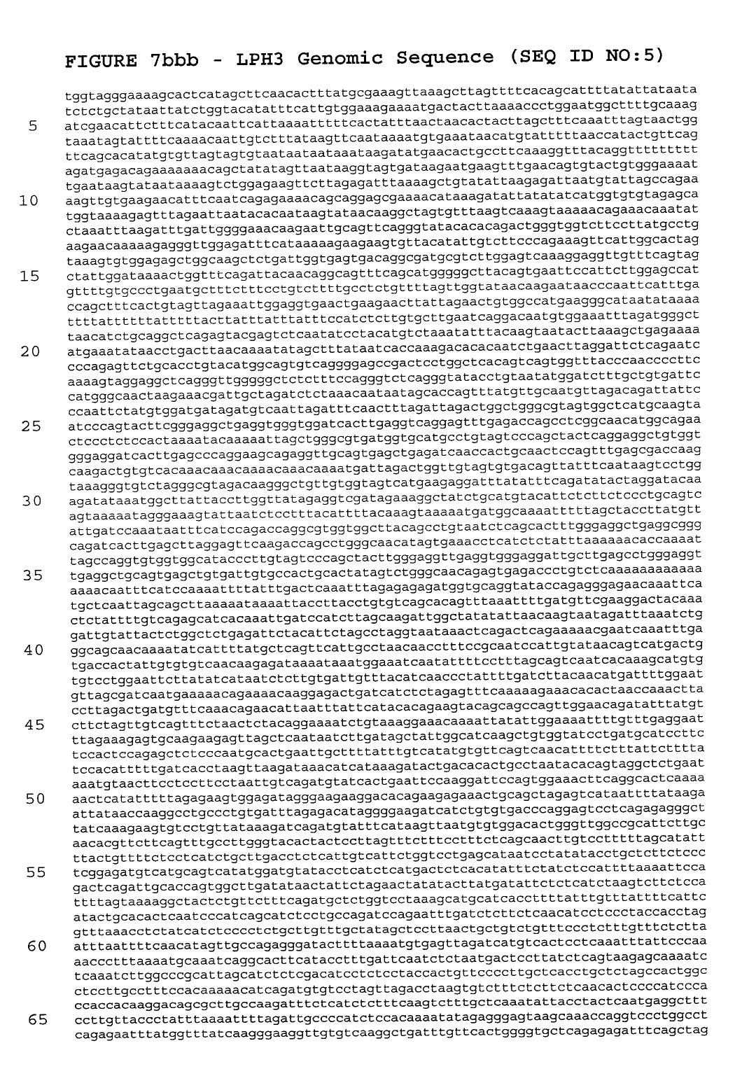

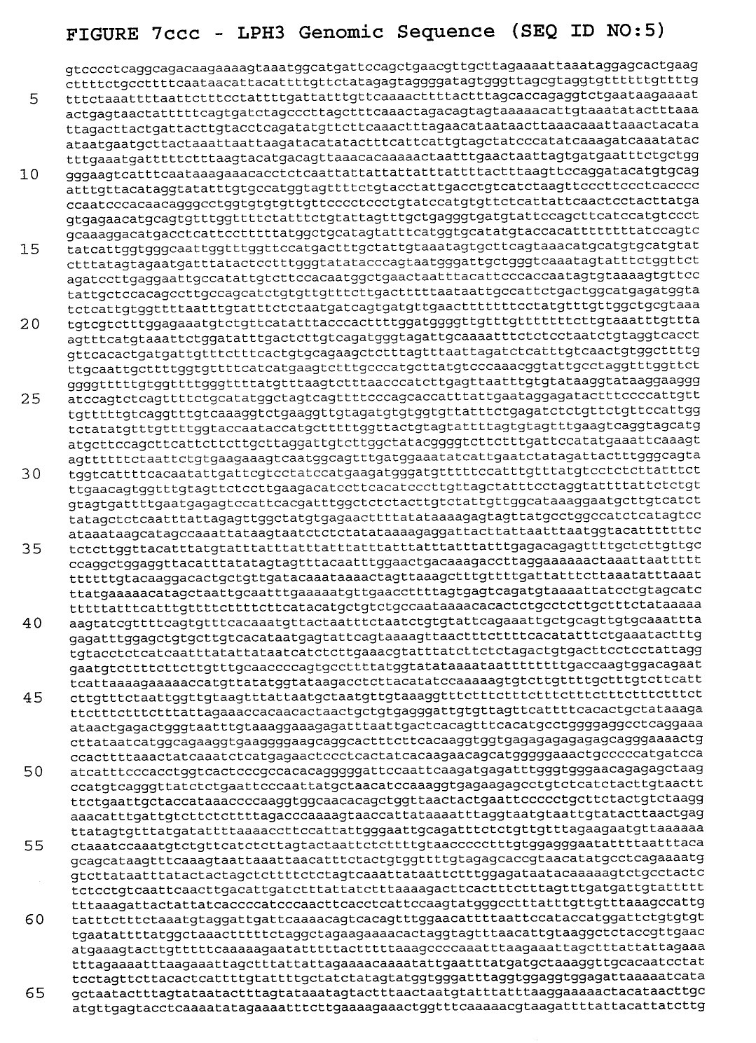

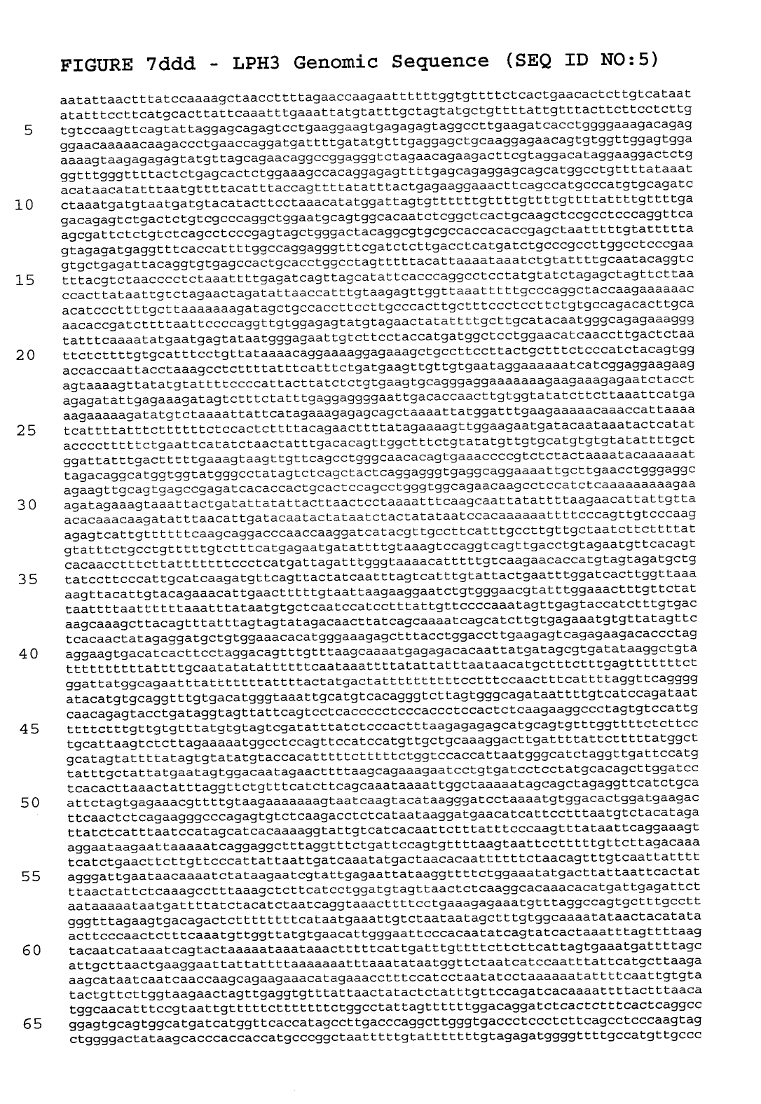

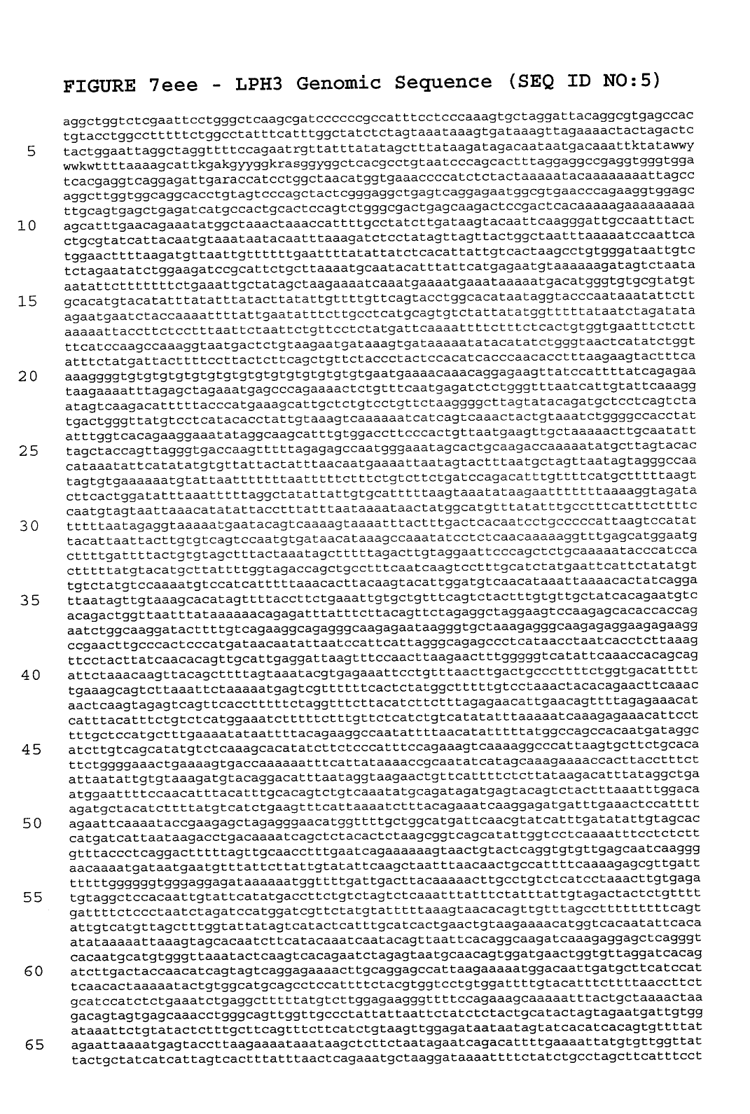

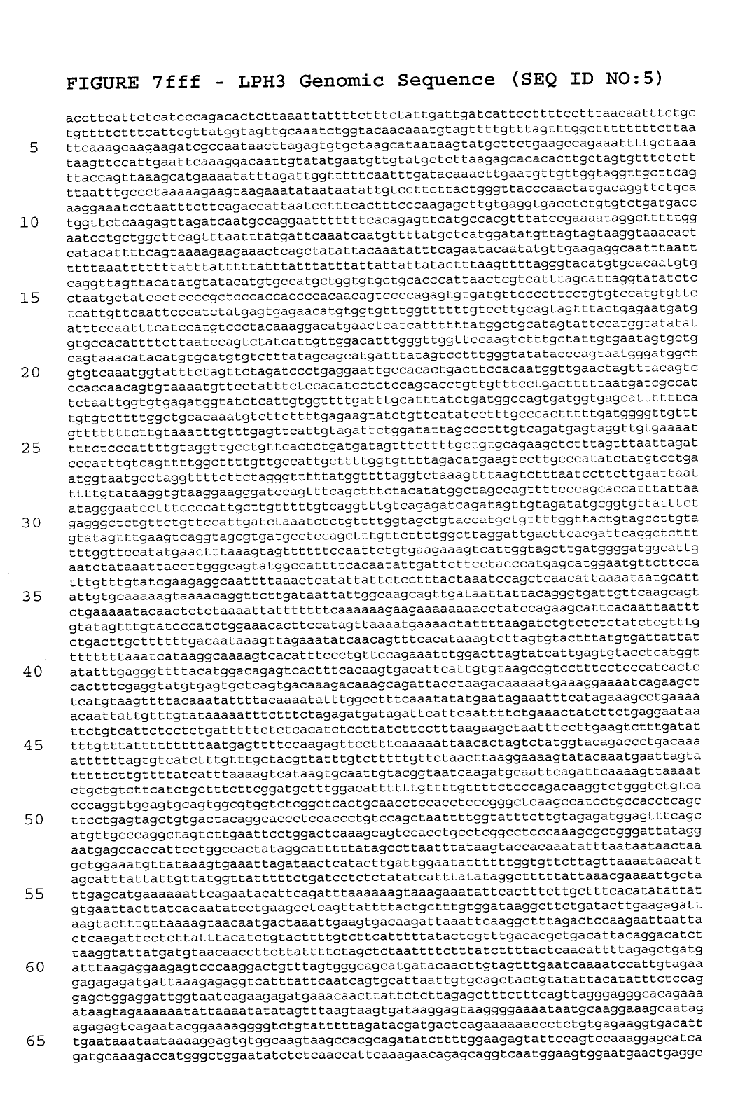

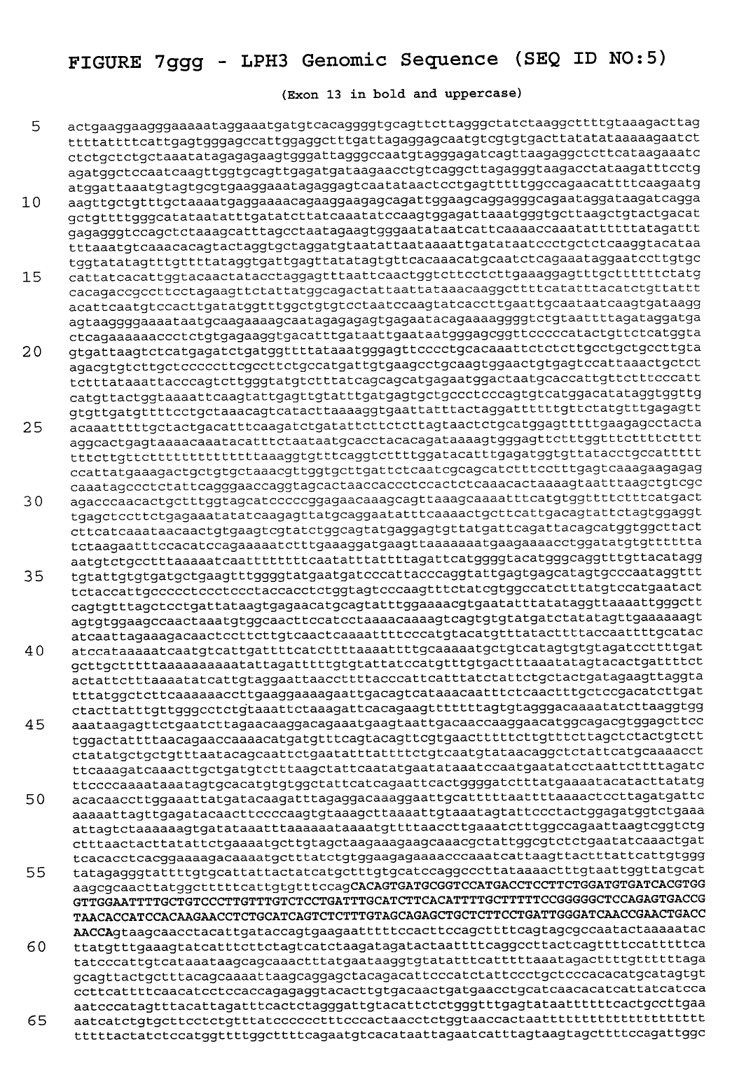

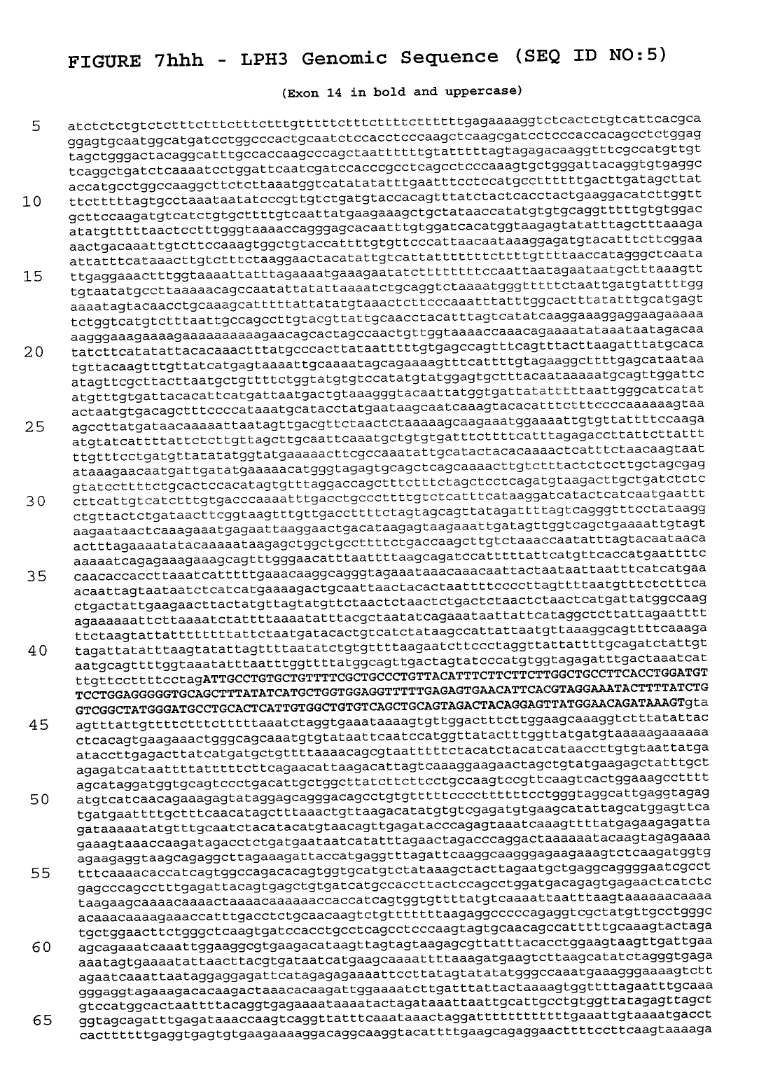

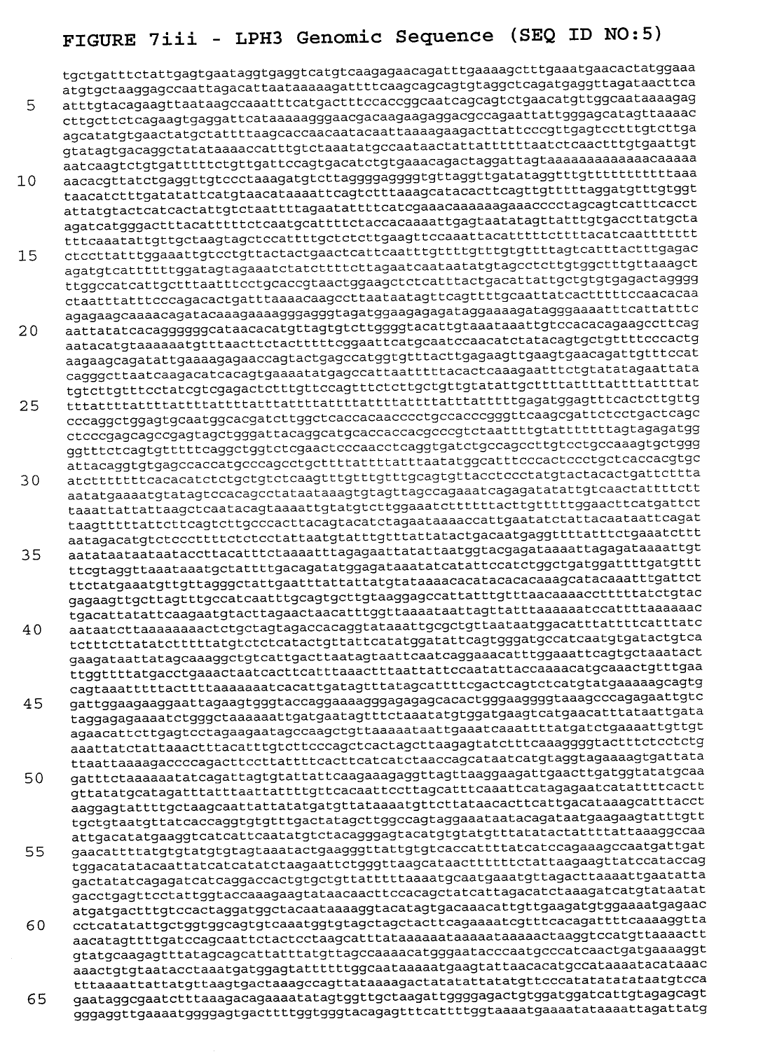

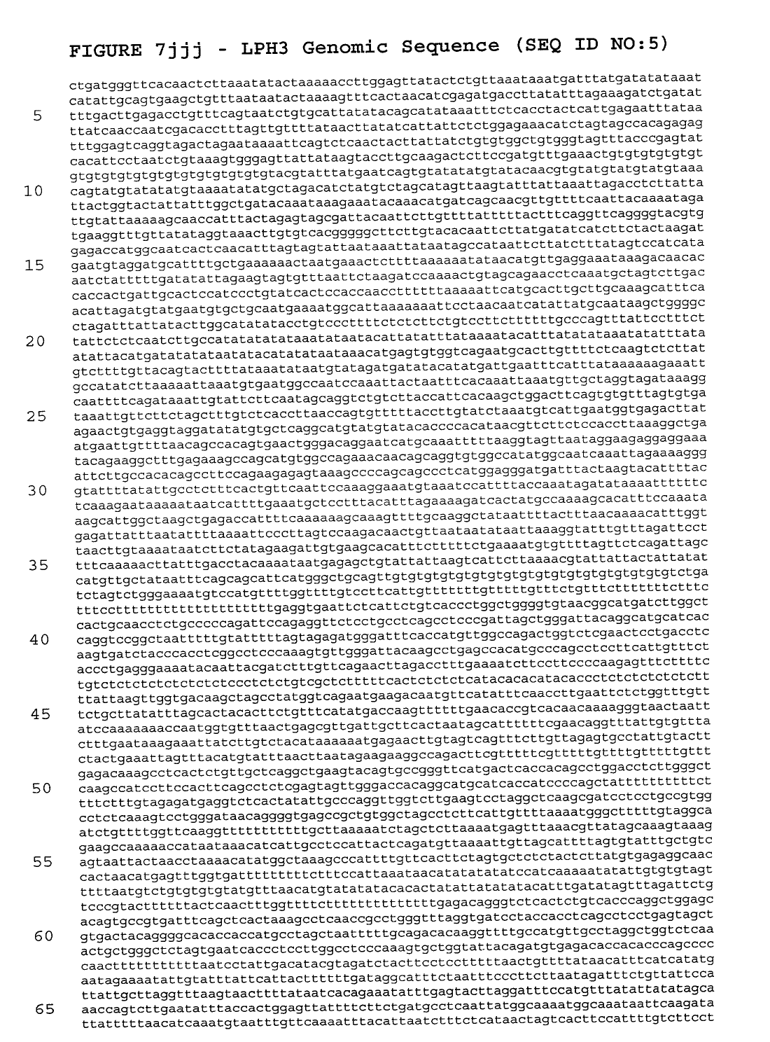

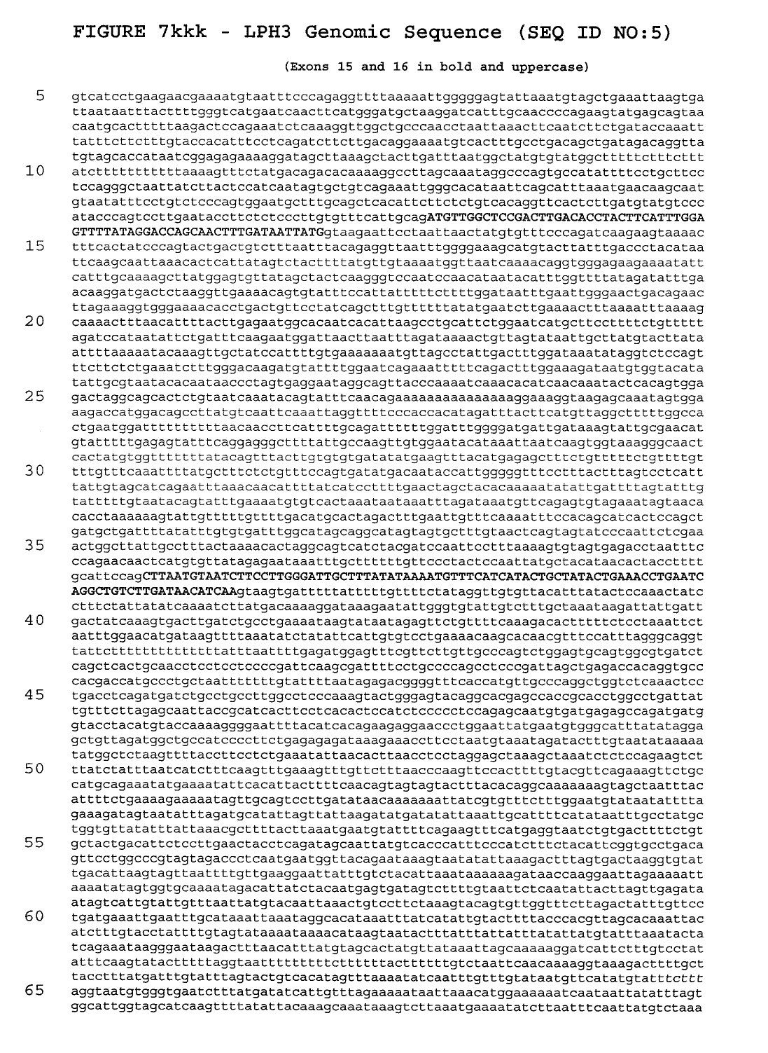

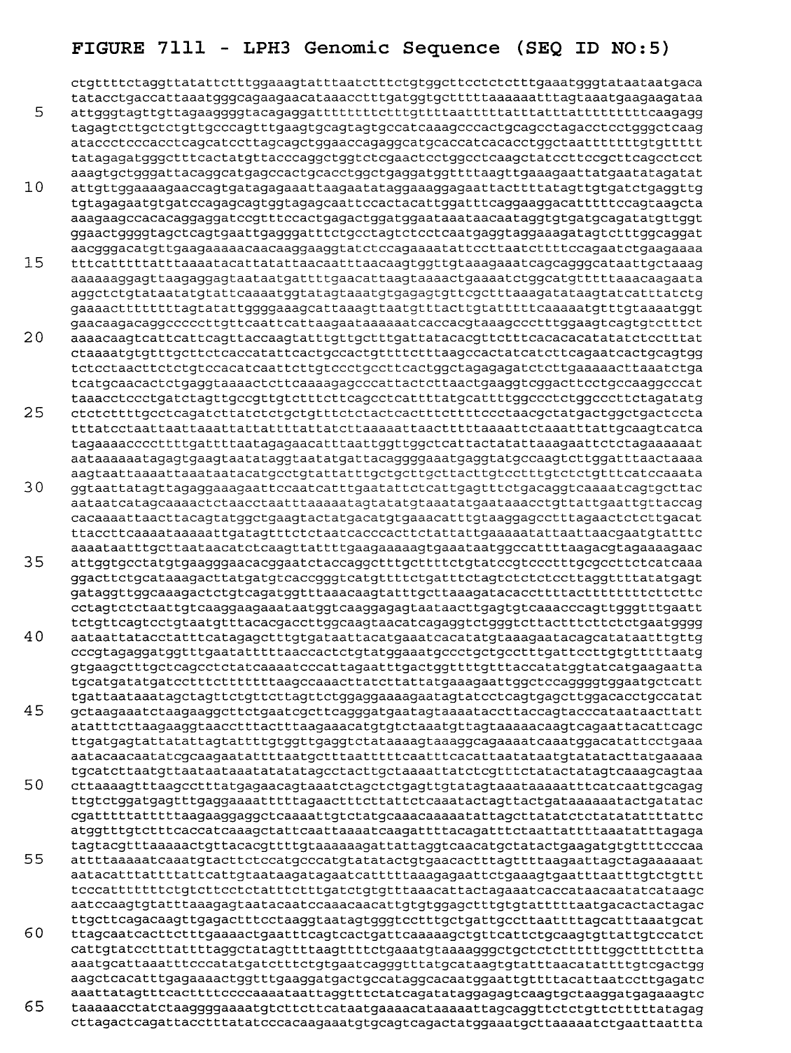

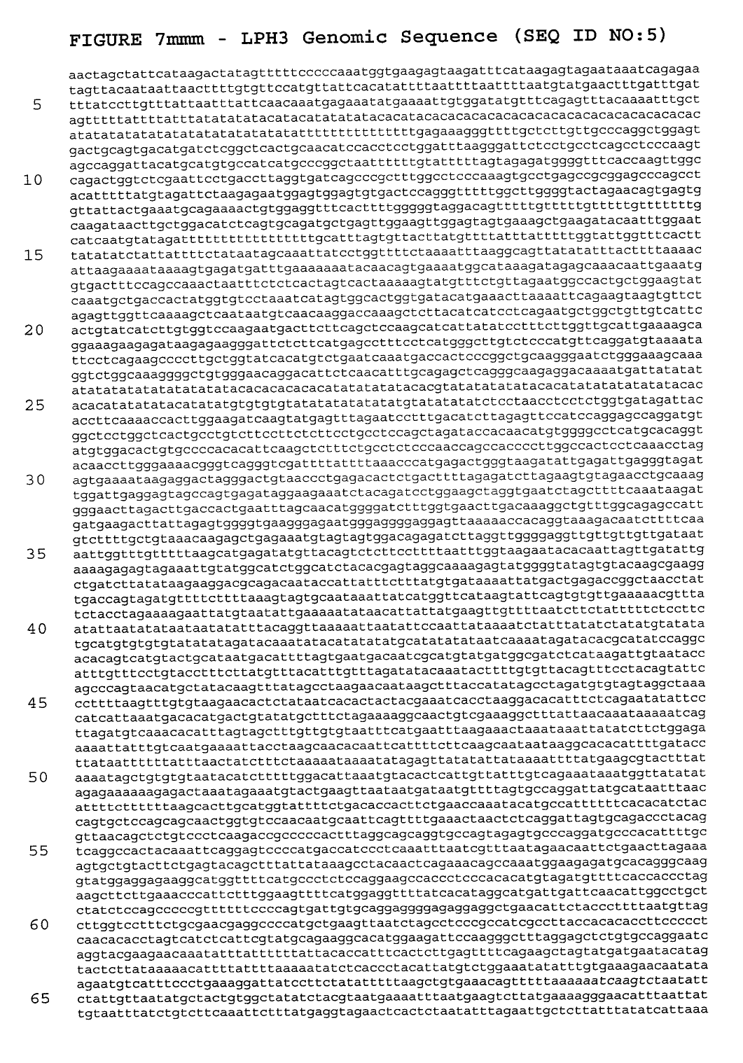

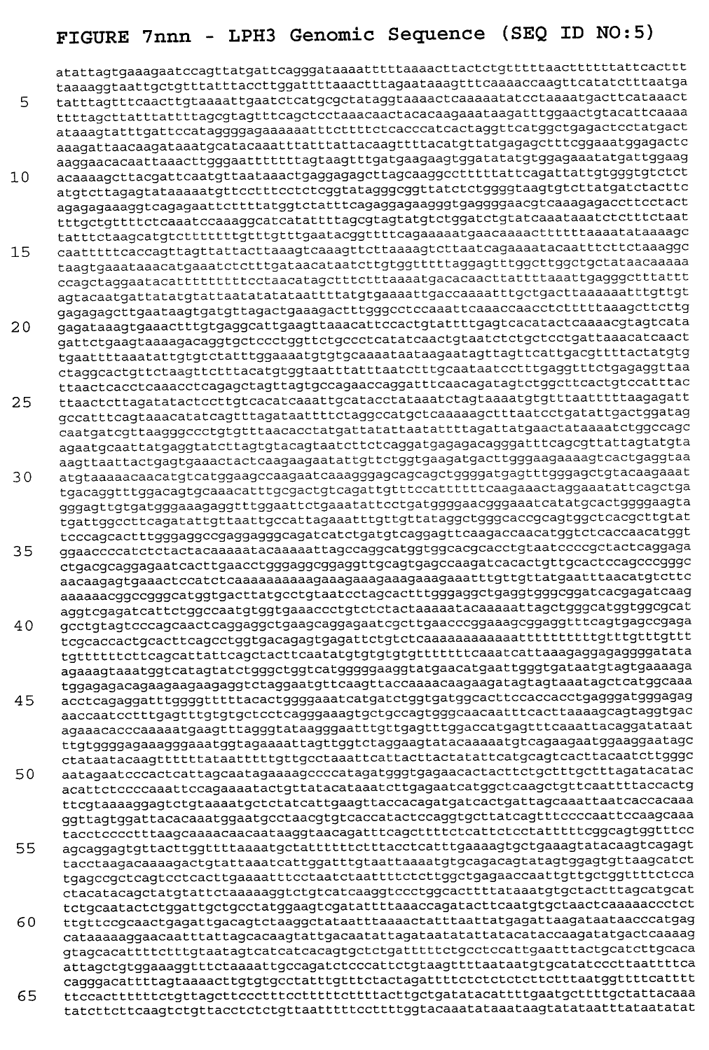

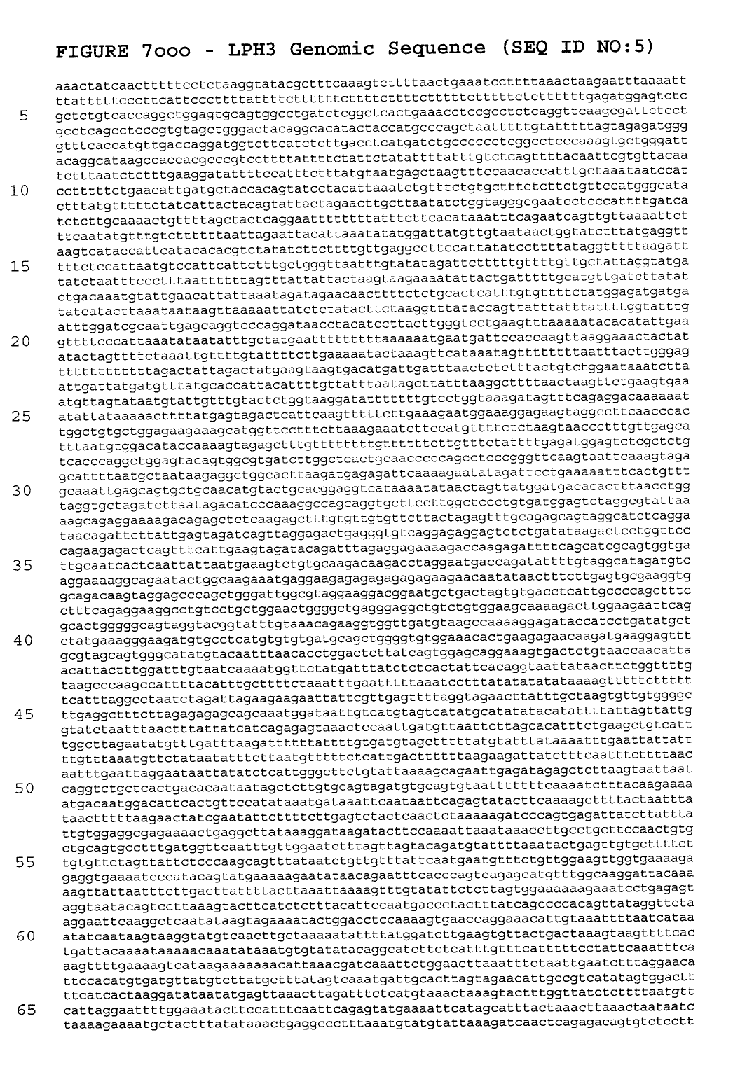

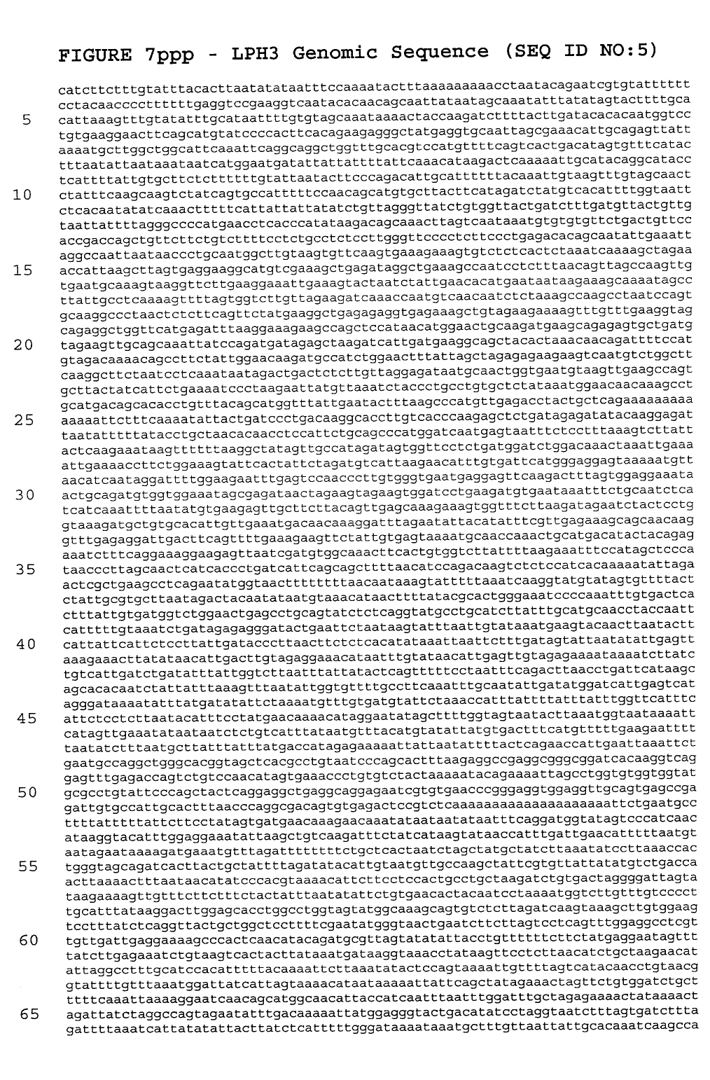

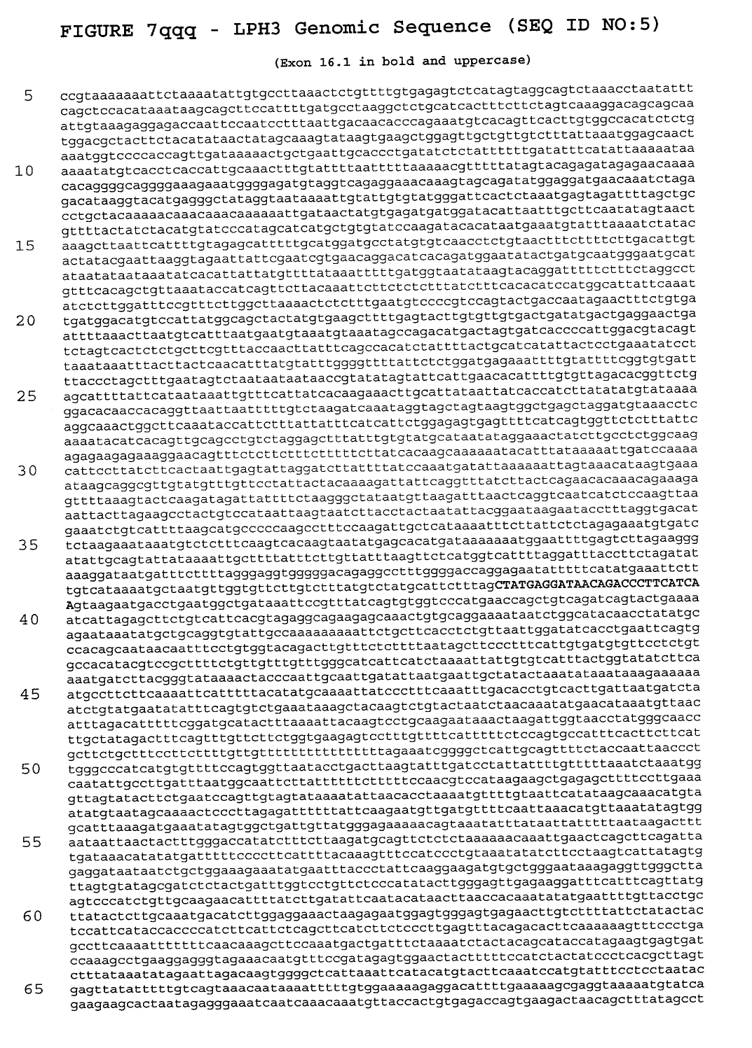

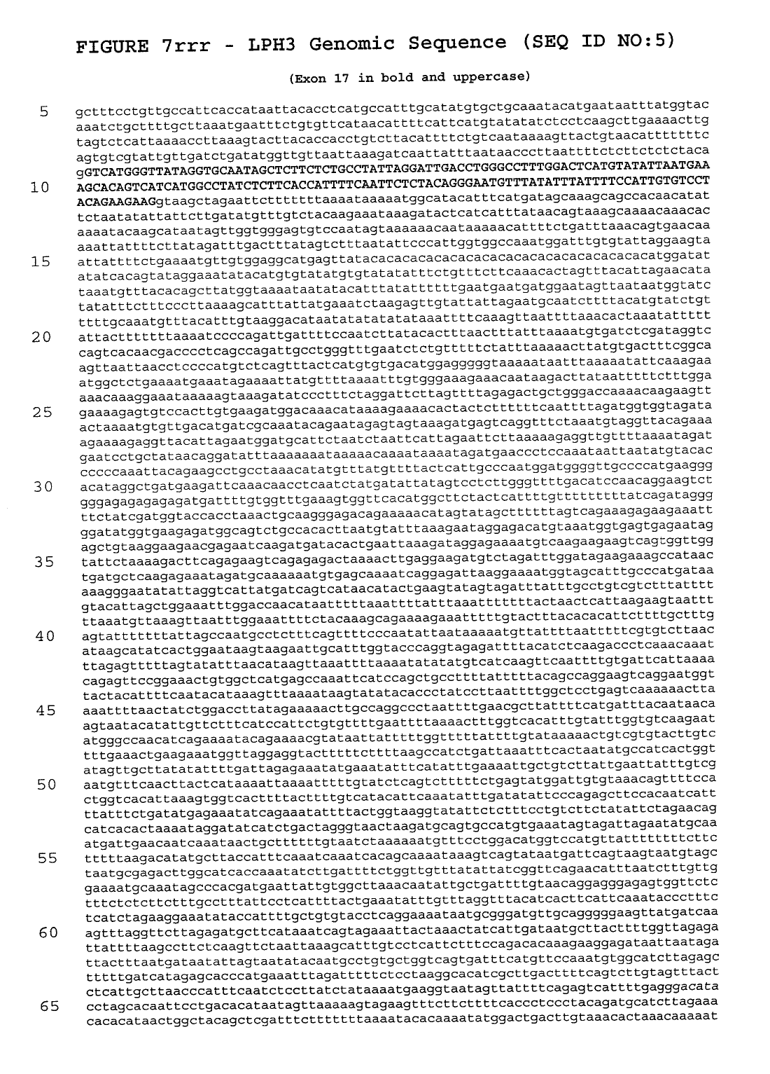

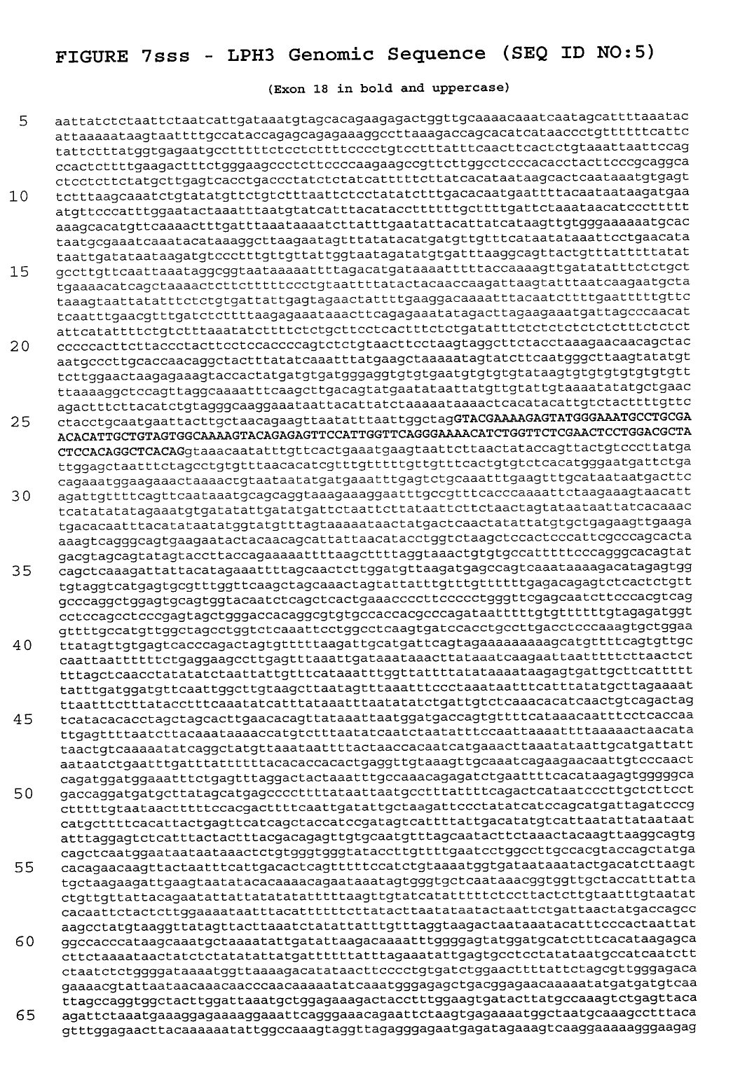

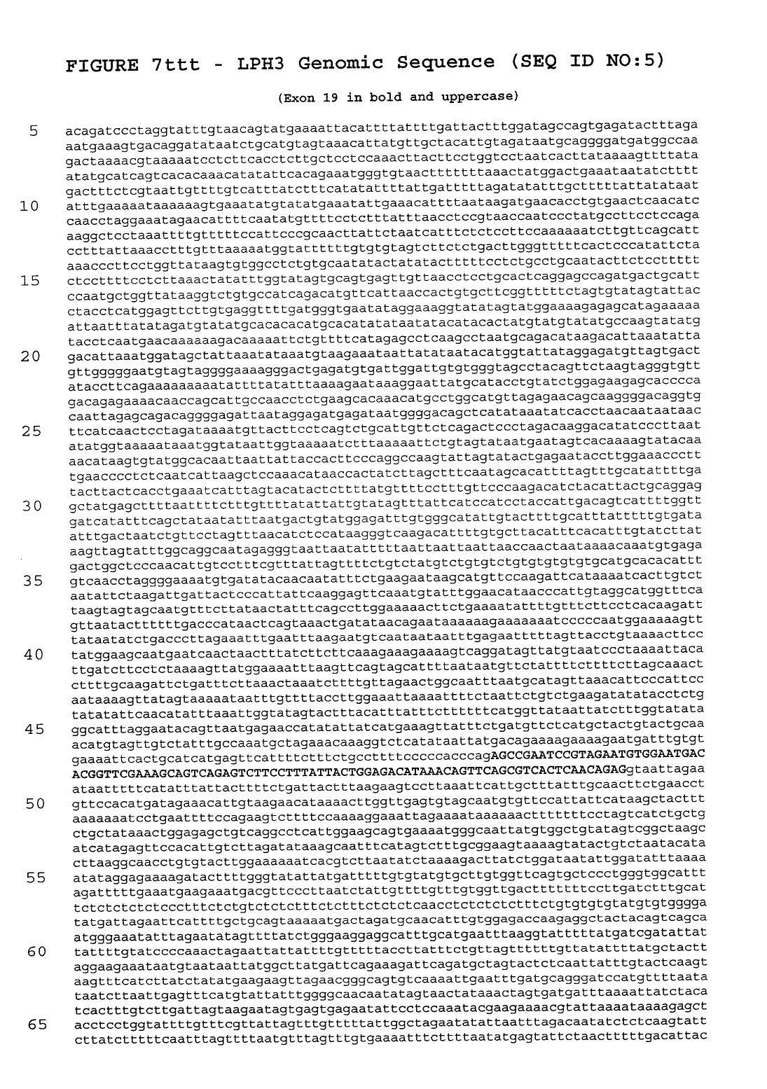

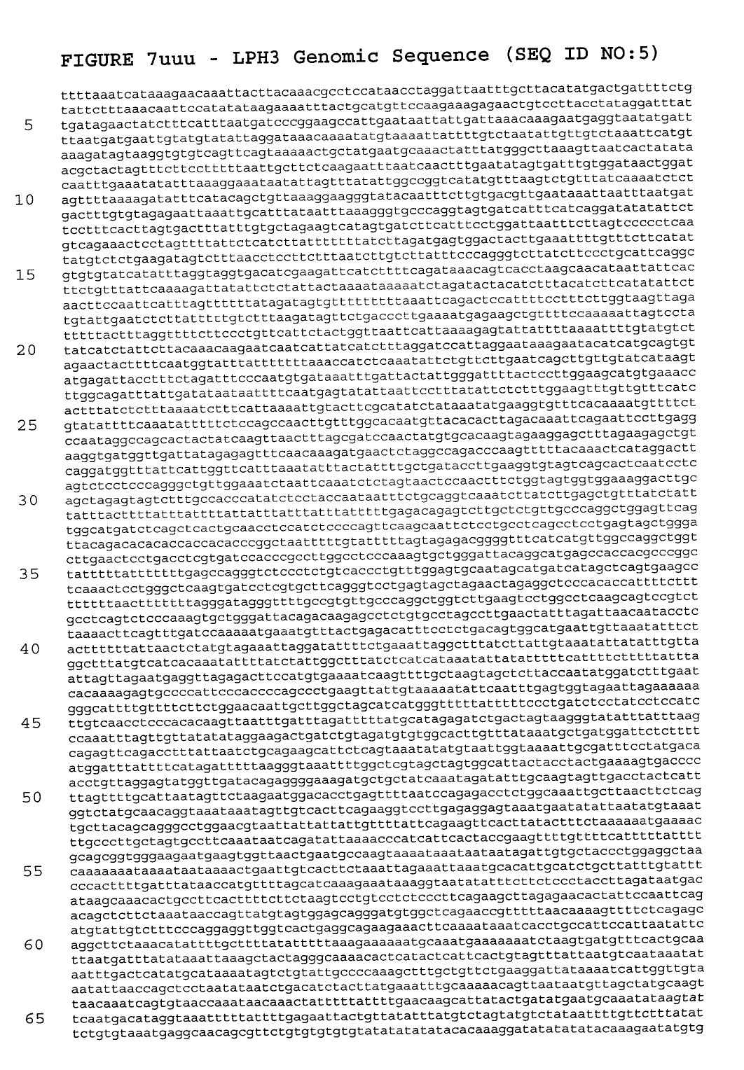

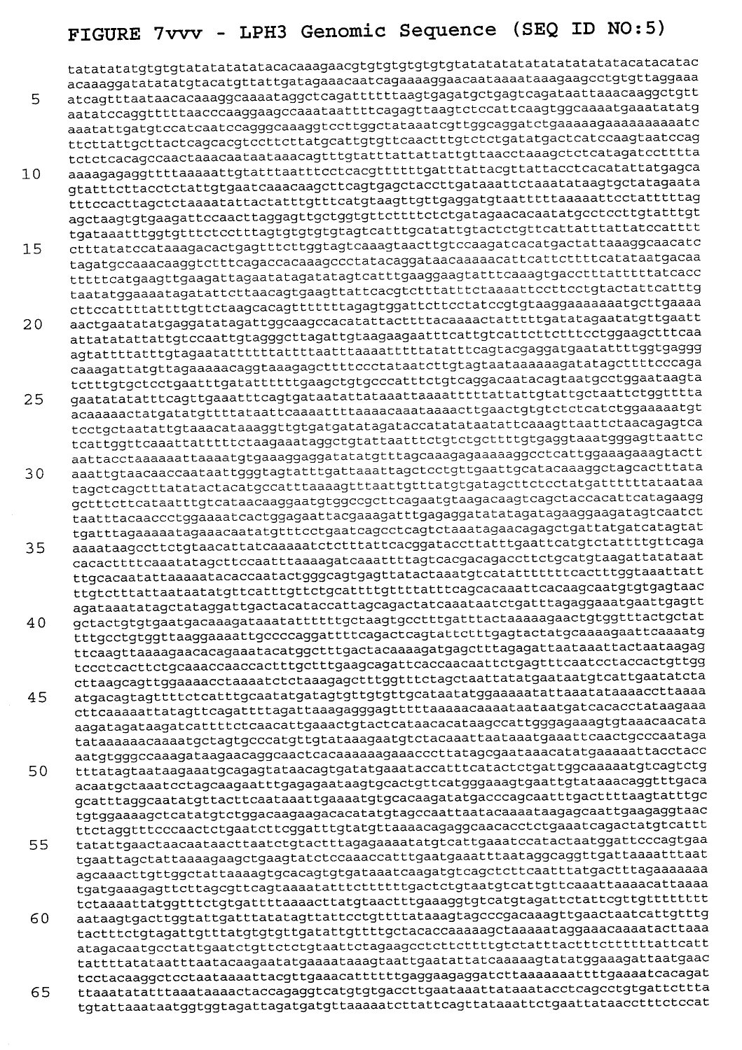

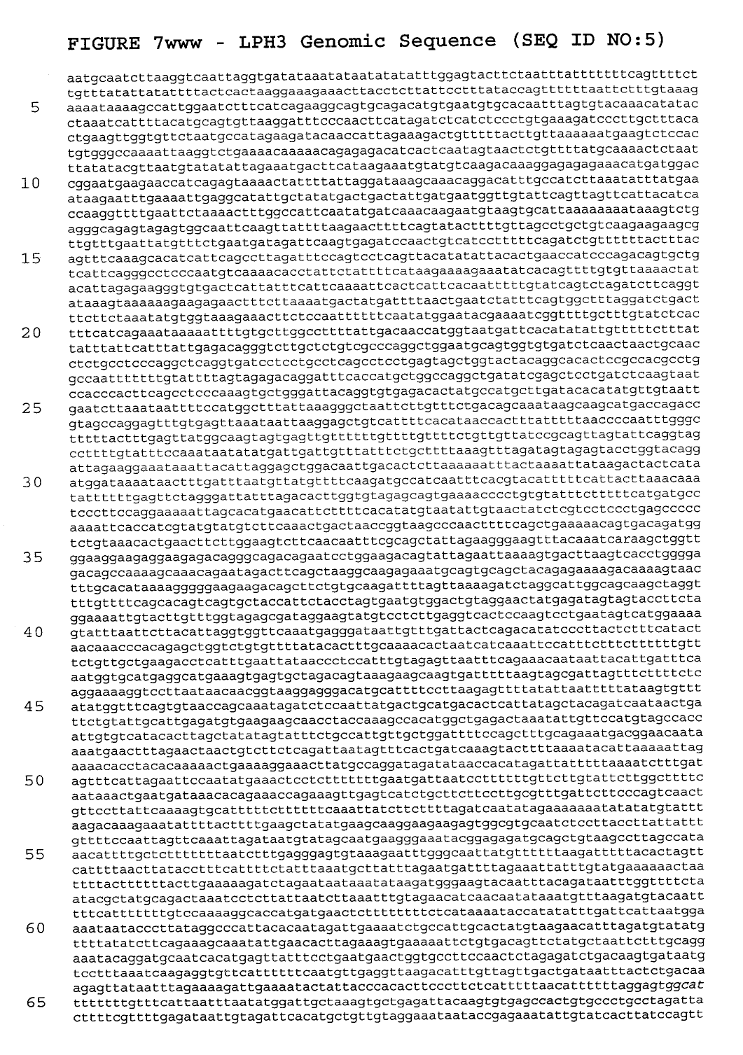

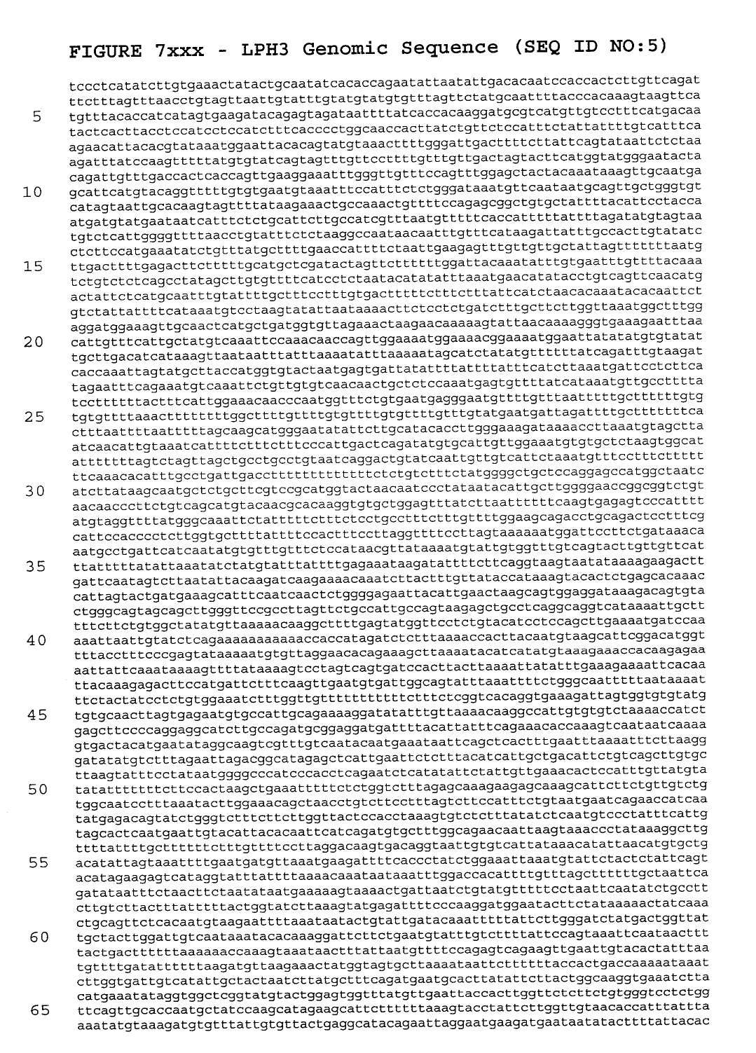

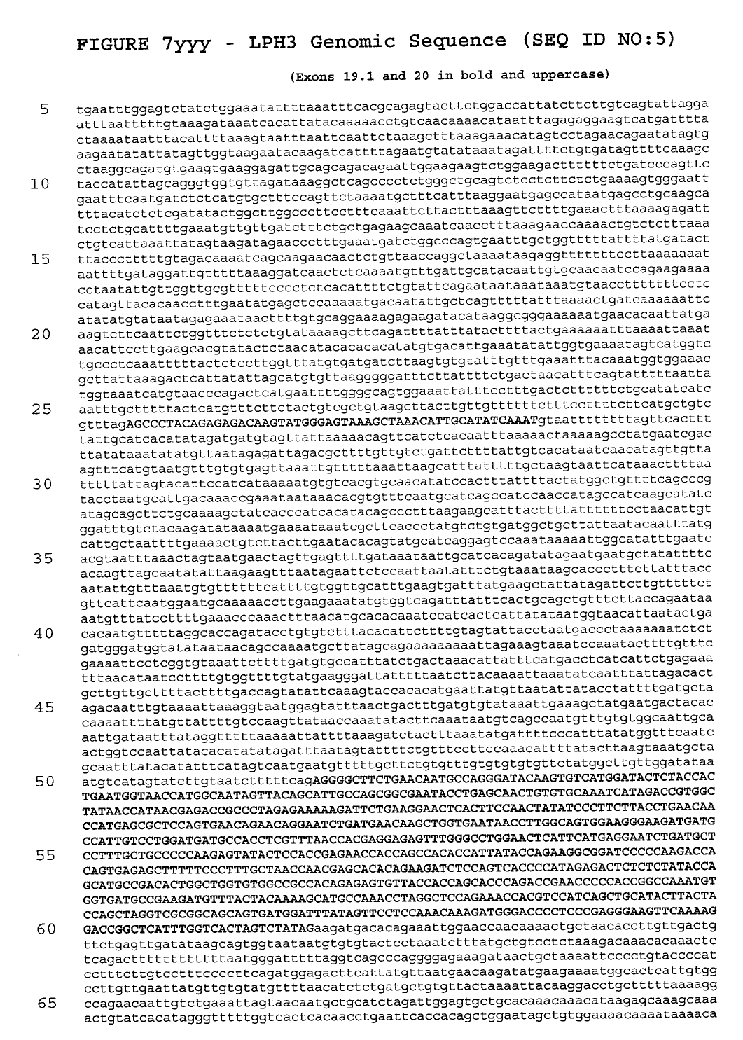

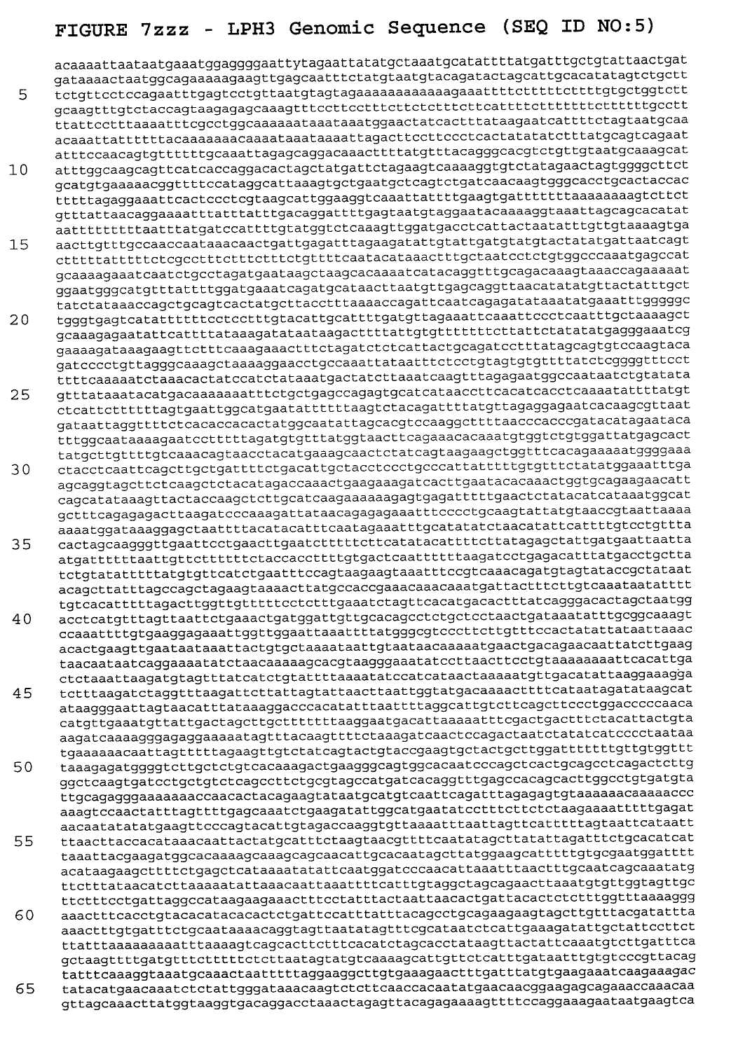

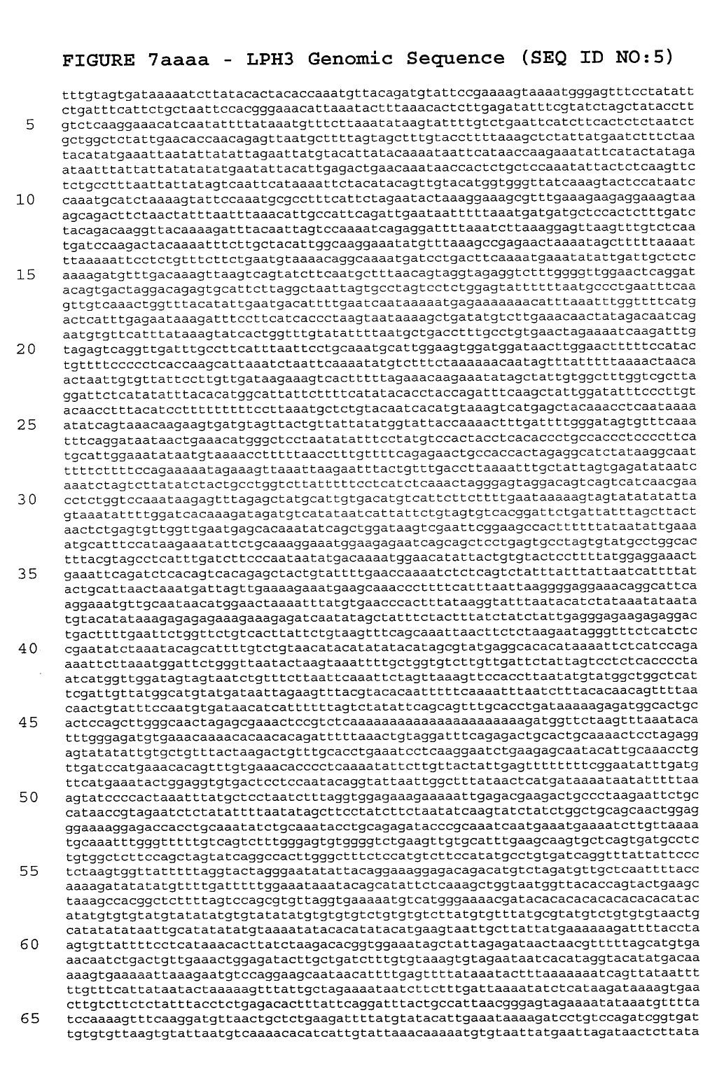

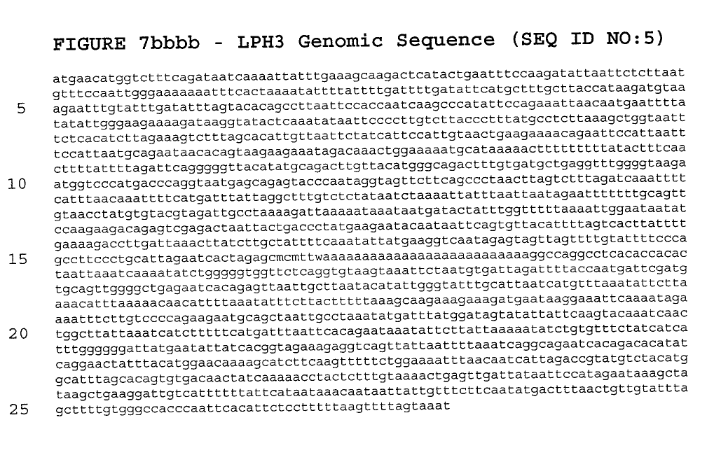

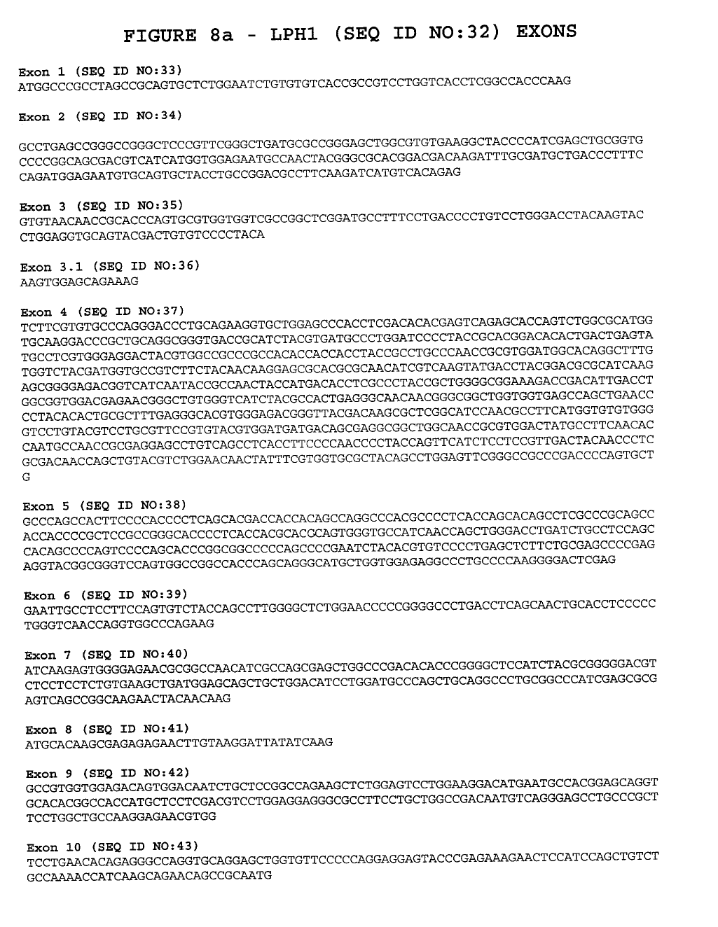

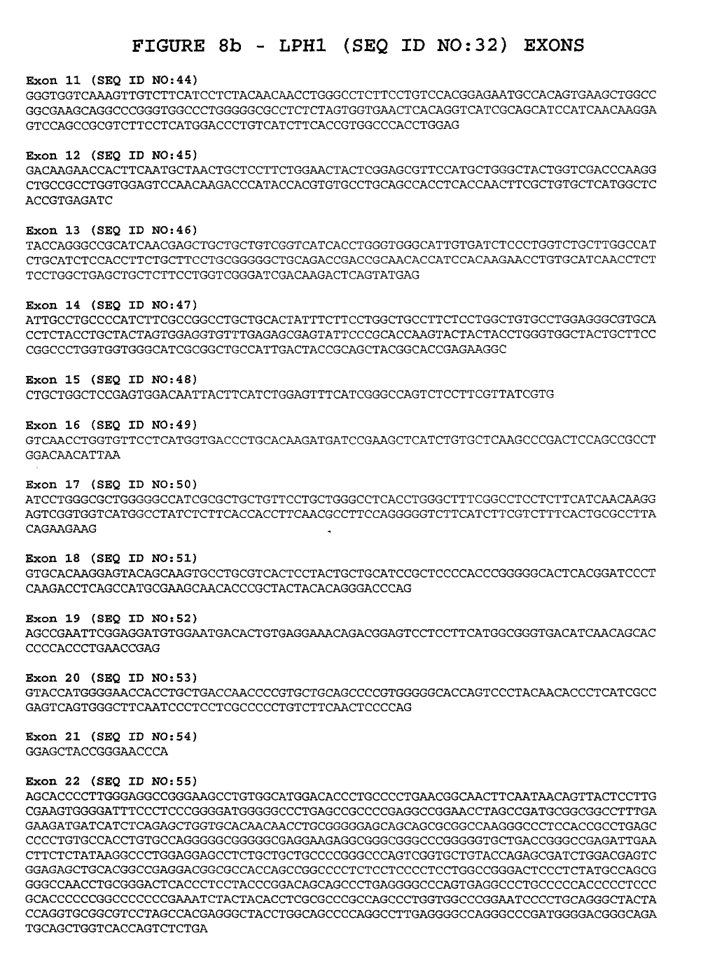

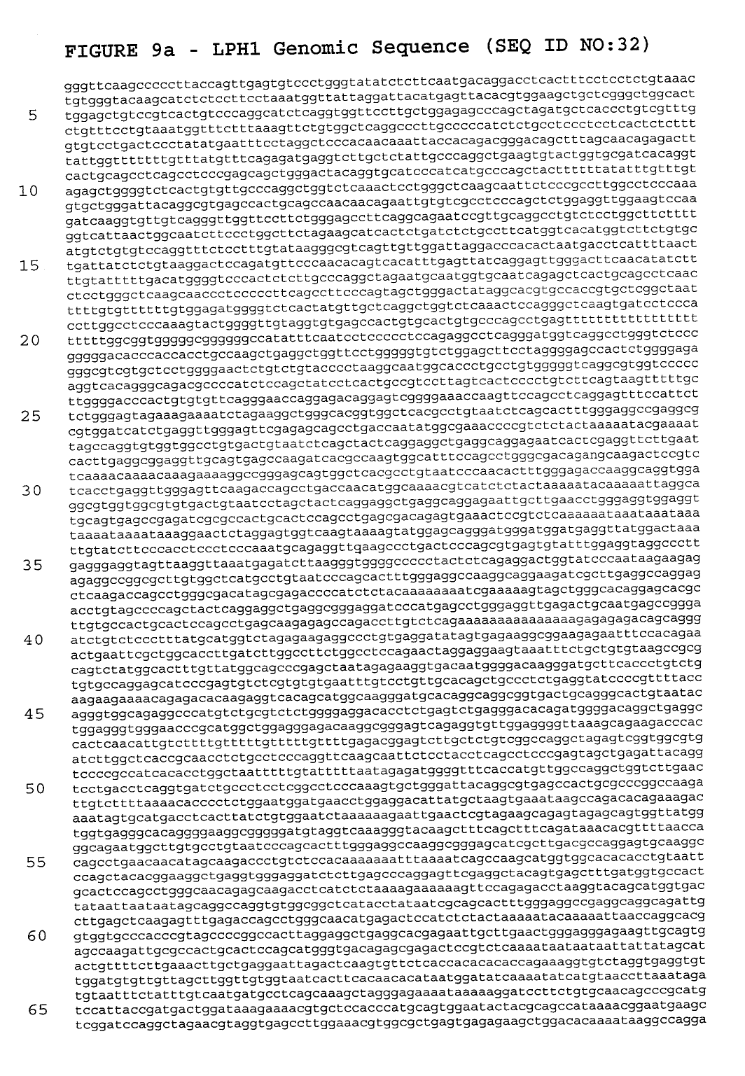

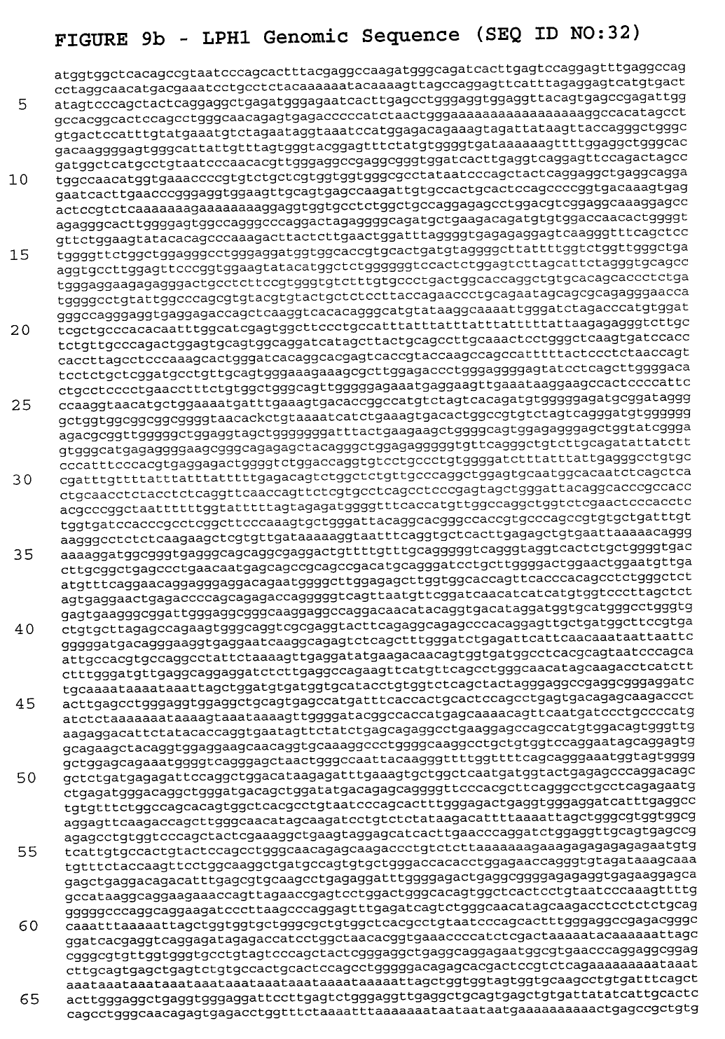

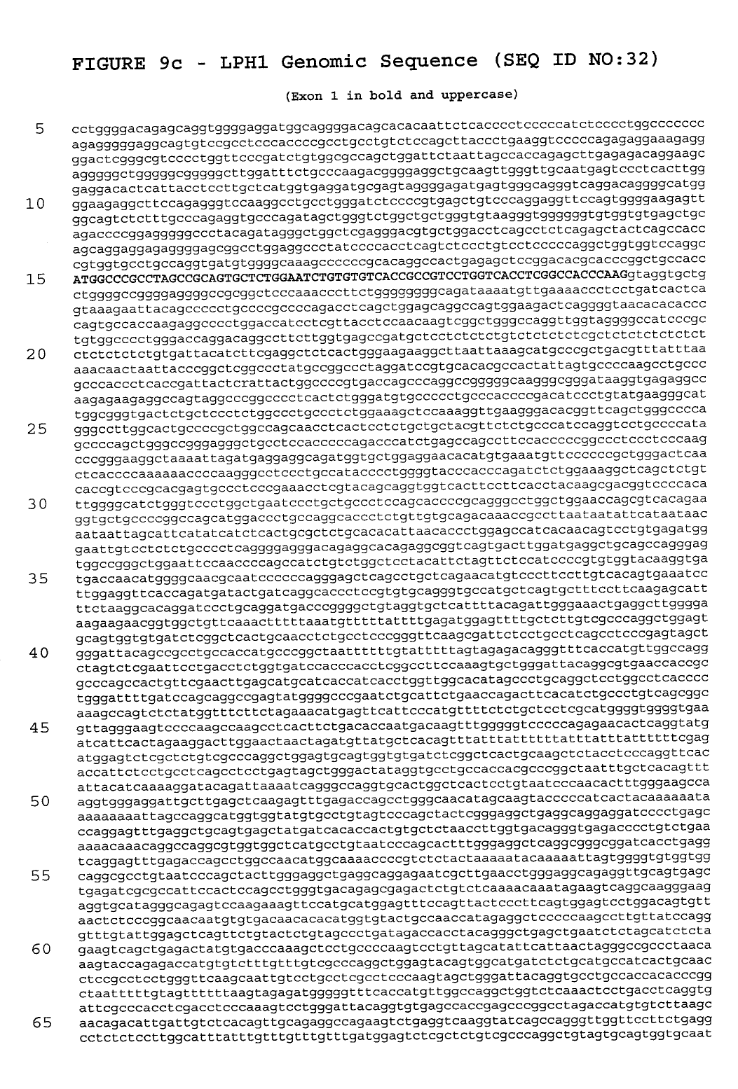

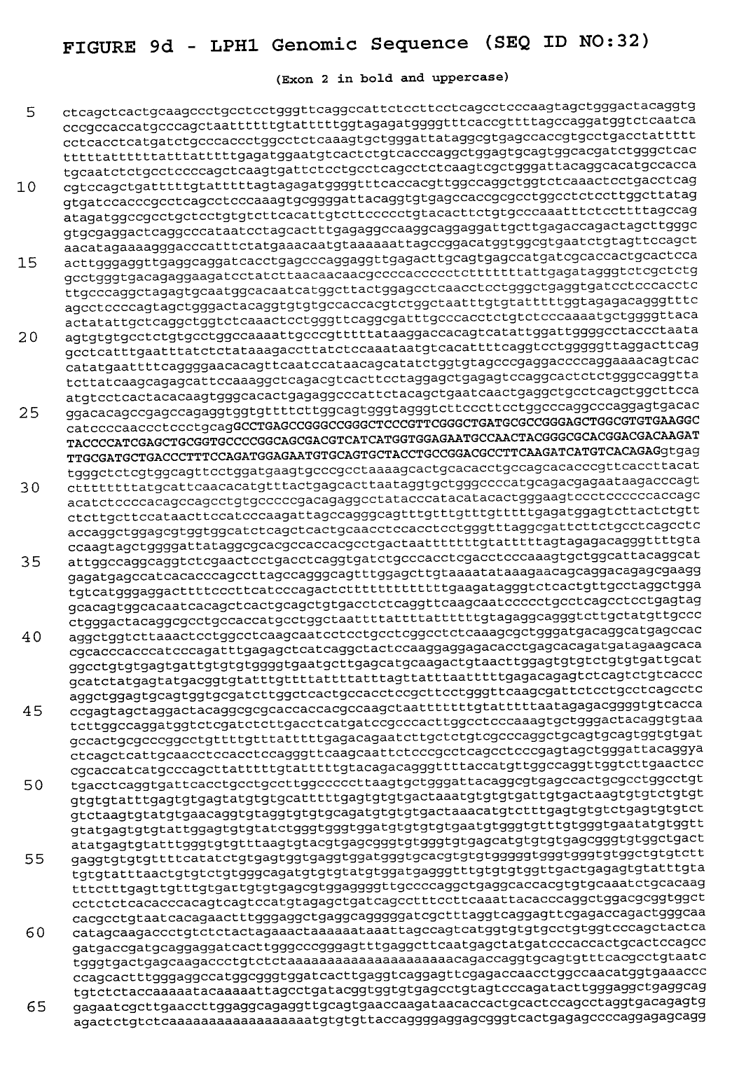

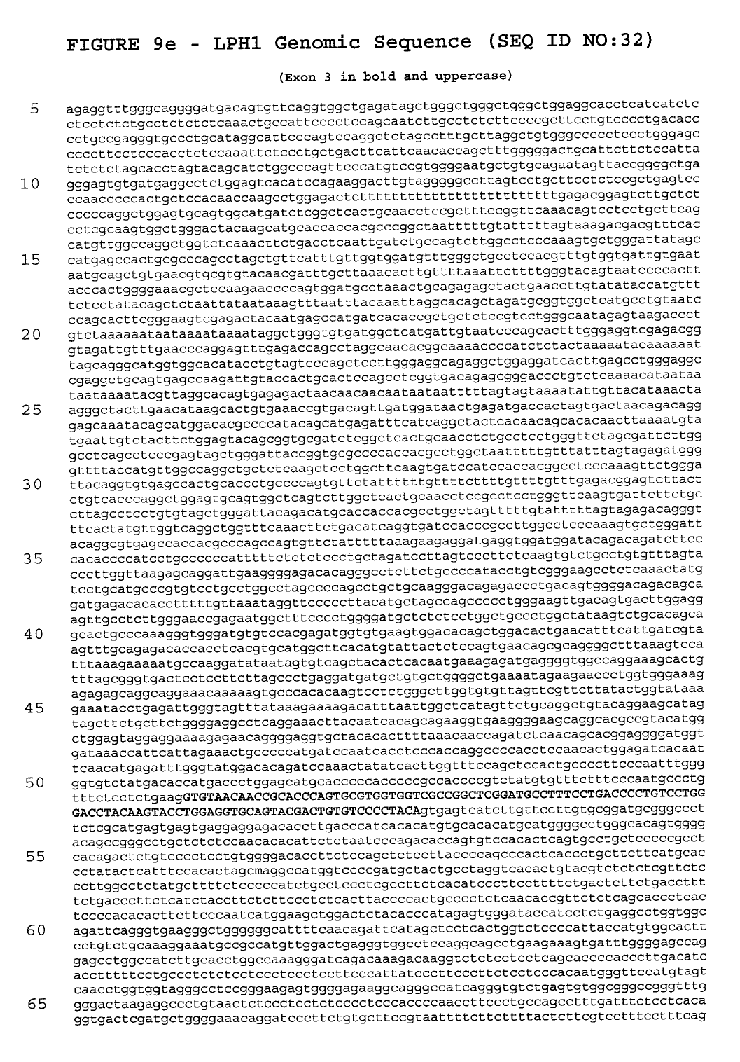

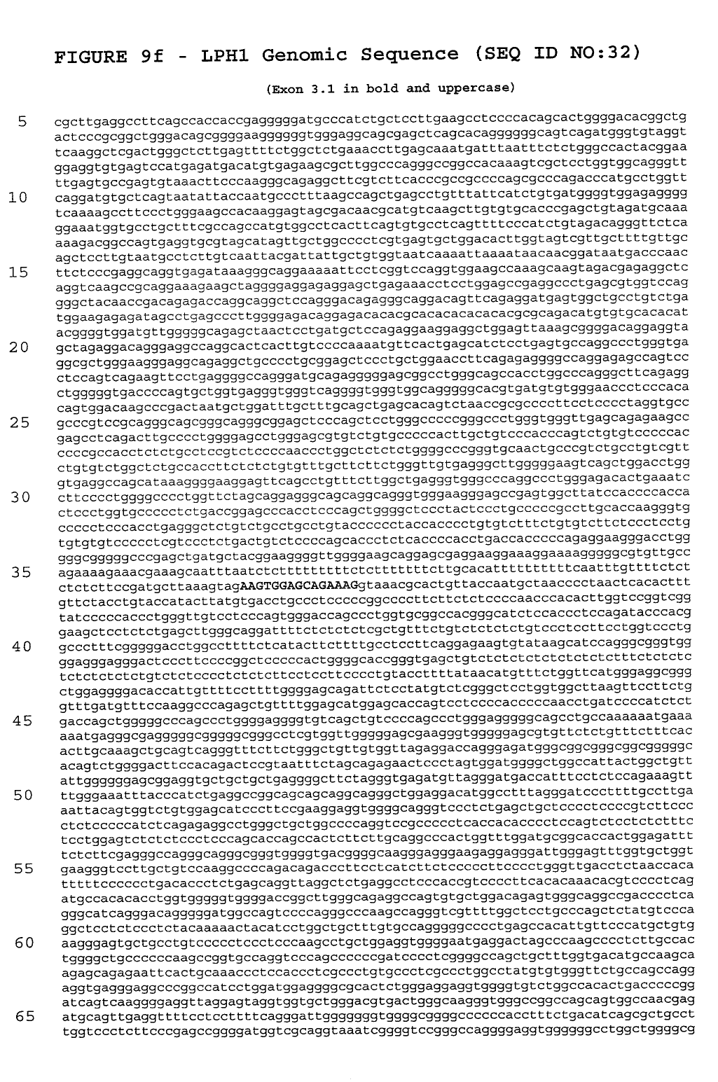

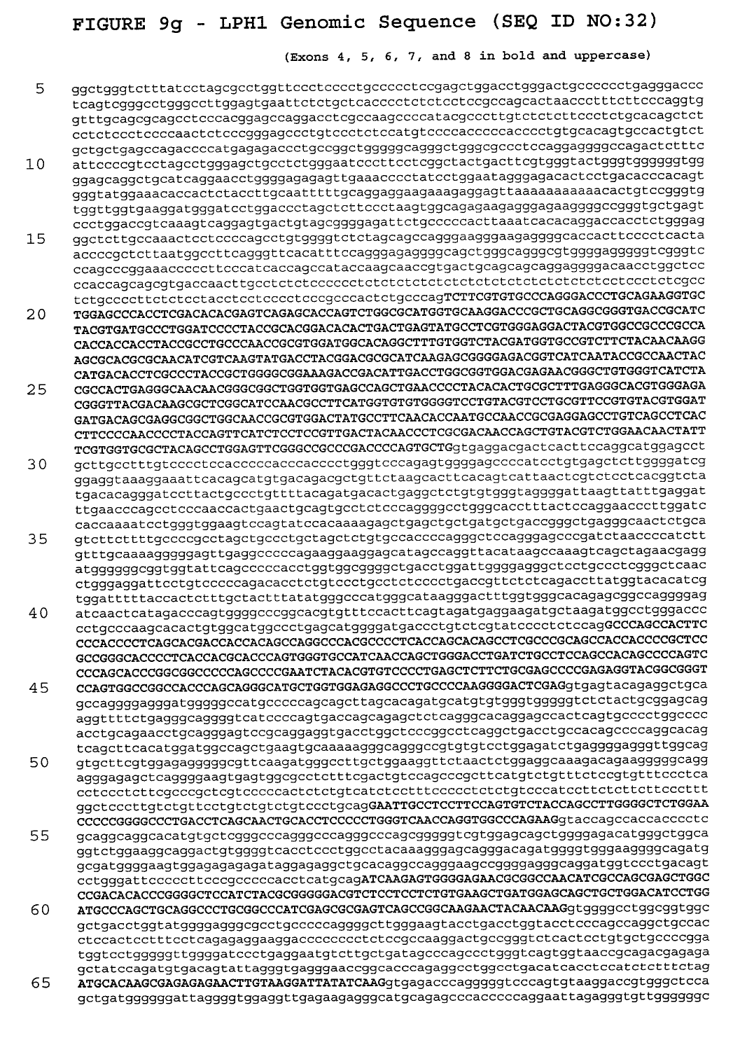

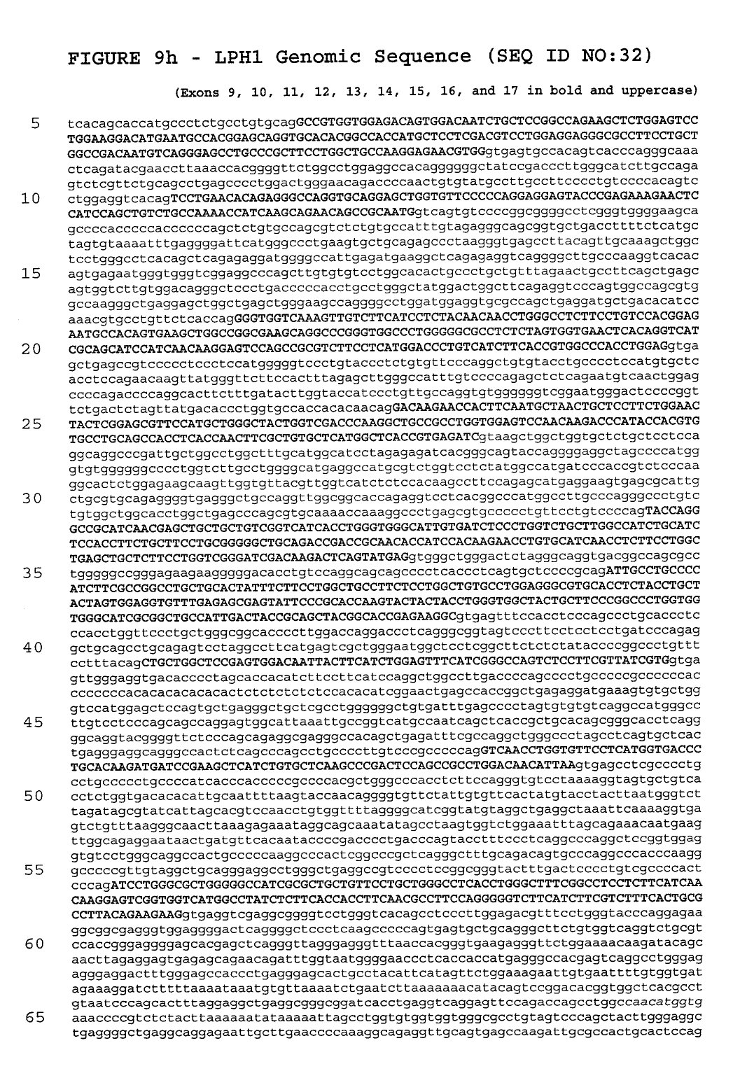

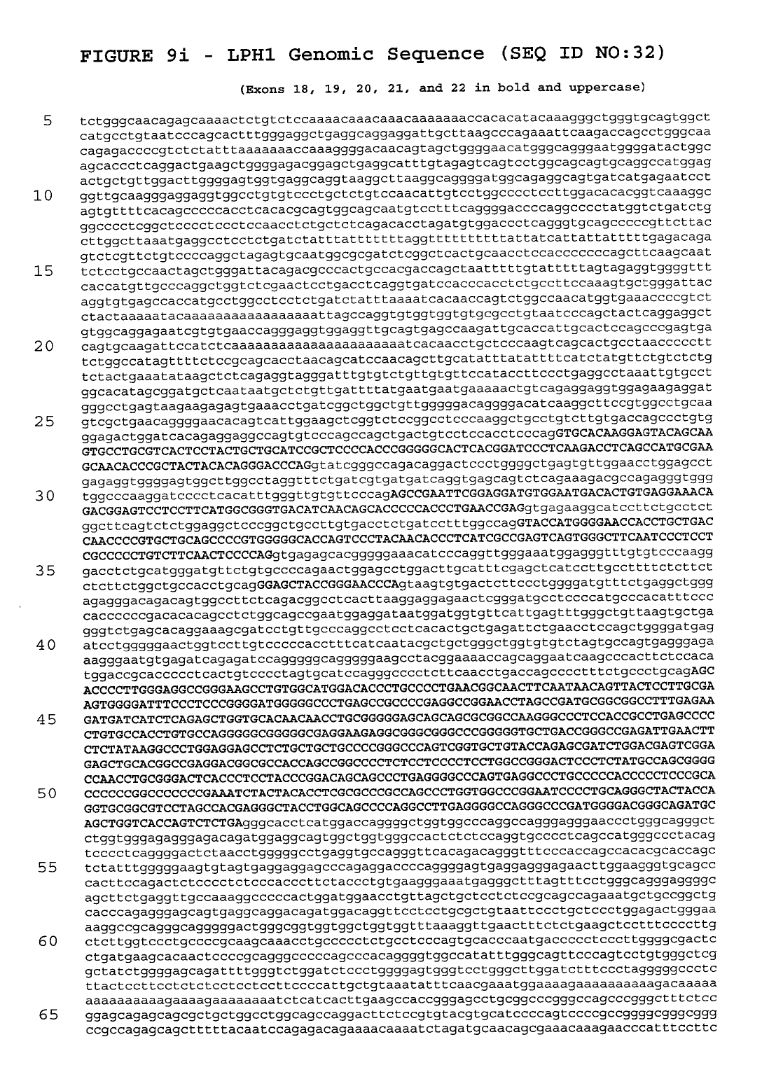

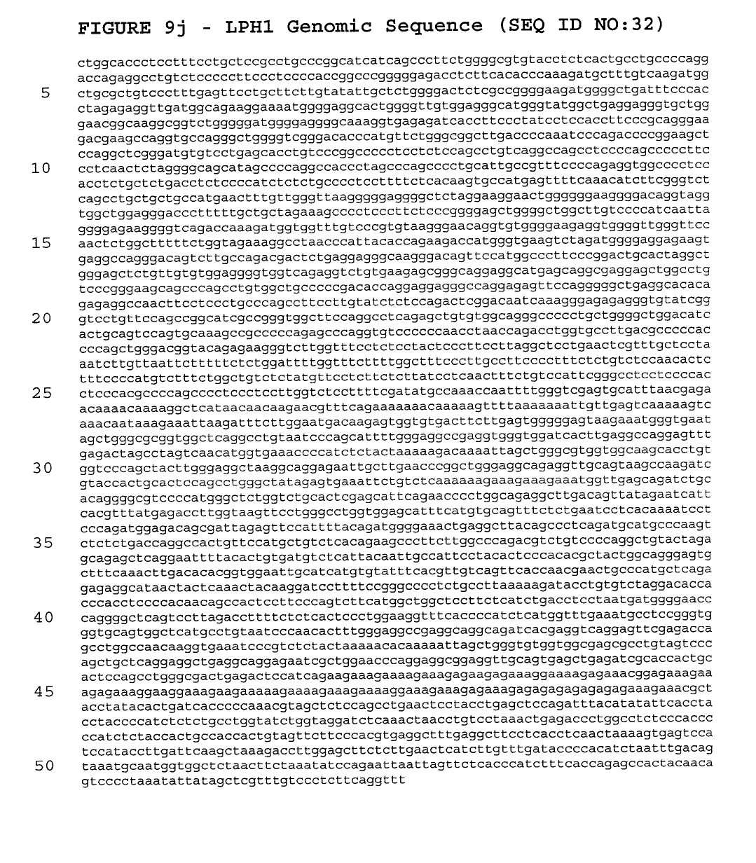

In other embodiments, the LPH3 and LPH1 nucleic acids of the present invention comprise at least a portion of the human genomic sequence of LPH3 as shown in SEQ ID NO:5 (See FIG. 7), or at least a portion of the human genomic sequence of human LPH1 as shown in SEQ ID NO:32 (See FIG. 9). In some embodiments, the nucleic acids of the present invention comprise an intronic portion of SEQ ID NO:5 or SEQ ID NO:32. In preferred embodiments, this intronic portion is at least 15, at least 30, at least 50, at least 100, or at least 200 consecutive bases from SEQ ID NO:5 or 32.

In still other embodiments of the present invention, the nucleotide sequences of the present invention may be engineered in order to alter an LPH3 coding sequence for a variety of reasons, including but not limited to, alterations which modify the cloning, processing and/or expression of the gene product. For example, mutations may be introduced using techniques that are well known in the art (e.g., site-directed mutagenesis to insert new restriction sites, to alter glycosylation patterns, to change codon preference, etc.).

In other embodiments of the present invention, variants of the disclosed LPH3 and LPH1 sequences are provided. In preferred embodiments, variants result from mutation, (i.e., a change in the nucleic acid sequence) and generally produce altered mRNAs or polypeptides whose structure or function may or may not be altered. Any given gene may have none, one, or many variant forms. Common mutational changes that give rise to variants are generally ascribed to deletions, additions or substitutions of nucleic acids. Each of these types of changes may occur alone, or in combination with the others, and at the rate of one or more times in a given sequence.

In some embodiments, the LPH3 polynucleotides encode peptides that bind the TIGR peptide. In this regard, it is contemplated that it is possible to modify the structure of a peptide for such purposes as increasing binding affinity of the LPH3 peptide for TIGR. (e.g. by employing directed evolution methods). A modified peptide can be produced in which the nucleotide sequence encoding the polypeptide has been altered, such as by substitution, deletion, or addition.

Moreover, as described above, variant forms of LPH3 and LPH1 are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of LPH3 and LPH1 disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur -containing (cysteine and methionine) (e.g., Stryer ed., Biochemistry, pg. 17-21, 2nd ed, WH Freeman and Co., 1981). Whether a change in the amino acid sequence of a peptide results in a functional homolog can be readily determined by assessing the ability of the variant peptide to function in a fashion similar to the wild-type protein. Peptides having more than one replacement can readily be tested in the same manner. Furthermore, even if a nulciec acid change results in a functional change, the altered polypeptide will, in some cases, also be of use in the present invention (e.g., where altered binding affinity is useful is useful in treating disease, or detecting LPH polypeptides, etc.).

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

As described in more detail below, variants may be produced by methods such as directed evolution or other techniques for producing combinatorial libraries of variants. In still other embodiments of the present invention, the nucleotide sequences of the present invention may be engineered in order to alter a LPH3 or LPH1 coding sequence including, but not limited to, alterations that modify the cloning, processing, localization, secretion, and/or expression of the gene product.

B. LPH3 Polypeptides

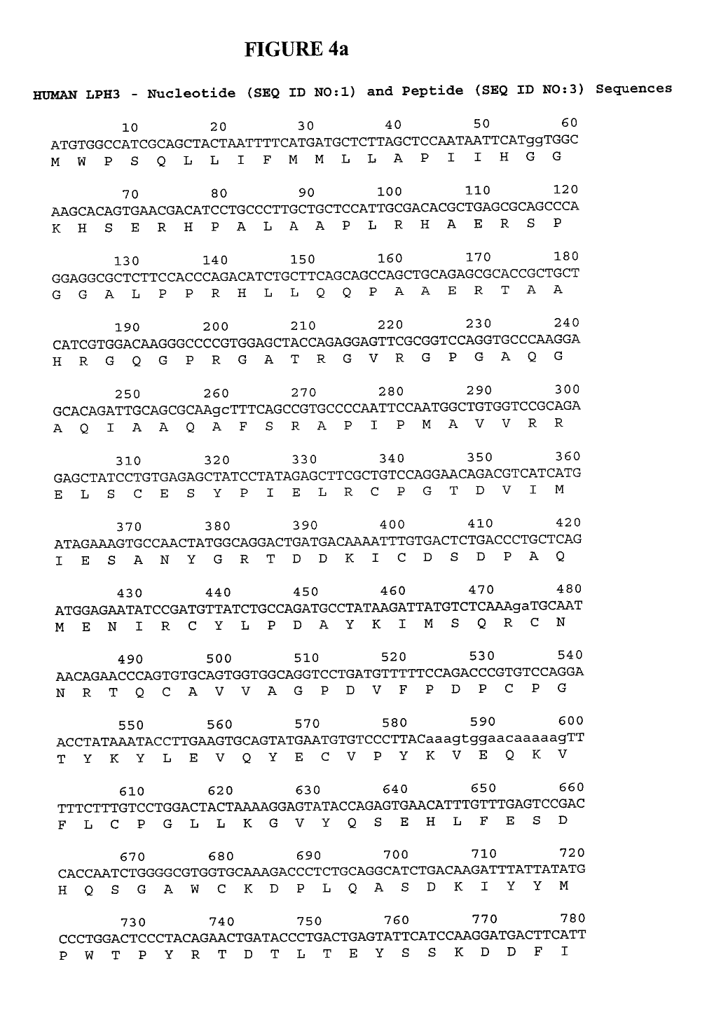

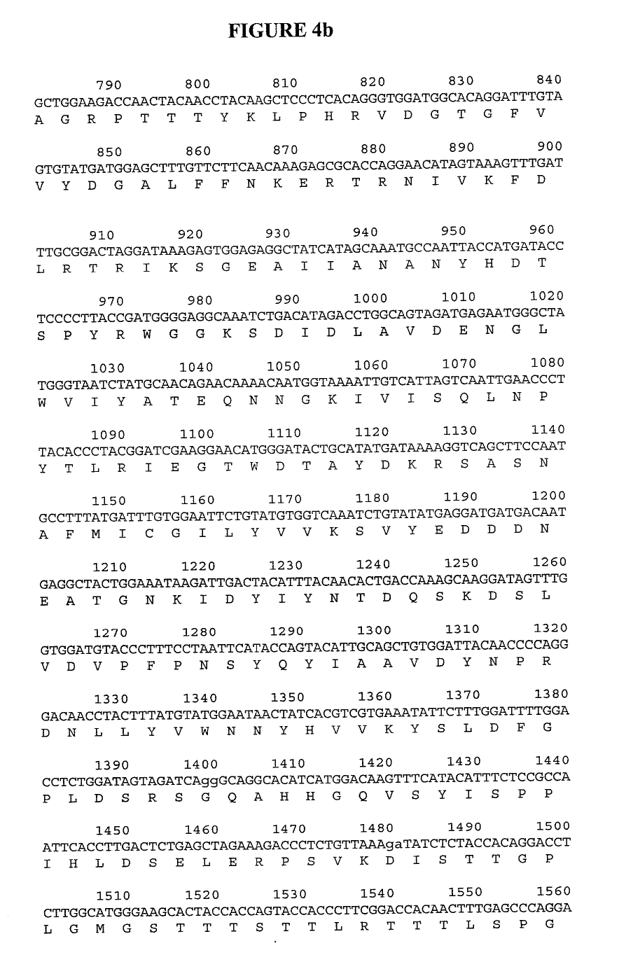

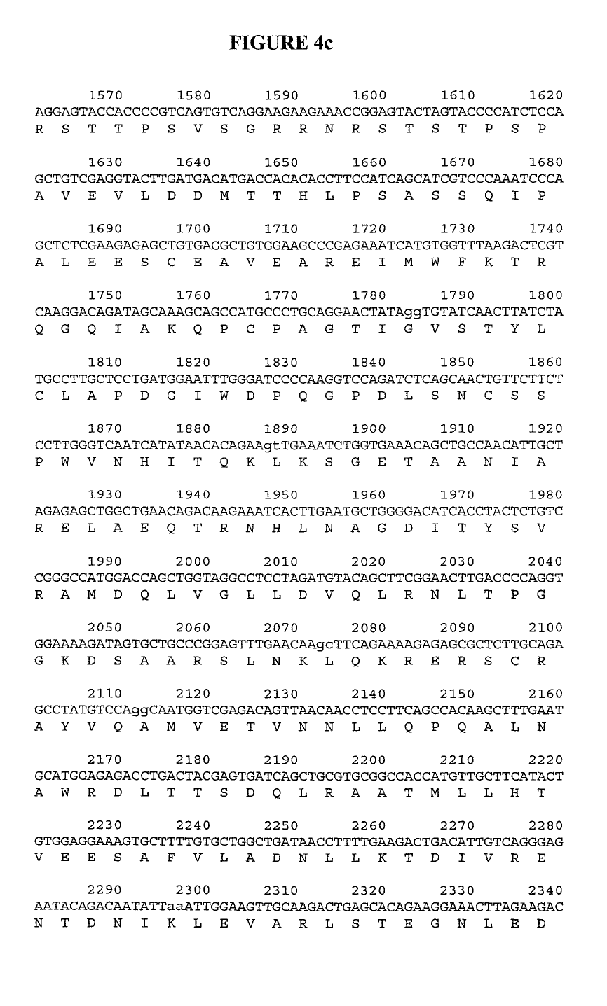

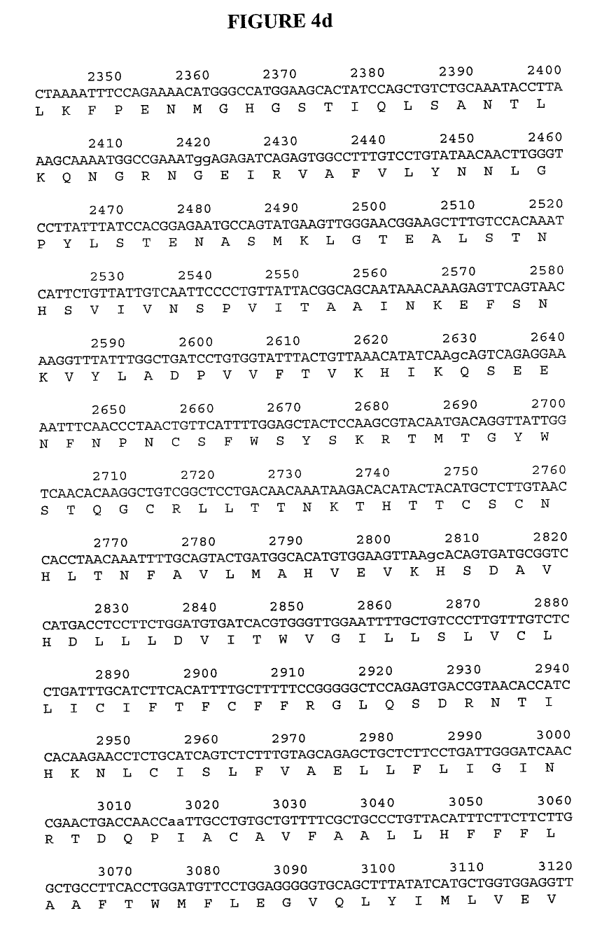

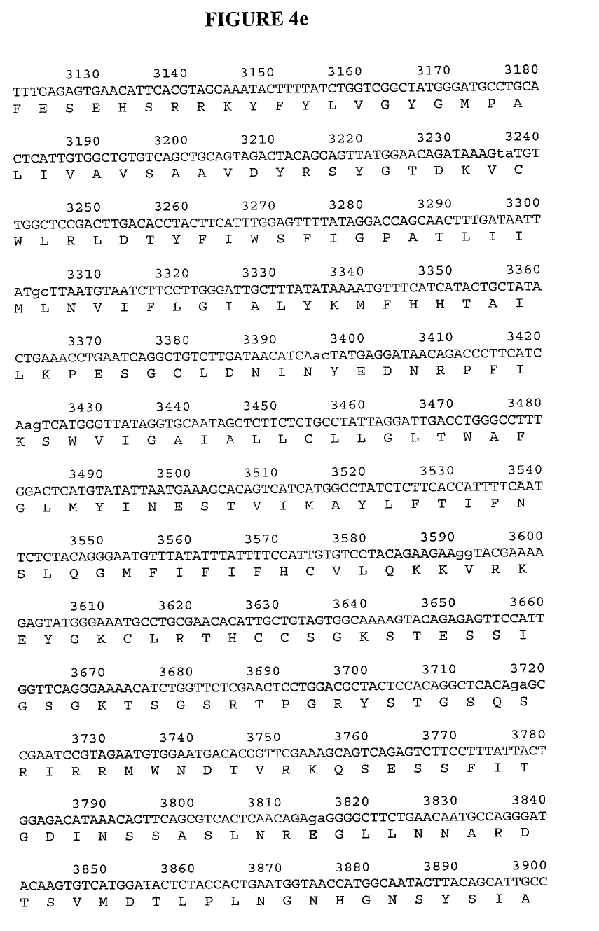

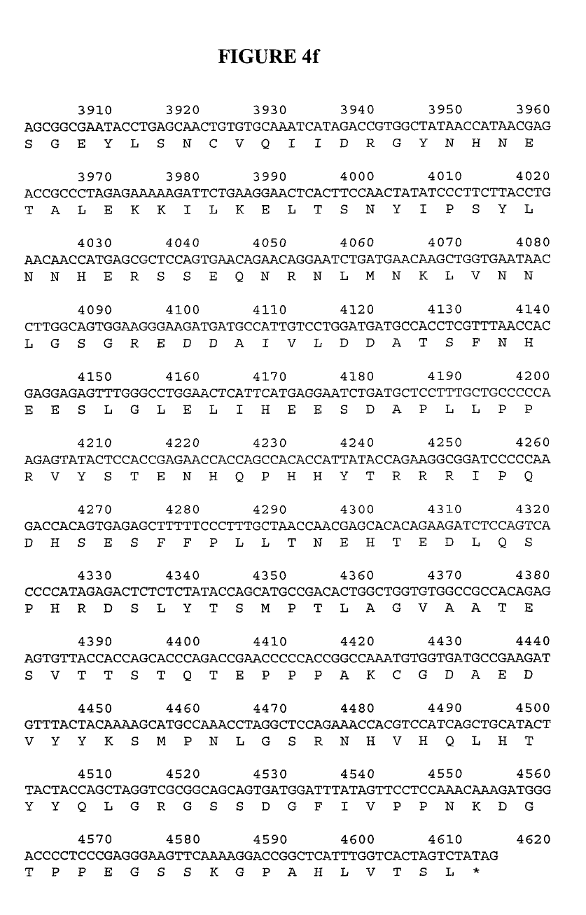

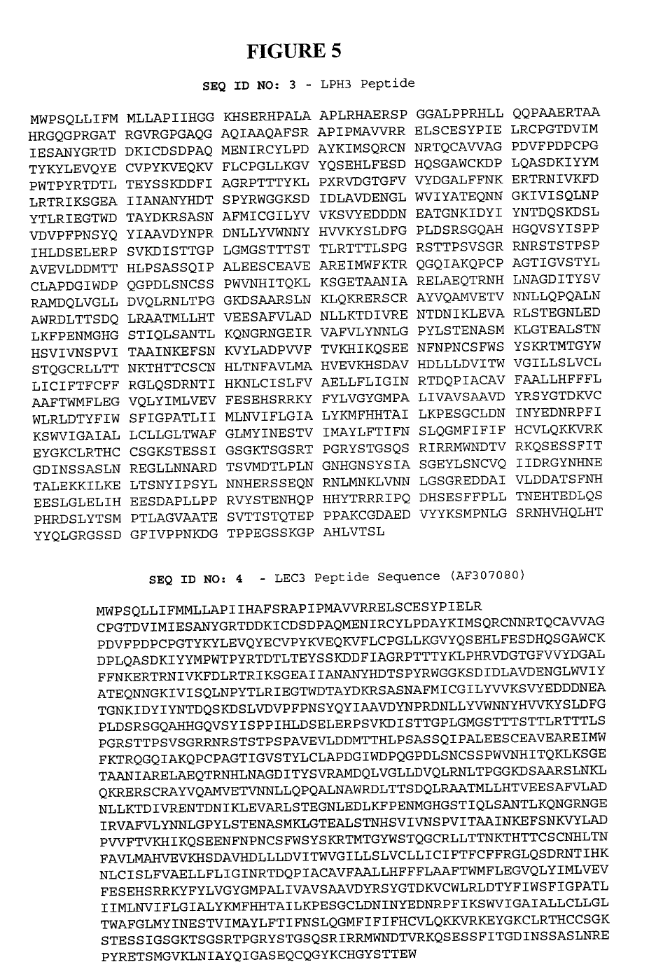

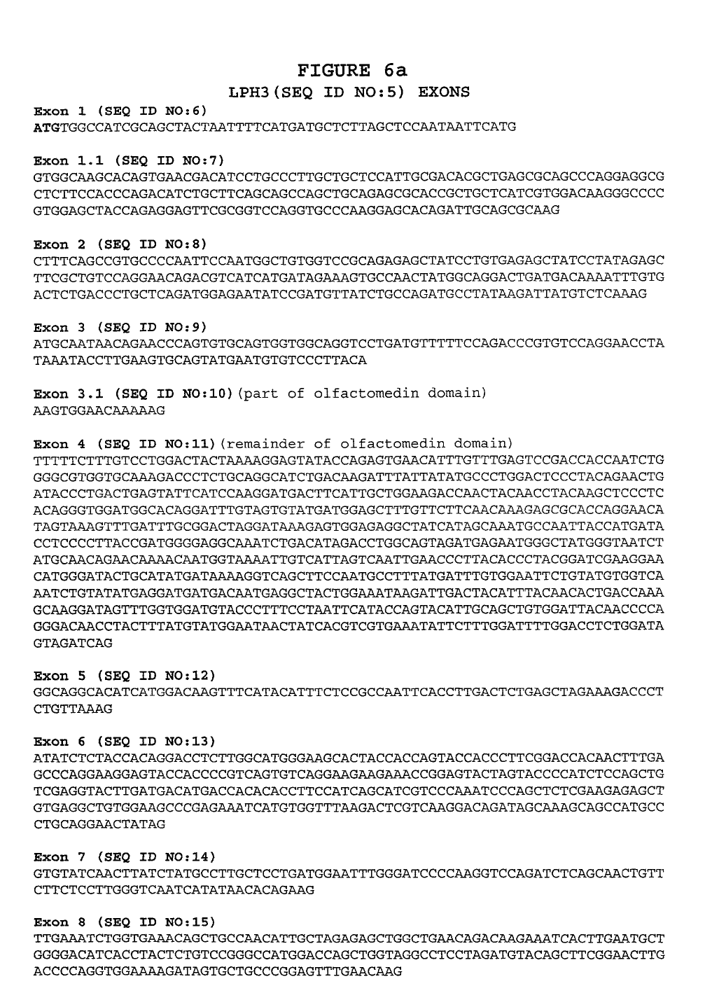

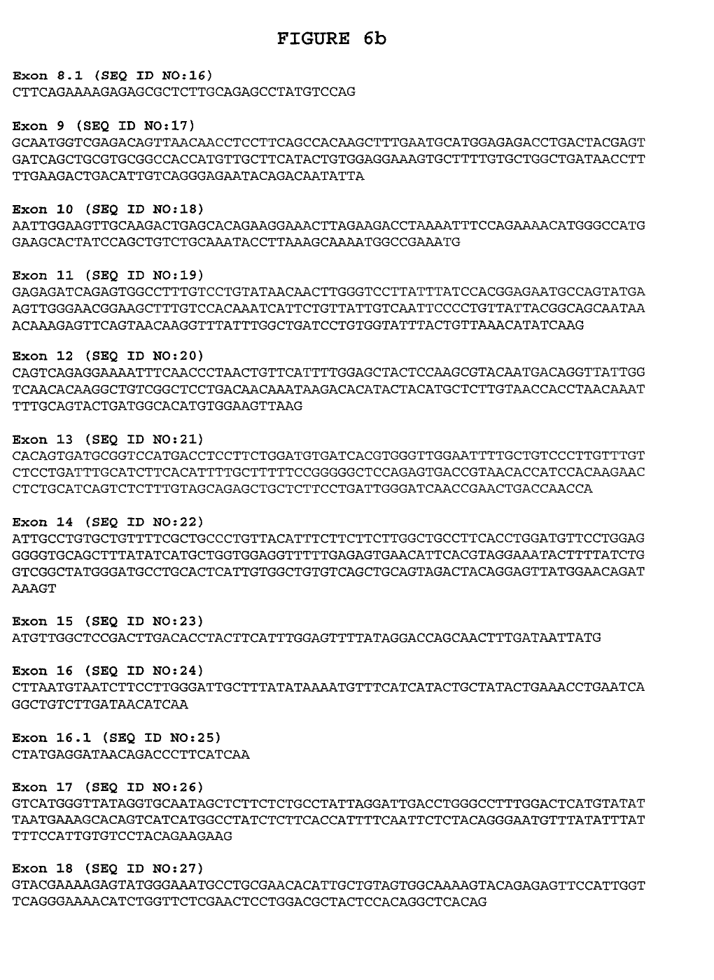

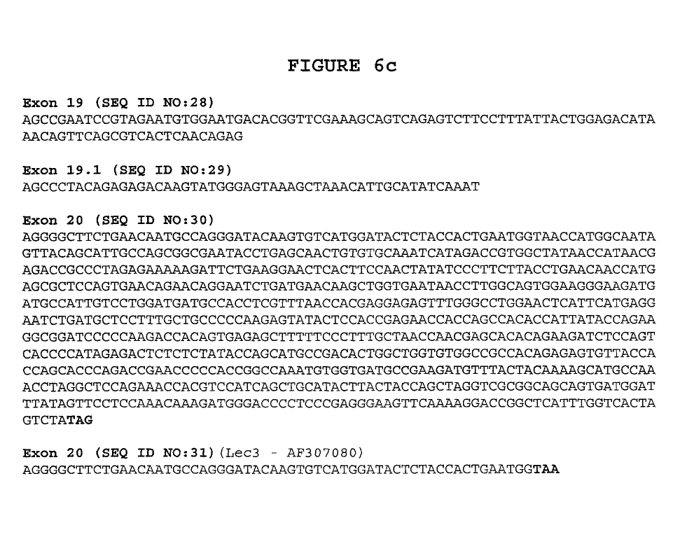

The present invention provides novel human LPH3 polypeptide sequences. Human LPH3 polypeptides (e.g., SEQ ID NO:3) are described in FIGS. 4 and 5. Other embodiments of the present invention provide fragments, fusion proteins or functional equivalents of LPH3 proteins. In still other embodiment of the present invention, nucleic acid sequences corresponding to these various LPH3 homologs and mutants may be used to generate recombinant DNA molecules that direct the expression of the LPH3 homologs and mutants in appropriate host cells. In some embodiments of the present invention, the LPH3 polypeptide may be a naturally purified product, in other embodiments it may be a product of chemical synthetic procedures, and in still other embodiments it may be produced by recombinant techniques using a prokaryotic or eukaryotic host (e.g., by bacterial, yeast, higher plant, insect and mammalian cells in culture). In some embodiments, depending upon the host employed in a recombinant production procedure, the polypeptide of the present invention may be glycosylated or may be non-glycosylated. In other embodiments, the polypeptides of the invention may also include an initial methionine amino acid residue. In preferred embodiments, the LPH3 peptides comprise an olfactomedin domain.

In one embodiment of the present invention, due to the inherent degeneracy of the genetic code, DNA sequences other than the polynucleotide sequences of SEQ ID NO:1 which encode substantially the same or a functionally equivalent amino acid sequence, may be used to clone and express human LPH3 peptides. In general, such polynucleotide sequences hybridize to SEQ ID NO:1 under conditions of high to medium stringency as described above. As will be understood by those of skill in the art, it may be advantageous to produce LPH3-encoding nucleotide sequences possessing non-naturally occurring codons. Therefore, in some preferred embodiments, codons preferred by a particular prokaryotic or eukaryotic host (Murray et al., Nucl. Acids Res., 17 [1989]) are selected, for example, to increase the rate of LPH3 peptide expression or to produce recombinant RNA transcripts having desirable properties, such as a longer half-life, than transcripts produced from naturally occurring sequences.

1. Vectors for Production of LPH3

The polynucleotides of the present invention may be employed for producing polypeptides by recombinant techniques. Thus, for example, the polynucleotide may be included in any one of a variety of expression vectors for expressing an LPH3 polypeptide. In some embodiments of the present invention, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments of the present invention provide recombinant constructs comprising one or more of the sequences as broadly described above (e.g., SEQ ID NOS:1 and 2). In some embodiments of the present invention, the constructs comprise a vector, such as a plasmid or viral vector, into which a sequence of the invention has been inserted, in a forward or reverse orientation. In still other embodiments, the heterologous structural sequence (e.g., SEQ ID NO:1) is assembled in appropriate phase with translation initiation and termination sequences. In preferred embodiments of the present invention, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); and 2) Eukaryotic—pWLNEO, pSV2CAT, pOG44, PXT1, pSG (Stratagene) pSVK3, pBPV, pMSG, pSVL (Pharmacia). Any other plasmid or vector may be used as long as they are replicable and viable in the host. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the DNA sequence in the expression vector is operatively linked to an appropriate expression control sequence(s) (promoter) to direct MRNA synthesis. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments of the present invention, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin bp 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

2. Host Cells for Production of LPH3

In a further embodiment, the present invention provides host cells containing the above-described constructs. In some embodiments of the present invention, the host cell is a higher eukaryotic cell (e.g., a mammalian or insect cell). In other embodiments of the present invention, the host cell is a lower eukaryotic cell (e.g., a yeast cell). In still other embodiments of the present invention, the host cell can be a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman, Cell 23:175 [1981]), C127, 3T3, 293, 293T, HeLa and BHK cell lines.

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation (See e.g., Davis et al., Basic Methods in Molecular Biology, [1986]). Alternatively, in some embodiments of the present invention, the polypeptides of the invention can be synthetically produced by conventional peptide synthesizers.

Proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989).

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

3. Purification of LPH3 Peptides

The present invention also provides methods for recovering and purifying LPH3 peptides from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In other embodiments of the present invention, protein refolding steps can be used as necessary, in completing configuration of the mature protein. In still other embodiments of the present invention, high performance liquid chromatography (HPLC) can be employed for final purification steps.

The present invention further provides polynucleotides having the coding sequence (e.g., SEQ ID NOs:1 and 2) fused in frame to a marker sequence which allows for purification of the polypeptide of the present invention. A non-limiting example of a marker sequence is a hexahistidine tag which may be supplied by a vector, preferably a pQE-9 vector, which provides for purification of the polypeptide fused to the marker in the case of a bacterial host, or, for example, the marker sequence may be a hemagglutinin (HA) tag when a mammalian host (e.g., COS-7 cells) is used. The HA tag corresponds to an epitope derived from the influenza hemagglutinin protein (Wilson et al., Cell, 37:767 [1984]).

4. Truncation Mutants of LPH3

In addition, the present invention provides fragments of LPH3. In some embodiments of the present invention, when expression of a portion of the LPH3 protein is desired, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al, J. Bacteriol., 169:751-757 [1987]) and Salmonella typhimurium and its in vitro activity has been demonstrated on recombinant proteins (Miller et al., Proc. Natl. Acad. Sci. USA 84:2718-1722 [1990]). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP.

5. Fusion Proteins Containing LPH3

The present invention also provides fusion proteins incorporating all or part of LPH3 (SEQ ID NO:3). Accordingly, in some embodiments of the present invention, the coding sequences (e.g. one or more of exons 1-20, shown in SEQ ID NOS 6-30) for the polypeptide can be incorporated as a part of a fusion gene including a nucleotide sequence encoding a different polypeptide. It is contemplated, for example, that this type of expression system will find use under conditions where it is desirable to produce, for example, an immunogenic fragment of an LPH3 protein. In some embodiments of the present invention, the VP6 capsid protein of rotavirus is used as an immunologic carrier protein for portions of the LPH3 polypeptide, either in the monomeric form or in the form of a viral particle. In other embodiments of the present invention, the nucleic acid sequences corresponding to the portion of LPH3 against which antibodies are to be raised can be incorporated into a fusion gene construct which includes coding sequences for a late vaccinia virus structural protein to produce a set of recombinant viruses expressing fusion proteins comprising a portion of LPH3 as part of the virion. It has been demonstrated with the use of immunogenic fusion proteins utilizing the hepatitis B surface antigen fusion proteins that recombinant hepatitis B virions can be utilized in this role as well. Similarly, in other embodiments of the present invention, chimeric constructs coding for fusion proteins containing a portion of LPH3 and the poliovirus capsid protein are created to enhance immunogenicity of the set of polypeptide antigens.

In still other embodiments of the present invention, the multiple antigen peptide system for peptide-based immunization can be utilized. In this system, a desired portion of LPH3 (e.g. a portion expressing the olfactomedin domain of LPH3 as encoded by SEQ ID NOS:10 and 11) is obtained directly from organo-chemical synthesis of the peptide onto an oligomeric branching lysine core. In other embodiments of the present invention, antigenic determinants of the LPH3 proteins can also be expressed and presented by bacterial cells.

In addition to utilizing fusion proteins to enhance immunogenicity, it is widely appreciated that fusion proteins can also facilitate the expression of proteins, such as the LPH3 protein of the present invention. Accordingly, in some embodiments of the present invention, LPH3 peptides can be generated as a glutathione-S-transferase (i.e., GST fusion protein). It is contemplated that such GST fusion proteins will enable easy purification of LPH3 peptides, such as by the use of glutathione-derivatized matrices (See e.g, Ausabel et al. (eds.), Current Protocols in Molecular Biology, John Wiley & Sons, NY [1991]). In another embodiment of the present invention, a fusion gene coding for a purification leader sequence, such as a poly-(His)/enterokinase cleavage site sequence at the N-terminus of the desired portion of LPH3, can allow purification of the expressed LPH3 fusion protein by affinity chromatography using a Ni²⁺ metal resin. In still another embodiment of the present invention, the purification leader sequence can then be subsequently removed by treatment with enterokinase.

Techniques for making fusion genes are well known. Essentially, the joining of various DNA fragments coding for different polypeptide sequences is performed in accordance with conventional techniques, for example, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment of the present invention, the fusion gene can be synthesized by conventional techniques including automated DNA synthesizers. Alternatively, in other embodiments of the present invention, PCR amplification of gene fragments can be carried out using anchor primers which give rise to complementary overhangs between two consecutive gene fragments which can subsequently be annealed to generate a chimeric gene sequence (See e.g., Current Protocols in Molecular Biology, supra).

6. Variants of LPH3

Still other embodiments of the present invention provide mutant or variant forms of LPH3 (i.e., muteins). It is possible to modify the structure of LPH3 peptide for such purposes as enhancing therapeutic or prophylactic efficacy, or stability (e.g., ex vivo shelf life, and/or resistance to proteolytic degradation in vivo). A modified peptide can be produced in which the amino acid sequence has been altered, such as by amino acid substitution, deletion, or addition. In some embodiments, variants of LPH3 may be produced with have a greater affinity for the TIGR peptide as compared to the wild type LPH3 peptide shown in SEQ ID NO:3.

Moreover, as described above, variant forms (e.g., mutants) of the subject LPH3 proteins are also contemplated as being equivalent to those peptides and DNA molecules that are set forth in more detail. For example, as described above, the present invention encompasses mutant and variant proteins that contain conservative or non-conservative amino acid substitutions.

The present invention further contemplates a method of generating sets of combinatorial mutants of the present LPH3 proteins, as well as truncation mutants, and is especially useful for identifying potential variant sequences (i.e., homologs) that are functional in binding TIGR proteins. The purpose of screening such combinatorial libraries is to generate, for example, novel LPH3 homologs which can act as either agonists or antagonists, or alternatively, possess novel activities all together.

Therefore, in some embodiments of the present invention, LPH3 homologs are engineered by the present method to provide greater binding affinity for the TIGR peptide. Such proteins, may, for example, be used in gene therapy protocols (see below).

Still other embodiments of the present invention provide LPH3 homologs which have intracellular half-lives dramatically different than the corresponding wild-type protein. For example, the altered protein can be rendered either more stable or less stable to proteolytic degradation or other cellular process which result in destruction of, or otherwise inactivate LPH3. Such homologs, and the genes which encode them, can be utilized to alter the location of LPH3 expression by modulating the half-life of the protein. For instance, a short half-life can give rise to more transient LPH3 biological effects and, when part of an inducible expression system, can allow tighter control of LPH3 levels within the cell. As above, such proteins, and particularly their recombinant nucleic acid constructs, can be used, for example, in gene therapy protocols.

In still other embodiments of the present invention, LPH3 homologs are generated by the combinatorial approach to act as antagonists, in that they are able to interfere with the ability of the corresponding wild-type protein to regulate cell function (e.g. interfere with the ability of wild type human LPH3 from binding a ligand such as the TIGR protein).

In some embodiments of the combinatorial mutagenesis approach of the present invention, the amino acid sequences for a population of LPH3 homologs or other related proteins are aligned, preferably to promote the highest homology possible. Such a population of variants can include, for example, LPH3 homologs from one or more species, or LPH3 homologs from the same species but which differ due to mutation. Amino acids which appear at each position of the aligned sequences are selected to create a degenerate set of combinatorial sequences.

In a preferred embodiment of the present invention, the combinatorial LPH3 library is produced by way of a degenerate library of genes encoding a library of polypeptides which each include at least a portion of potential LPH3 protein sequences. For example, a mixture of synthetic oligonucleotides can be enzymatically ligated into gene sequences such that the degenerate set of potential LPH3 sequences are expressible as individual polypeptides, or alternatively, as a set of larger fusion proteins (e.g., for phage display) containing the set of LPH3 sequences therein.

There are many ways by which the library of potential LPH3 homologs can be generated from a degenerate oligonucleotide sequence. In some embodiments, chemical synthesis of a degenerate gene sequence is carried out in an automatic DNA synthesizer, and the synthetic genes are ligated into an appropriate gene for expression. The purpose of a degenerate set of genes is to provide, in one mixture, all of the sequences encoding the desired set of potential LPH3 sequences. The synthesis of degenerate oligonucleotides is well known in the art (See e.g., Narang, Tetrahedron Lett., 39:3 9 [1983]). Such techniques have been employed in the directed evolution of other proteins (See e.g., U.S. Pat. Nos. 5,223,409, 5,198,346, and 5,096,815, each of which is incorporated herein by reference).

It is contemplated that the LPH3 nucleic acids (e.g., SEQ ID NO:1, 2, and 5, and fragments and variants thereof) can be utilized as starting nucleic acids for directed evolution. These techniques can be utilized to develop LPH3 variants having desirable properties such as increased or decreased binding affinity for TIGR.

In some embodiments, artificial evolution is performed by random mutagenesis (e.g., by utilizing error-prone PCR to introduce random mutations into a given coding sequence). This method requires that the frequency of mutation be finely tuned. As a general rule, beneficial mutations are rare, while deleterious mutations are common. The ideal number of base substitutions for targeted gene is usually between 1.5 and 5 (Moore and Arnold, Nat. Biotech., 14, 458-67 [1996]). After mutagenesis, the resulting clones are selected for desirable activity. Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or sexual PCR procedures (e.g., U.S. Pat. Nos. 5,837,458; and 5,733,731; both of which are herein incorporated by reference). Gene shuffling involves random fragmentation of several mutant DNAs followed by their reassembly by PCR into full length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences.

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis or recombination of LPH3 homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

7. Chemical Synthesis of LPH3

In some embodiments of the invention, the coding sequence of LPH3 is synthesized, whole or in part, using chemical methods well known in the art (See e.g., Caruthers et al., Nucl. Acids Res. Symp. Ser., 7:215-233 [1980]). In other embodiments of the present invention, the protein itself is produced using chemical methods to synthesize either an entire LPH3 amino acid sequence or a portion thereof. For example, peptides can be synthesized by solid phase techniques, cleaved from the resin, and purified by preparative high performance liquid chromatography (See e.g., Creighton, Proteins Structures And Molecular Principles, W H Freeman and Co, New York N.Y. [1983]). In other embodiments of the present invention, the composition of the synthetic peptides is confirmed by amino acid analysis or sequencing (See e.g., Creighton, supra).

Direct peptide synthesis can be performed using various solid-phase techniques (Roberge et al., Science 269:202-204 [1995]) and automated synthesis may be achieved, for example, using ABI 431A Peptide Synthesizer (Perkin Elmer) in accordance with the instructions provided by the manufacturer. Additionally, the amino acid sequence of LPH3, or any part thereof, may be altered during direct synthesis and/or combined using chemical methods with other sequences to produce a variant polypeptide.

8. Generation of LPH3 Antibodies

Antibodies can be generated to allow for the detection of LPH3 proteins. The antibodies may be prepared using various immunogens. In one embodiment, the immunogen is a human LPH3 peptide (e.g., SEQ ID NO:3) to generate antibodies that recognize human LPH3. Such antibodies include, but are not limited to polyclonal, monoclonal, chimeric, single chain, Fab fragments, Fab expression libraries, and humanized antibodies.

Various procedures known in the art may be used for the production of polyclonal antibodies directed against LPH3. For the production of antibody, various host animals can be immunized by injection with the peptide corresponding to the LPH3 epitope including but not limited to rabbits, chickens, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies directed toward LPH3, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, NY). These include but are not limited to the hybridoma technique originally developed by Köhler and Milstein (Köhler and Milstein, Nature 256:495-497 [1975]), as well as the trioma technique, the human B-cell hybridoma technique (See e.g., Kozbor et al., Immunol. Tod., 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

In an additional embodiment of the invention, monoclonal antibodies are produced in germ-free animals utilizing technology such as that described in PCT/US90/02545. Furthermore, it is contemplated that human antibodies will be generated by human hybridomas (Cote et al., Proc. Natl. Acad. Sci. USA 80:2026-2030 [1983]) or by transforming human B cells with EBV virus in vitro (Cole et al., in Monoclonal Antibodies and Cancer Therapy, Alan R. Liss, pp. 77-96 [1985]).

In addition, it is contemplated that techniques described for the production of single chain antibodies (U.S. Pat. No. 4,946,778; herein incorporated by reference) will find use in producing LPH3 specific single chain antibodies. An additional embodiment of the invention utilizes the techniques described for the construction of Fab expression libraries (Huse et al., Science 246:1275-1281 [1989]) to allow rapid and easy identification of monoclonal Fab fragments with the desired specificity for LPH3.

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. As is well known in the art, the immunogenic peptide should be provided free of the carrier molecule used in any immunization protocol. For example, if the peptide was conjugated to KLH, it may be conjugated to BSA, or used directly, in a screening assay.

The foregoing antibodies can be used in methods known in the art relating to the localization and structure of LPH3 (e.g., for Western blotting), measuring levels thereof in appropriate biological samples, etc. The antibodies can be used to detect LPH3 in a biological sample from an individual (e.g. to determine if the individual is under or overexpressing LPH3). The biological sample can be a biological fluid, such as, but not limited to, blood, serum, plasma, interstitial fluid, urine, cerebrospinal fluid, and the like, containing cells.

The biological samples can then be tested directly for the presence of human LPH3 using an appropriate strategy (e.g., ELISA or radioimmunoassay) and format (e.g., microwells, and dipstick), etc. Alternatively, proteins in the sample can be size separated (e.g., by polyacrylamide gel electrophoresis (PAGE), in the presence or not of sodium dodecyl sulfate (SDS), and the presence of LPH3 detected by immunoblotting (Western blotting). Immunoblotting techniques are generally more effective with antibodies generated against a peptide corresponding to an epitope of a protein, and hence, are particularly suited to the present invention.

Another method uses antibodies as agents to alter signal transduction. Specific antibodies that bind to the binding domains of LPH3 or other proteins involved in intracellular signalling can be used to inhibit the interaction between the various proteins and their interaction with other ligands. For example, it is believed that LPH3 is a transmembrane protein that is capable of binding the TIGR peptide. Antibodies that bind to LPH3 (e.g. at the olfactomedin domain) can be used therapeutically to inhibit interactions of the protein complex in the signal transduction pathways.

9. Transgenic Animals Expressing Exongenous LPH3 Genes and Homologs, Mutants, and Variants Thereof.

The present invention contemplates the generation of transgenic animals comprising an exogenous LPH3 gene (e.g. SEQ ID NO:1, SEQ ID NO:2, or SEQ ID NO:5) or homologs, mutants, or variants thereof. In preferred embodiments, the transgenic animal displays an altered phenotype as compared to wild-type animals. In some embodiments, the altered phenotype is the overexpression of mRNA for a LPH3 gene as compared to wild-type levels of LPH3 expression. In other embodiments, the altered phenotype is the decreased expression of MRNA for an endogenous LPH3 gene as compared to wild-type levels of endogenous LPH3 expression. Methods for analyzing the presence or absence of such phenotypes include Northern blotting, mRNA protection assays, and RT-PCR. In other embodiments, the transgenic animal has a knock out mutation of the LPH3 gene. In preferred embodiments, the transgenic animals displays an eye disease phenotype, such a glaucoma, as a result of the knock out mutation.

The transgenic animals of the present invention find use in dietary and drug screens. In some embodiments, the transgenic animals (e.g., animals displaying an eye disease phenotype) are fed test or control diets and the response of the animals to the diets is evaluated. In other embodiments, test compounds (e.g., a drug that is suspected of being useful to treat eye disease) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter which allows reproducible injection of 1-2 picoliters (p1) of DNA solution. The use of zygotes as a target for gene transfer has a major advantage in that in most cases the injected DNA will be incorporated into the host genome before the first cleavage (Brinster et al, Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Patent No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260-1264 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (D. Jahner et al., Proc. Natl. Acad Sci. USA 82:6927-693 [1985]). Transfection is easily and efficiently obtained by culturing the blastomeres on a monolayer of virus-producing cells (Van der Putten, supra; Stewart, et aL, EMBO J., 6:383-388 [1987]). Alternatively, infection can be performed at a later stage. Virus or virus-producing cells can be injected into the blastocoele (D. Jahner et al, Nature 298:623-628 [1982]). Most of the founders will be mosaic for the transgene since incorporation occurs only in a subset of cells which form the transgenic animal. Further, the founder may contain various retroviral insertions of the transgene at different positions in the genome which generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involves the micro-injection of retroviral particles or mitomycin C-treated cells producing retrovirus into the perivitelline space of fertilized eggs or early embryos.

In other embodiments, the transgene is introduced into embryonic stem cells and the transfected stem cells are utilized to form an embryo. ES cells are obtained by culturing pre-implantation embryos in vitro under appropriate conditions. Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal. Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells which have integrated the transgene. This technique obviates the need for growth of the transfected ES cells under appropriate selective conditions prior to transfer into the blastocoel.

In still other embodiments, homologous recombination is utilized knock-out gene function or create deletion mutants. Methods for homologous recombination are described in U.S. Pat. No. 5,614,396, incorporated herein by reference.

II. Compound Screening Using LPH

In some embodiments, the present invention provides methods for screening candidate compounds employing LPH polypeptides and LPH polynucleotides. In certain embodiments, these methods employ a ligand that will bind LPH peptides. In preferred embodiments, the ligand is the TIGR/myocilin peptide. In preferred embodiments, these methods are employed to identify compounds useful in the treatment of eye disease.

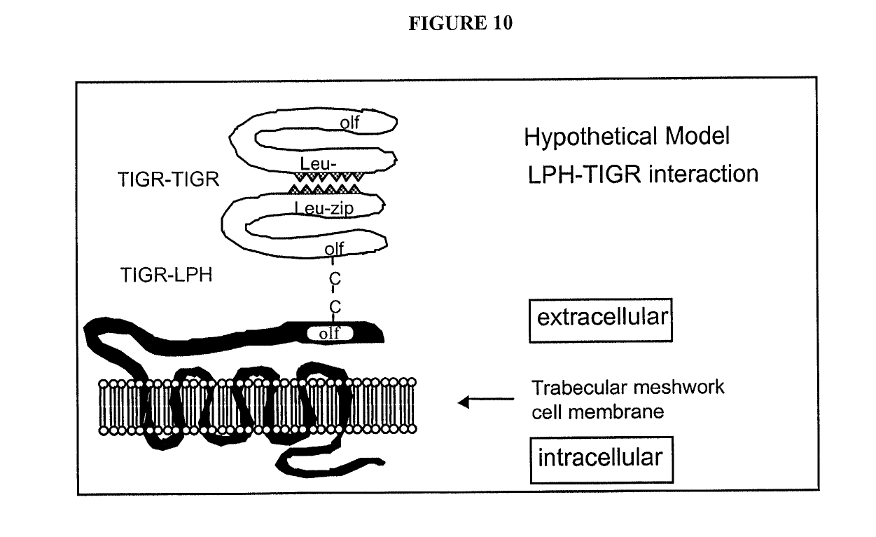

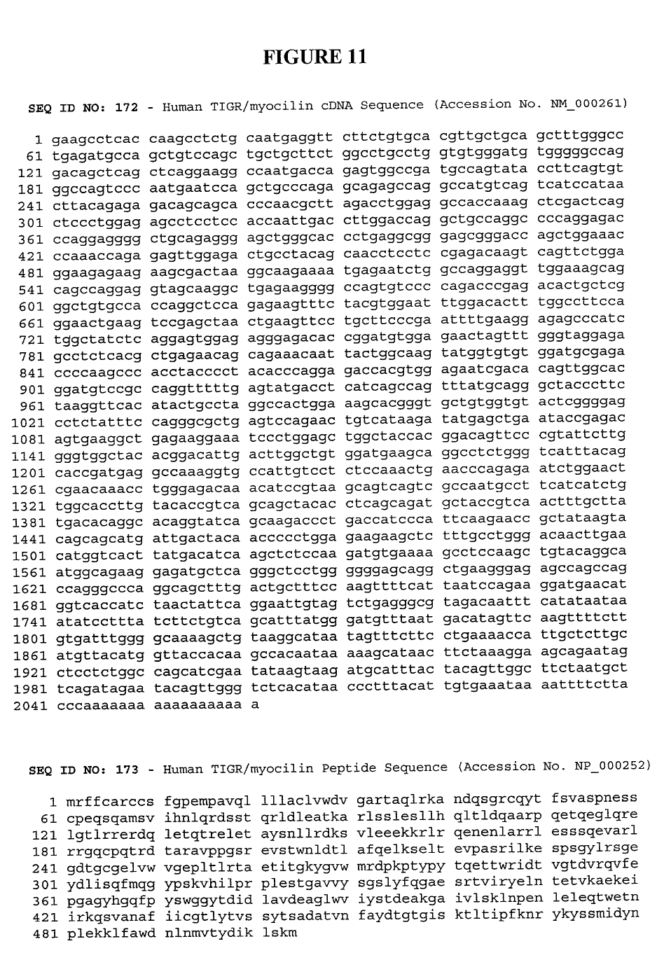

Importantly, the present invention is not limited to a particular mechanism of action. Indeed, an understanding of the mechanism of action is not necessary to practice the present invention. Nevertheless, it is contemplated that LPH peptides (e.g. human LPH3) are expressed by trabecular meshwork as transmembrane proteins, and act as a receptor for the TIGR protein (mutations of which are known to be associated with eye disease). It is further contemplated that the LPH peptide (e.g. human LPH3) normally binds the TIGR peptide via the olfactomedin domains on each protein (see, the hypothetical model in FIG. 10). In this regard, compounds that modulate the interaction of LPH peptides and the TIGR peptide may be useful in treating eye disease (e.g. compounds that promote or inhibit the LPH-TIGR interaction).

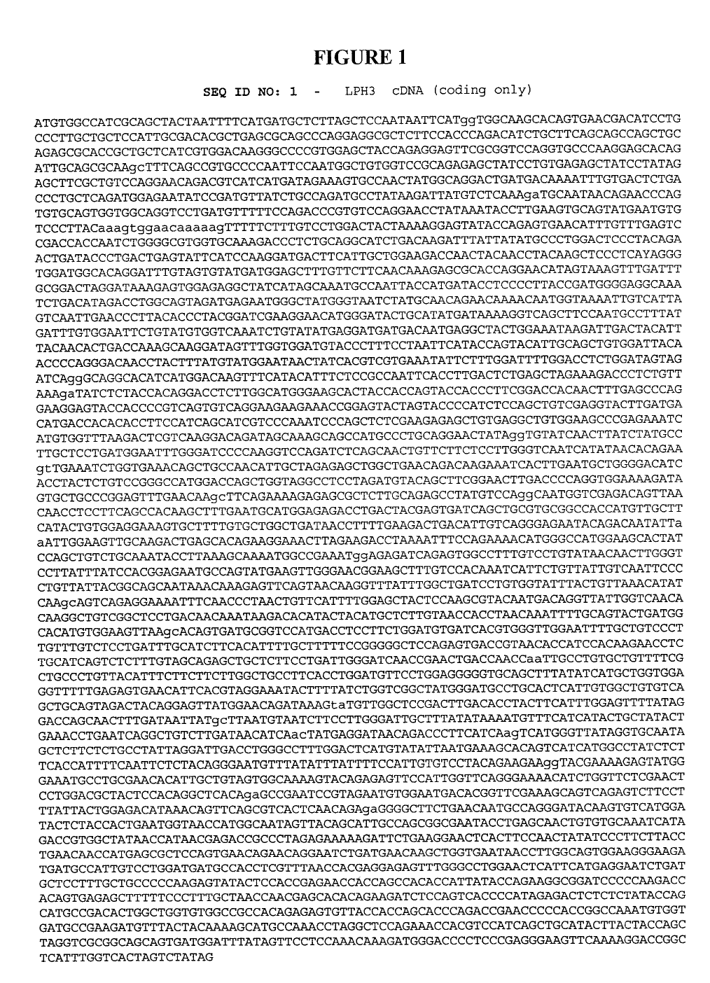

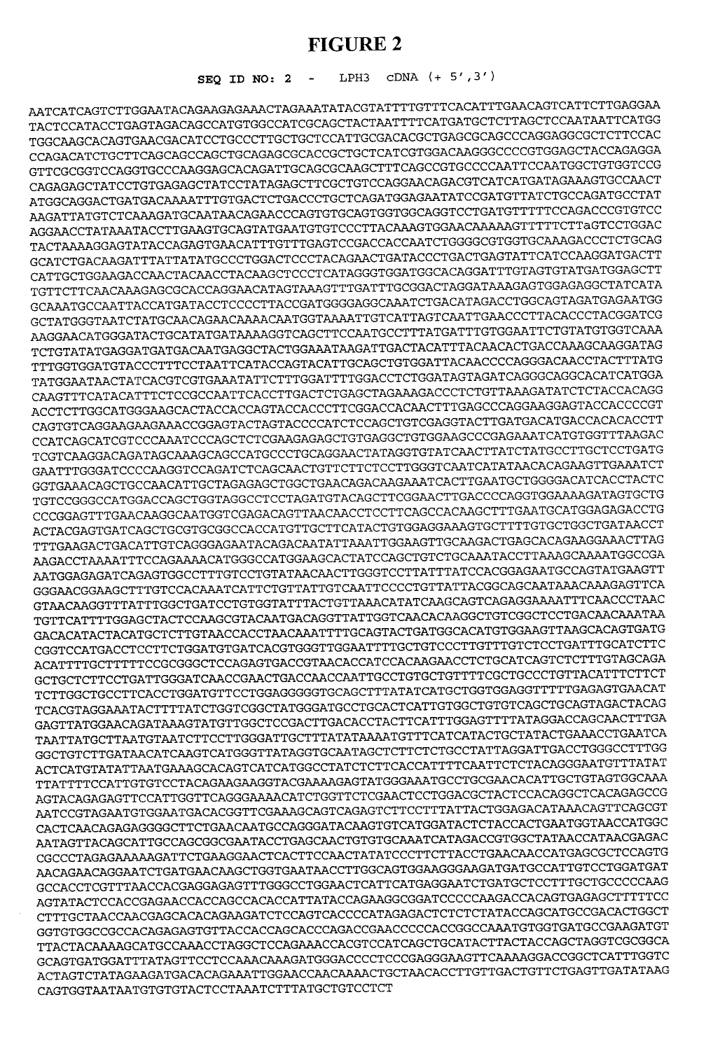

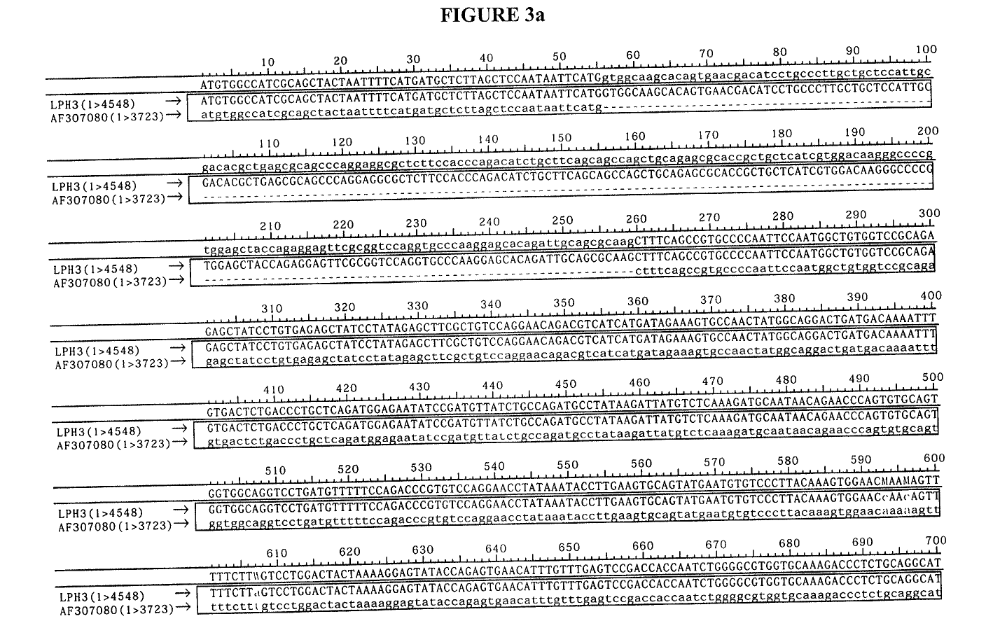

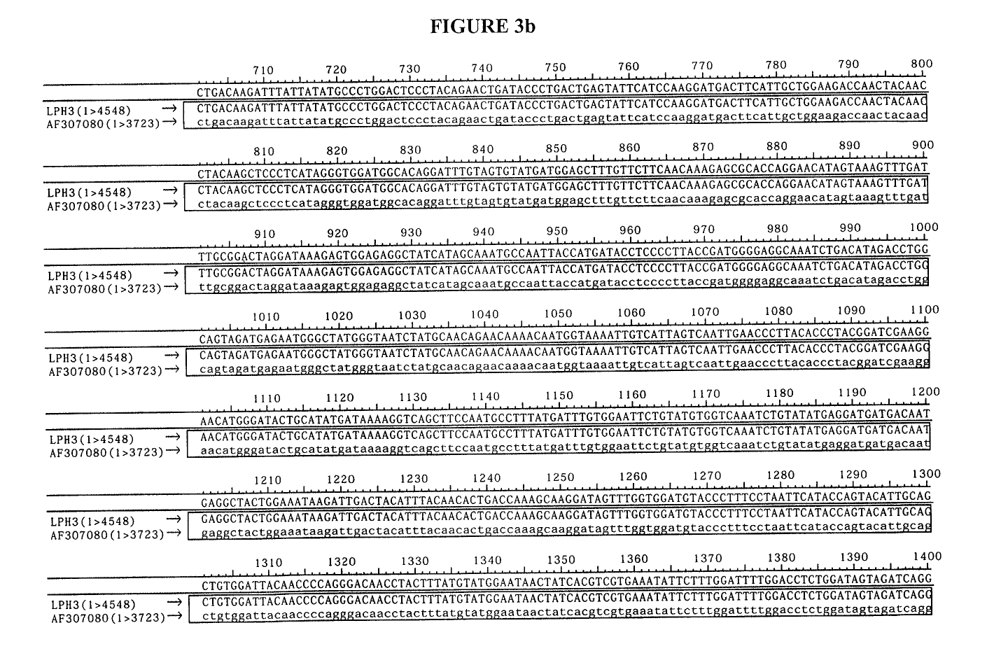

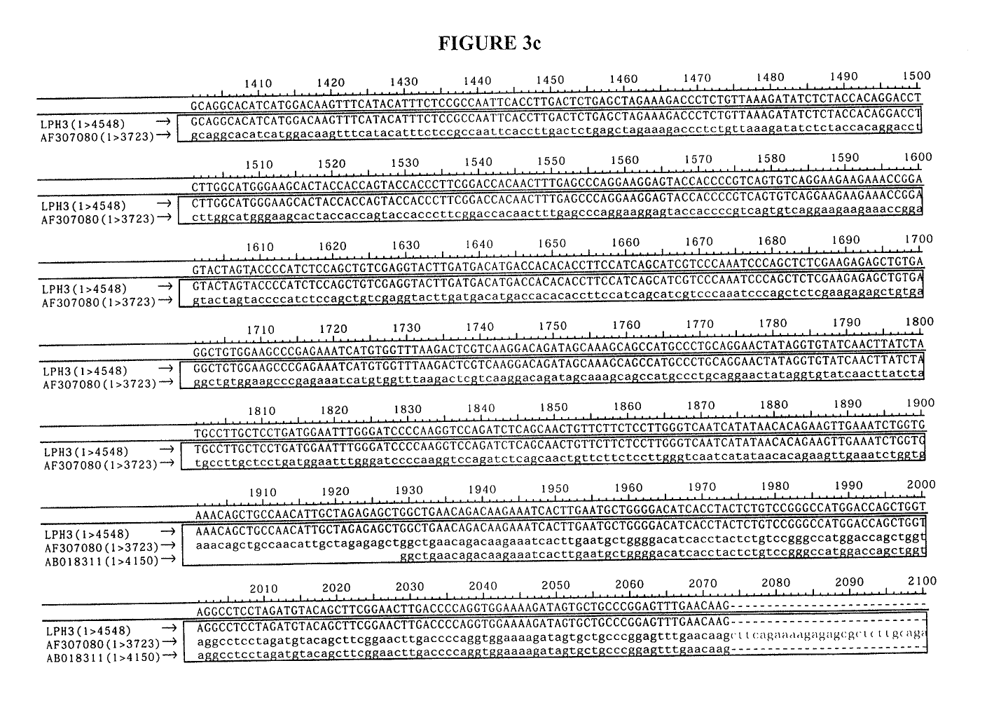

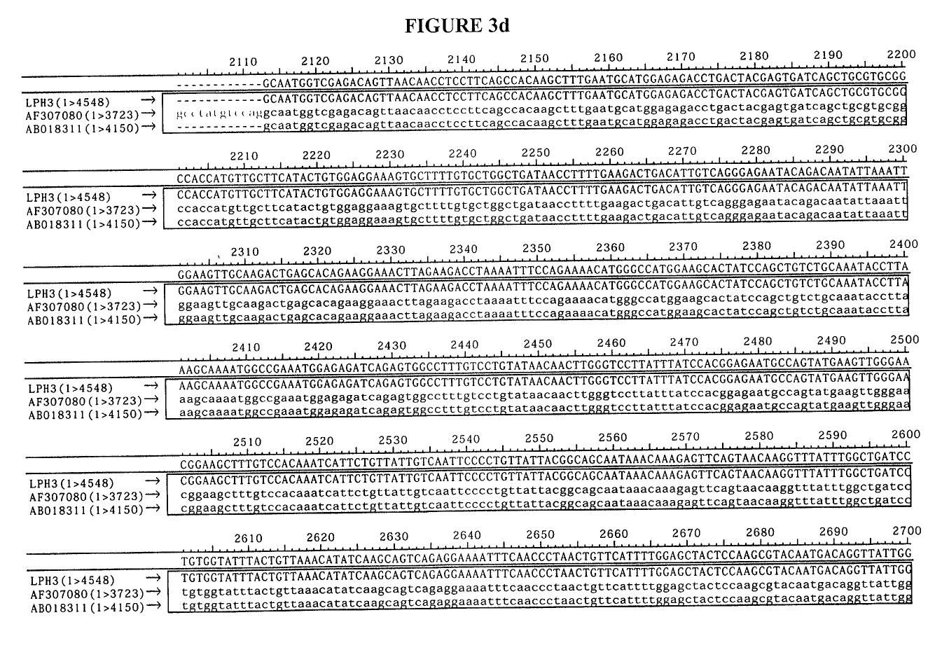

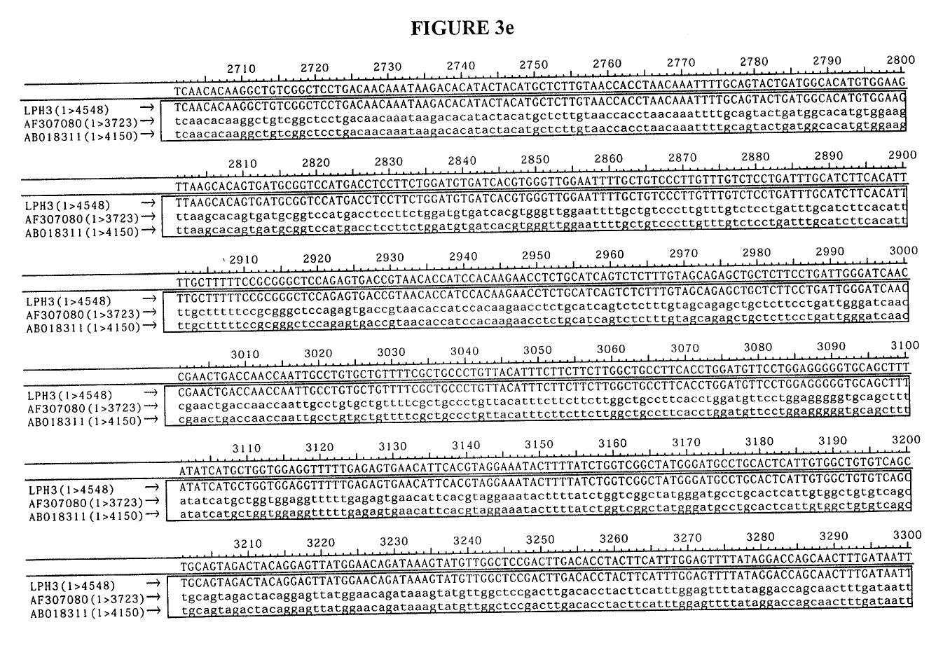

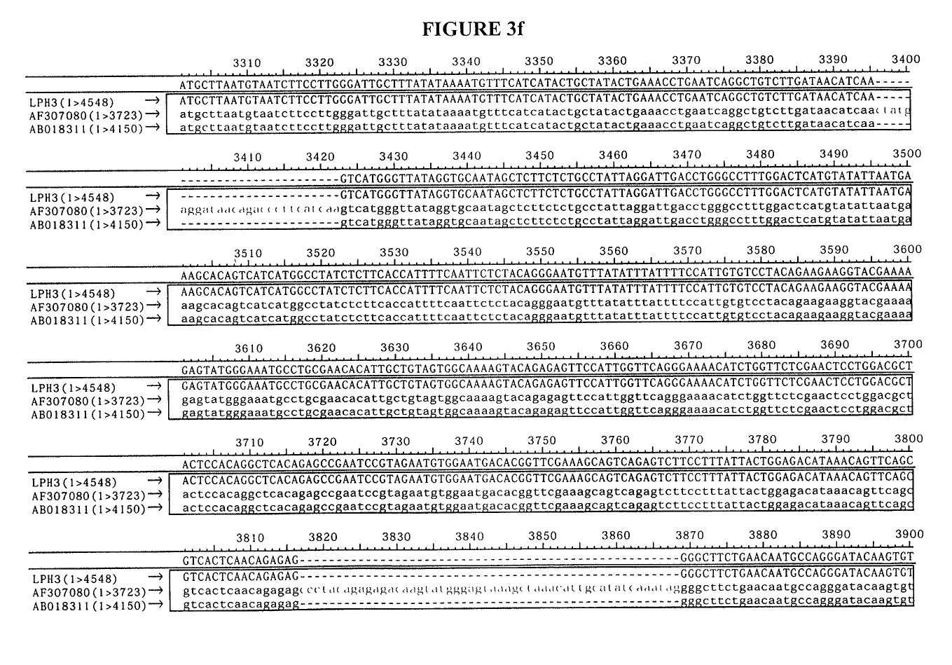

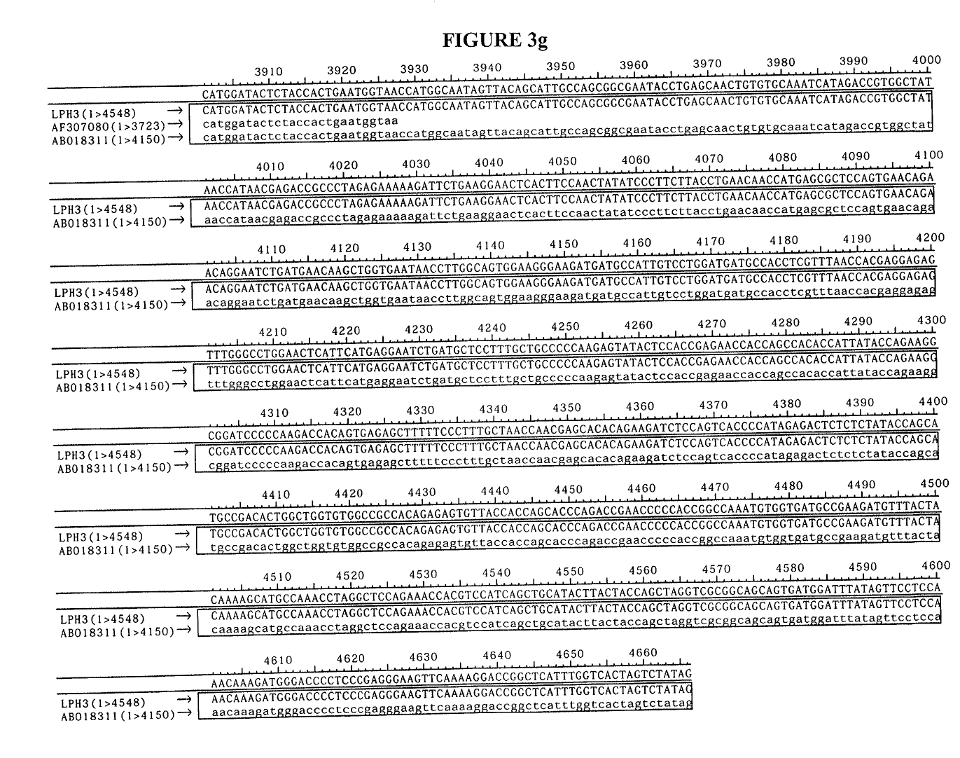

As previously stated, LPH polynucleotides and polypeptides, as well as TIGR polypeptides and polynucleotdies, contain an olfactomedin-like domain. This has been reported by the literature, in, for example, Rozsa, et al., Molecular Vision 1998; 4:20 (e.g., FIG. 7 shows the Olfactomedin domain in human and mouse TIGR, frog, rat, and human olfactomedin-related protein, and rat LPH1); Krasnoperov, et al., Neuron, 18:925-937 (1997), (e.g., FIG. 3B describes presence of the Olfactomedin domain); and Lelianova, et al., Journal of Biological Chemistry, 272:21504-21508. (1997) (e.g., FIG. 1 describes presence of the Olfactomedin domain), all three references herein incorporated by reference.

Specific examples of olfactomedin domains are, for example, exons 3.1 and 4 of human LHP3 (SEQ ID NO:10 and SEQ ID NO:11 respectively, and the peptide or peptides encoded thereby). Another example is the olfactomedin domain of the human TIGR polynucleotide sequence (i.e. nucleotides 758-1528 of SEQ ID NO:172), and the human TIGER polypeptide sequence (i.e. amino acids 246-502 of SEQ ID NO:173). As explained further below, these sequences (i.e. the human LPH3 olfactomedin domain and human TIGR olfactomedin domain) are especially useful in the compound screening methods of the present invention. However, any fragment, variant, homolog, portion, etc, of an LPH polypeptide or polynucleotide (See, Section I. above), is useful in the compound screening methods of the present invention.

In some embodiments, the present invention contemplates in vitro cell-free assays for screening candidate compounds. In particular, the present invention contemplates certain cell-free assays which identify compounds which interfere with or bind to LPH peptides. One method for screening candidate compounds that alter the activity of LPH involves screening compounds for their affinity for LPH polypeptides (e.g. SEQ ID NO:3). Another method involves screening candidate compounds by determining their ability to interfere with the binding of LPH peptides to a ligand. Such ligands are typically labelled with a detectable marker (e.g. enzymes, fluorophores, luminescent molecules, or radio-labelled molecules). Examples of ligands include, but are not limited to, antibodies specific LPH peptides or at least a portion of a TIGR peptide.

Screening compound for their affinity to LPH peptides may be carried out with a simple binding assay. This simple screening assay, as well as those detailed below, may be performed by immobilizing various elements (e.g. proteins, antibodies, candidate compounds) on an immobilization means or solid support (e.g. column, beads, adsorbents, microplate, nitrocellulose paper, etc.), although the present invention contemplates assays conducted without solid supports (e.g. in solution). In one example, an LPH peptides are covalently bound to a column, and detectably labeled candidate compounds are passed over the column. Compound which bind to the LPH peptide are detected and isolated for further characterization (e.g. as compounds useful for the treatment of eye disease). This assay is also performed by immobilizing the candidate compound to the solid support and contacting a detectably labeled LPH peptide with the candidate compound. It is clear that there are many different configurations of labelling and immobilization that find use with the present invention.

The present invention also contemplates various immunoassays for screening candidate compounds. In some embodiments, solid or semi-solid supports may be employed onto which the sample containing LPH peptides has been immobilized. In another method, the sample containing LPH peptides is contacted with a solid support already containing an LPH peptide specific antibody. In both types of assays, a candidate compound is added and the ability of the compound to interfere with antibody binding to the LPH peptide is determined. A wide range of immunoassay techniques are available as can be seen in U.S. Pat. Nos. 4,016,043, and 4,018,653, hereby incorporated by reference. This, of course, includes both single-site and two-site, or “sandwich”, assays of the non-competitive types, as well as in the traditional competitive binding assays.

Sandwich assays are among the most useful and commonly used assays. A number of variations of the sandwich assay technique exist, and all are intended to be encompassed by the present invention. Briefly, in a typical forward assay, an unlabelled LPH peptide specific antibody is immobilized on a solid substrate and a sample comprising the LPH peptide and candidate compound are brought into contact with the bound molecule. After a suitable period of incubation, for a period of time sufficient to allow formation of an antibody-antigen secondary complex, a second antibody specific to the LPH peptide, labelled with a reporter molecule capable of producing a detectable signal is then added and incubated, allowing time sufficient for the formation of a tertiary complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample. Variations on the forward assay include a simultaneous assay, in which the LPH peptide (pre-incubated with the candidate compound) and labelled antibody are added simultaneously to the bound antibody. These techniques are well known to those skilled in the art, including any minor variations as will be readily apparent (See, U.S. Pat. No. 5,948,889, hereby incorporated by reference).

In the typical forward sandwich assay, a first antibody having specificity for an LPH peptide, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking, covalently binding or physically adsorbing, the polymer-antibody complex, which is washed in preparation for the test sample. An aliquot of the sample to be tested is then added to the solid phase complex and incubated at approximately 25° C. for a period of time sufficient to allow binding of any subunit present in the antibody. The incubation period will vary but will generally be in the range of about 2-40 minutes. Following the incubation period, the antibody subunit solid phase is washed and dried and incubated with a second antibody specific for a portion of the LPH peptide. The second antibody is linked to a reporter molecule which is used to indicate the binding of the second antibody.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores, luminescent molecules or radioisotopes.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine, 5-aminosalicyclic acid, or toluidine are commonly used. It is also possible to employ fluorogenic substrates, which yield a fluorescent product rather than the chromogenic substrates noted above. In all cases, the enzyme-labelled antibody is added to the first antibody-LPH peptide complex, allowed to bind, and then the excess reagent is washed away. A solution containing the appropriate substrate is then added to the tertiary complex of antibody-antigen-antibody. The substrate will react with the enzyme linked to the second antibody, giving a qualitative visual signal, which may be further quantitated, usually spectrophotometrically, to give an indication of the amount of LPH peptide that is present in the sample.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-LPH peptide complex. After washing off the unbound reagent, the remaining tertiary complex is then exposed to the light of the appropriate wavelength the fluorescence observed indicates the presence of LPH peptide. Immunofluorescent and EIA techniques are both very well established in the art. However, other reporter molecules, such as radioisotope, chemiluminescent or bioluminescent molecules, may also be employed. It will be readily apparent to one skilled in the art how to vary the procedure to suit the required purpose.

The present invention also contemplates an assay similar to the above immunoassay, except the first or second (or both) antibodies are replaced with a TIGR peptide (e.g. the olfactomedin domain of SEQ ID NO:173, or other portions of a TIGR peptide). In one example, an LPH peptide is bound to a solid support, and a detectably labelled TIGR peptide is contacted with the LPH peptide alone, and in the presence of a candidate compound. The amount of binding is then compared between the two samples. Compounds which prevent the TIGR peptide from binding to the LPH peptide may be useful as eye disease treating compounds. This assay may also be performed with the TIGR peptide bound to a solid support, and contacting detectably labeled LPH peptides with the TIGR peptide in the presence and absence of a candidate compound. In all of these assays, antibody molecules may be used as reporter molecules, or to bind other molecules to solid supports. Similar protein-protein screening assays are detailed in U.S. Pat. No. 5,623,051, hereby incorporated by reference, providing further guidance for the compound screening methods of the present invention.

The present invention contemplates many other means of screening compounds. The examples provided above are presented merely to illustrate a range of techniques available. One of ordinary skill in the art will appreciate that many other screening methods can be used.

In particular, the present invention contemplates the use of cell lines transfected with LPH polynucleotides for screening compounds for activity, and in particular to high throughput screening of compounds from combinatorial libraries (e.g., libraries containing greater than 10⁴ compounds). The cell lines of the present invention can be used in a variety of screening methods. In some embodiments, the cells can be used in second messenger assays that monitor signal transduction following activation of cell-surface receptors. In other embodiments, the cells can be used in reporter gene assays that monitor cellular responses at the transcription/translation level. In still further embodiments, the cells can be used in cell proliferation assays to monitor the overall growth/no growth response of cells to external stimuli.

In second messenger assays, the host cells are preferably transfected as described above with vectors encoding LPH polynucleotides. The host cells are then treated with a compound or plurality of compounds (e.g., from a combinatorial library) and assayed for the presence or absence of a response. It is contemplated that at least some of the compounds in the combinatorial library can serve as agonists, antagonists, activators, or inhibitors of the protein or proteins encoded by the vectors. It is also contemplated that at least some of the compounds in the combinatorial library can serve as agonists, antagonists, activators, or inhibitors of protein acting upstream or downstream of the protein encoded by the vector in a signal transduction pathway. In this regard, compounds useful in the treatment or prevention of eye disease may be identified.

In some embodiments, the second messenger assays measure fluorescent signals from reporter molecules that respond to intracellular changes (e.g., Ca²⁺ concentration, membrane potential, pH, IP₃, cAMP, arachidonic acid release) due to stimulation of membrane receptors and ion channels. Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

In general, the host cells are loaded with the indicator prior to exposure to the compound. In some embodiments, the host cells are exposed to TIGR peptides. Responses of the host cells to treatment with the compounds or TIGR peptides can be detected by methods known in the art, including, but not limited to, fluorescence microscopy, confocal microscopy (e.g., FCS systems), flow cytometry, microfluidic devices, FLIPR systems (See, e.g., Schroeder and Neagle, J. Biomol. Screening 1:75-80 [1996]), and plate-reading systems. In some preferred embodiments, the response (e.g., increase in fluorescent intensity) caused by compound of unknown activity is compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

The cells are also useful in reporter gene assays. Reporter gene assays involve the use of host cells transfected with vectors encoding a nucleic acid comprising transcriptional control elements of a target gene (i.e., a gene that controls the biological expression and function of a disease target) spliced to a coding sequence for a reporter gene. Therefore, activation of the target gene results in activation of the reporter gene product. Examples of reporter genes finding use in the present invention include, but are not limited to, chloramphenicol transferase, alkaline phosphatase, firefly and bacterial luciferases, -galactosidase, -lactamase, and green fluorescent protein. The production of these proteins, with the exception of green fluorescent protein, is detected through the use of chemiluminescent, calorimetric, or bioluminecent products of specific substrates (e.g., X-gal and luciferin). Comparisons between compounds of known and unknown activities may be conducted as described above.

The present invention also contemplates all of the above screening assays (and variations of these assays) in a high throughput format. The high throughput adaptation of these assays will be apparent to those skilled in the art (See, U.S. Pat. No. 5,623,051, and Burbaum et al, 1:72-78, Curr. Opin. Chem. Biol. (1997), both of which are herein incorporated by reference). High throughput assays are particularly useful in the present invention because of the ability to screen hundreds, thousands, and even millions of compounds in a short period of time. Typically, standard assays are miniaturized and automated. An example of miniaturization involves replacing a standard 96-well plate with a 1536-well plate which has much smaller wells. A typical number of compounds that may be screened per day is on the order of 3000-5000 compounds in a cell-free assay (around 200 for cell based assays). Therefore, high through-put screening is a useful tool when combined with the assays of the present invention in identifying useful compounds, such as compounds for treating eye diseases.

The art is aware of many other screening techniques, all of which are contemplated by the present invention (e.g. Capillary Electrophoresis based screening methods, see U.S. Pat. No. 5,783,397, hereby incorporated by reference). Also, screening techniques developed in the future are contemplated which may be employed using the compounds and principles of the present invention.

III. Detecting Variations in LPH polynucleotides

The present invention provides methods, compositions, devices, and kits for screening LPH polynucleotides (both DNA and RNA) and LPH polypeptides for variations (e.g. polymorphisms or mutations), or for screening polynucleotides and polypeptides of sequences related to LPH (e.g. lectin, latrotoxin, and members of the olfactomedin-related family) for variations. In some embodiments, the present invention provides methods, compositions, devices, and kits for screening LPH polynucleotides from a subject sample as a diagnostic for eye disease or susceptibility to eye disease (e.g. POAG). In certain embodiments, the variations related to eye disease prevent the LPH peptide (e.g. the LPH3 peptide shown in SEQ ID NO:3) from binding the TIGR peptide.

The present invention is not limited to a particular mechanism of action. Indeed, an understanding of the mechanism of action is not necessary to practice the present invention. Nevertheless, it is contemplated that LPH peptides (e.g. human LPH3) are expressed by trabecular meshwork as transmembrane proteins, and act as a receptor for the TIGR protein (variations of which are known to be associated with eye disease). It is further contemplated that the LPH peptide (e.g. human LPH3) normally binds the TIGR peptide via the olfactomedin domains on each protein (see, the hypothetical model in FIG. 10). In this regard, variations (e.g. polymorphisms or mutations) in the LPH peptide (e.g. human LPH3) that prevent normal binding to the TIGR peptide are thought to be, at least in part, responsible for eye disease such as POAG. Again, an understanding of why certain polymorphisms or mutations in LPH are associated with eye disease is not necessary to successfully practice the present invention.

Accordingly, the present invention provides methods for determining whether a subject has an increased susceptibility to eye disease by determining whether the individual has a variation in an LPH gene (e.g. a polymorphism or mutation an LPH gene). In other embodiments, the present invention provides methods for providing a prognosis of increased risk for eye disease to an individual based on the presence or absence of a variation in an LPH gene. In preferred embodiments, the variation causes the LPH peptide to fail to bind the TIGR peptide. In some embodiments, the variation prevents a cysteine bond from forming between the TIGR peptide and the LPH peptide. In preferred embodiments, the polynucleotide region of the LPH sequence that encodes the olfactomedin domain is screened for variations (see above discussion of olfactomedin domains). In particularly preferred embodiments, the olfactomedin domain of a human LPH polynucleotide is screened. In other particularly preferred embodiments, the olfactomedin domain from human LPH3 (e.g. Exons 3.1 and 4, SEQ ID NOS:10 and 11 respectively) are screened.

The present invention is not limited to detecting variations in certain LPH genes. Indeed, variations in any LPH gene from any species is contemplated. In some embodiments, the LPH gene is selected from LPH1 (CIRL1), LPH2 (CIRL2), and LPH3 (CIRL3), and splice variants of any of these sequences. In related embodiments, the gene is a gene related to LPH genes such as human LEC3 (accession No. AF307080). In some embodiments, the LPH gene is from a cow, rat, dog, cat, horse, pig, monkey, goat, etc. In preferred embodiments, the LPH gene is from a human (e.g. SEQ ID NO:1, 2, and 5). In particular embodiments, the LPH polynucleotide is LPH mRNA (e.g. SEQ ID NO:1 where the T's are replaced by U's).

In particular embodiments, the LPH gene is from a cow, and is selected from the following accession numbers; LPH1-AF111097, AF111098; LPH2-AF111069, AF111070, AF111071, AF111072, AF111073, AF111074, AF111075, AF111076, AF111077, AF111078, AF111079, AF111080, AF111081, AF111082, AF111083, and AF 111084; and LPH3-AF111085, AF111086, AF111087, AF111088, AF111089, AF111090, AF111091, AF111092, AF111093, AF111094, AF111095, and AF11096; or similar sequences from other species. In other embodiments, the LPH gene is from a rat, and is selected from the following accession numbers; LPH1-NM_{—}022962, AF081146, U72487, AF111099, U78105, AF081144, AF081145, AF081146, and AF081147; LPH2-AF063102, AF081148, AF081149, AF081150, AF081151, AF081151, AF081152, and AF081153; and LPH3-AF063103, AF081154, AF081155, AF081156, AF081157, AF081158, and AF081159; or similar sequences from other species.

In preferred embodiments, a sample from a subject thought susceptible to eye disease is tested. In other embodiments, a sample testing is compulsory (e.g., at birth, in utero, for school admission, etc.). In some embodiments, the DNA or RNA tested is from a patient's tissues, organs, or body fluids, including, but not limited to, saliva, blood, urine, tears, or nasal mucous, as well as from other cell samples obtained from that individual including, but not limited to, pathology samples, blood samples, cord blood, umbilical samples, placenta buccal mucosa scrapings, semen, hair, nail pairings, shed skin or cells obtained by chorionic villus sampling, amniocentesis, or other prenatal samples.

In preferred embodiments, patient samples are from patients with glaucoma, patients with glaucoma-related characteristics such as ocular hypertension, relatives of such individuals, individuals at risk of glaucoma, and other individuals deemed suitable for glaucoma screening including members of the general population as follows. Individuals may be screened because of obvious risk, such as presence of glaucoma, family history of glaucoma or presence of glaucoma-associated characteristics such as ocular hypertension, suspicious optic discs, anterior segment dysgenesis, pigment dispersion, pseudoexfoliation, aniridia, increased intraocular pressure, or presence of other developmental or genetic defects associated with glaucoma. Additional categories of individuals to be screened include individuals with other characteristics or health problems that are risk factors for glaucoma, individuals with diabetes, systemic hypertension, individuals over age 40, individuals being treated with medications that can lead to steroid glaucoma, individuals at risk of other iatrogenic forms of glaucoma, and others at risk as identified in the literature.

Patient samples may also be screened for forensic purposes, familial relationship testing, or anthropological samples in addition to medical diagnostic and public health testing situations. This includes screening of samples from individuals or pooled sets of samples from multiple individuals.

Screening LPH or LPH related sequences is not only useful in diagnosing disease (e.g. eye disease), but also may be used for generating other information of clinical use in assessment of risk or in diagnosis or treatment design for glaucoma or glaucoma related characteristics including ocular hypertension. Thus, in addition to genetic testing aimed at determining whether someone has a variation (e.g. mutation or polymorphism) that causes glaucoma, this testing would also include genetic testing aimed at identifying other important pieces of information such as whether one treatment is to be preferred over another, assessment of likely age at onset of disease or specific disease-related characteristics, assessment of risk of reduced- or non-penetrance, assessment of risk of other associated ocular or systemic disease, or other modulation of glaucoma.

A number of methods are available for analysis of sequence variations (e.g. polymorphisms and mutations). Assays for detecting polymorphisms or mutations fall into several categories, including, but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following are examples of methods, assays, devices, kits, and compositions that are useful in the present invention for detecting variations in LPH polynucleotides, which may be, for example, gel-based, capillary-based, filter-based, slide-based, liquid-based, gas-phase, or chip-based systems.

Specific methods for variation detection include DNA sequence determination (Sanger method with either radionuclide or fluorescence label, Maxam and Gilbert Method, HPLC, or other methods) through use of manual, semi-automated, or automated processes including use of gels or capillaries, Mass Spec-based technologies including MALDI-TOF MS, spectral assays, technologies aimed at detection of different melting temperatures for sequences that differ from each other, technologies based on use of confocal or de-convoluting microscopy to evaluate fluorescence labeled DNA, RNA, or DNA/RNA hybrids, via affinity sensor using surface plasmon resonance technologies, PCR and RT-PCR, use of other clone-based, polymerase-based, or PCR based technologies to detect hybridization in simplex or multiplex technologies, electronically active microfabricated array technologies such as APEX microchips, electrical field denaturation, microplate array diagonal gel electrophoresis (MADGE), amplification including TAQMAN technologies, measurements of sequence-base changes in electron transfer through the DNA helix, gene-chip assays involving high density oligonucleotide arrays including Affymetrix-like technologies, microarray technologies, ribotyping, pulsed-field gel electrophoresis (PFGE), field alternation gel electrophoresis (FAGE) and related technologies, allele-specific or differential PCR amplification, hybridization-based technologies including Southems, Northems, dot-blots and slot-blots, double- or triple-helix formation, dual-label fluorescence assays of differential hybridization of LPH DNA, amplified fragment length polymorphism-based fingerprinting (AMF), confirmation variation based technologies such as single strand confirmation polymorphism (SSCP) or GC-clamped denaturing gradient gel electrophoresis or other variations on this concept that evaluate conformational differences in DNA, RNA, or DNA/RNA hybrid structures, other rapid scanning technologies based on screening for sequence mismatches through detection of DNA, RNA, or DNA/RNA hybrids containing mismatched or altered base-pairing including use of altered mobility through capillaries or acrylamide-based, agarose based, MDE or other forms of gel technology, additional rapid scanning technologies based on detection of DNA, RNA, or DNA/RNA hybrids with mismatched or altered base pairing via cleavage at, or other form of digestion of the target through either enzymatic or chemical methods, or polymerase-based extension from, mismatched or altered base pairing of DNA, RNA, or DNA/RNA hybrids, primer-extension-based technologies, allele specific oligonucleotide hybridization, or cutting of cloned or PCR-amplified segments of the gene with restriction enzymes. This use of sequences in screening includes not only use of coding sequences but also use of intronic sequences, flanking portions of unprocessed and processed transcripts, adjoining regions including promoters and other sequences 3′ and 5′ to the gene. For a review of detection methodologies, see Shi, Clinical Chemistry, 47:2, 164-172 [2001], discussing, for example, TAQMAN, Rolling Circle, and INVADER assays, this review hereby incorporated by reference, see also, U.S. Pat. Nos. 6,150,104 and 6,210,884 for suitable detection methods, both of which are hereby incorporated by reference).

These techniques may be employed to find new variations (e.g. polymorphisms or mutations), or to detect already known variations of interest. Certain exemplary techniques are explained in more detail below.

A. Direct sequencing Assays

In some embodiments of the present invention, variations are detected using a direct sequencing technique. In these assays, DNA samples are first isolated from a subject (e.g. a patient suspected of developing glaucoma or a patient with glaucoma) using any suitable method. In some embodiments, the region of interest (e.g. olfactomedin domain from human LPH3) is cloned into a suitable vector and amplified by growth in a host cell (e.g., a bacteria). In other embodiments, DNA in the region of interest is amplified using PCR (e.g. utilizing primers from intronic regions surrounding exon 4 of human LPH3, see FIG. 7x). Importantly, FIG. 7 (genomic human LPH3) and FIG. 9 (genomic human LPH1) allow one skilled in the art to design primers that flank the exon region by employing the intronic sequences that surround each exon. In this regard, the full exon regions may be amplified prior to being screened for mutations.

Following amplification, DNA in the region of interest (e.g., the region containing the variation of interest, such as a known SNP) is sequenced using any suitable method, including but not limited to manual sequencing using radioactive marker nucleotides, or automated sequencing. The results of the sequencing are displayed using any suitable method. The sequence is examined and the presence or absence of a given variation, such as a SNP or mutation, is determined.

B. PCR Assays

In some embodiments of the present invention, LPH variations are detected using a PCR-based assay. In some embodiments, the PCR assay comprises the use of oligonucleotide primers that hybridize only to the mutant or wild type allele of LPH1, LPH2, or LPH3 (e.g., to the region of polymorphism). Both sets of primers are used to amplify a sample of DNA. If only the mutant primers result in a PCR product, then the patient has the mutant LPH allele. If only the wild-type primers result in a PCR product, then the patient has the wild type allele of LPH.

C. Fragment Length Polymorphism Assays

In some embodiments of the present invention, LPH variations are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a variation, such as a SNP or a mutation, will have a different banding pattern than wild type.

1. RFLP Assay

In some embodiments of the present invention, LPH variations are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

2. CFLP Assay

In other embodiments, LPH variations are detected using a CLEAVASE fragment length polymorphism assay (CFLP; Third Wave Technologies, Madison, Wis.; See e.g., U.S. Pat. Nos. 5,843,654 and 5,888,780; both of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

D. Hybridization Assays

In preferred embodiments of the present invention, LPH variations are detected by a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

1. Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labelled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labelled probe.

2. Detection of Hybridization Using “DNA Chip” Assays

In some embodiments of the present invention, LPH variations are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996, and 5,858,659; both of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; and 6,051,380; both of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311, herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

3. Enzymatic Detection of Hybridization

In some embodiments of the present invention, genomic profiles are generated using a assay that detects hybridization by enzymatic cleavage of specific structures (INVADER assay, Third Wave Technologies; See e.g., U.S. Pat. Nos. 5,846,717; 6,090,543; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified genomic DNA. The isolated DNA sample is contacted with the first probe specific either for a SNP/mutation or wild type sequence and allowed to hybridize. Then a secondary probe, specific to the first probe, and containing the fluorescein label, is hybridized and the enzyme is added. Binding is detected by using a fluorescent plate reader and comparing the signal of the test sample to known positive and negative controls.

In some embodiments, hybridization of a bound probe is detected using a TAQMAN assay (PE Biosystems, Foster City, Calif.; See e.g., U.S. Pat. Nos. 5,962,233 and 5,538,848, each of which is herein incorporated by reference). The assay is performed during a PCR reaction. The TAQMAN assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, LPH variations are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labelled antibody specific for biotin).

E. Mass Spectroscopy Assays

In some embodiments, a MassARRAY system (Sequenom, San Diego, Calif.) is used to detect LPH variations (See e.g., U.S. Pat. Nos. 6,043,031; and 5,605,798; both of which is herein incorporated by reference). DNA is isolated from patient samples (e.g. blood samples) using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

F. Mutant Analysis by Differential Antibody Binding

In other embodiments of the present invention, antibodies (See above for antibody production) are used to determine if an individual contains an allele encoding a LPH gene containing a mutation or polymorphism. In preferred embodiments, antibodies are utilized that discriminate between mutant (e.g., truncated proteins); and wild-type proteins. In some particularly preferred embodiments, the antibodies are directed to the C-terminus of LPH3. In other preferred embodiments, the antibodies are directed toward the olfactomedin domain of LPH (e.g. exons 3.1 and 4 of human LPH3). Methods for detecting LPH mutations employing antibodies or LPH peptides includes, but is not limited to, gel and capillary based systems, chemical and enzymatic cleavage assays, ELISAs, detection with antibodies in Western or dot-blots, immunoprecipitation, immunodetection, in assays for binding of LPH peptides to the TIGR/myocilin protein, in yeast hybrid assays of LPH protein interaction with other proteins, dual label fluorescense color based assay of protein-protein interactions or co-localizations, use of epitope-tagged or fusion variants of LPH peptides, and flow cytometry.

G. Kits for Analyzing Risk of Eye Disease

The present invention also provides kits for determining whether an individual contains a wild-type or mutant allele of LPH (e.g. human LPH3). In some embodiments, the kits are useful determining whether the subject is at risk of developing eye disease, such as POAG. The diagnostic kits are produced in a variety of ways. In some embodiments, the kits contain at least one reagent for specifically detecting a mutant LPH allele or protein (e.g. nucleic acid probes or primers, or LPH specific antibodies). In preferred embodiments, the kits contains reagents for detecting a SNP in the olfactomedin domain of a human LPH gene (e.g. human LPH3). In preferred embodiments, the reagent is a nucleic acid that hybridizes to nucleic acids containing the SNP and that does not bind to nucleic acids that do not contain the SNP. In other preferred embodiments, the reagents are primers for amplifying the region of DNA containing the SNP. In still other embodiments, the reagents are antibodies which preferentially bind either the wild-type or truncated LPH proteins. In some embodiments, the kit contains instructions for performing the assay and determining whether the subject is at risk for developing eye disease. In preferred embodiments, the instructions specify that risk for developing eye disease is determined by detecting the presence or absence of a mutant LPH allele in the subject. In some embodiments, the kits include ancillary reagents such as buffering agents, nucleic acid stabilizing reagents, protein stabilizing reagents, and signal producing systems (e.g., florescence generating systems as Fret systems). The test kit may be packages in any suitable manner, typically with the elements in a single container or various containers as necessary along with a sheet of instructions for carrying out the test. In some embodiments, the kits also preferably include a positive control sample.

H. Devices for Detecting Polymorphisms

In certain embodiments, the present invention provides a mutation detecting device. In some embodiments, the mutation detecting device comprises at least a portion of an LPH polynucleotide, wherein the detecting device is configured to receive a patient sample. In certain embodiments, the mutation detecting device is a microchip. In other embodiments, the mutation detecting device is a DNA chip. In some embodiments, the mutation detecting device is an injection molded plastic cartridge. In some embodiments, the mutation detecting device comprises at least two LPH polynucleotides, wherein one of the LPH polynucleotides comprises a mutation diagnostic for an eye disease, and wherein one of the LPH polynucleotides comprises at least a portion of a wild type LPH polynucleotide. In some embodiments, the mutation detecting device further comprises one or more probes or primers capable of hybridizing to an LPH polynucleotide. In particular embodiments, the mutation detecting device further comprises buffers and related agents for performing a detecting assay with the device.

I. BioInformatics and Related Techniques

The present invention also contemplates the use of bioinformatics techniques to identify variations in LPH polynucleotides like, for example, mutations that contribute to eye disease. Examples of such bioinformatic techniques are found in U.S. Pat. Nos. 6,141,657, and 5,972,693 assigned to CuraGen Corporation, both of which are hereby incorporated by reference. In some embodiments, the present invention provides methods of determining an individual's risk of developing eye disease based on the presence of one or more mutant alleles of an LPH sequence. In some embodiments, the analysis of polymorphism data is automated. For example, in some embodiments, the present invention provides a bioinformatics research system comprising a plurality of computers running a mulit-platform object oriented programming language (See e.g., U.S. Pat. No. 6,125,383; herein incorporated by reference). In some embodiments, one of the computers stores genetics data (e.g., the risk of contacting eye disease associated with a given polymorphism). In some embodiments, one of the computers stores application programs (e.g., for analyzing transission disequalibrium data or determining genotype relative risks and population attributable risks. Results are then delivered to the user (e.g., via one of the computers or via the internet).

Related methods for finding mutations in LPH polynucleotides that are related to disease, such as eye disease, are found in U.S. Pat. No. 6,146,828, assigned to Exact Sciences, hereby incorporated by reference, where genetic testing and statistical analysis are employed to find disease causing mutations or identify a patient sample as containing a disease causing mutation.

IV. LPH Therapeutics

A. Gene Therapy Using LPH

The present invention also provides methods and compositions suitable for gene therapy to, for example, alter LPH expression, production, or function, as a means for treating disease (e.g. eye disease). In some embodiments, gene therapy is performed by providing a subject with a wild-type allele of LPH3 (e.g., SEQ ID NOS:1, 2, and 5, or fragments or variants thereof as explained above). In other embodiments, gene thereapy is performed by providing a subject with a wild-type allele of LPH1 or LPH2. In certain embodiments, antisense molecules are expressed in subject to block the expression of an LPH gene. In certain embodiments, this therapy prevents or reduces the symptoms associated with eye disease. Subjects in need of such therapy are identified by the methods described above. As described above, LPH3 is expressed in trabecular meshwork cells in the eye. Accordingly, a preferred method of gene therapy is to provide the LPH sequences in a suitable gene therapy vector with promoters specific for this area of the eye.

Viral vectors commonly used for in vivo or ex vivo targeting and therapy procedures are DNA-based vectors and retroviral vectors. Methods for constructing and using viral vectors are known in the art (See e.g., Miller and Rosman, BioTech., 7:980-990 [1992]). Preferably, the viral vectors are replication defective, that is, they are unable to replicate autonomously in the target cell. In general, the genome of the replication defective viral vectors that are used within the scope of the present invention lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (i.e., on the isolated DNA) or in situ, using the techniques of genetic manipulation or by treatment with mutagenic agents.

Preferably, the replication defective virus retains the sequences of its genome that are necessary for encapsidating the viral particles. DNA viral vectors include an attenuated or defective DNA viruses, including, but not limited to, herpes simplex virus (HSV), papillomavirus, Epstein Barr virus (EBV), adenovirus, adeno-associated virus (AAV), and the like. Defective viruses, that entirely or almost entirely lack viral genes, are preferred, as defective virus is not infective after introduction into a cell. Use of defective viral vectors allows for administration to cells in a specific, localized area, without concern that the vector can infect other cells. Thus, a specific tissue can be specifically targeted (e.g. the eye of a mammal). Examples of particular vectors include, but are not limited to, a defective herpes virus 1 (HSV1) vector, defective herpes virus vector lacking a glycoprotein L, or other defective herpes virus vectors; an attenuated adenovirus vector, such as the vector described by Stratford-Perricaudet et al. (J. Clin. Invest., 90:626-630 [1992]; See also, La Salle et al., Science 259:988-990 [1993]); and a defective adeno-associated virus vector (Samulski et al., J. Virol., 61:3096-3101 [1987].

Preferably, for in vivo administration, an appropriate immunosuppressive treatment is employed in conjunction with the viral vector (e.g., adenovirus vector), to avoid immuno-deactivation of the viral vector and transfected cells. For example, immunosuppressive cytokines, such as interleukin-12 (IL-12), interferon-gamma (IFN- ), or anti-CD4 antibody, can be administered to block humoral or cellular immune responses to the viral vectors. In addition, it is advantageous to employ a viral vector that is engineered to express a minimal number of antigens.

In a preferred embodiment, the vector is an adenovirus vector. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to type 2 or type 5 human adenoviruses (Ad 2 or Ad 5), or adenoviruses of animal origin (See e.g., WO94/26914). Those adenoviruses of animal origin that can be used within the scope of the present invention include adenoviruses of canine, bovine, murine, ovine, porcine, avian, and simian (e.g., SAV) origin. Preferably, the adenovirus of animal origin is a canine adenovirus, more preferably a CAV2 adenovirus (e.g. Manhattan or A26/61 strain (ATCC VR-800)).

Preferably, the replication defective adenoviral vectors of the invention comprise the ITRs, an encapsidation sequence and the nucleic acid of interest. Still more preferably, at least the E1 region of the adenoviral vector is non-functional. The deletion in the E1 region preferably extends from nucleotides 455 to 3329 in the sequence of the Ad5 adenovirus (PvuII-BgIII fragment) or 382 to 3446 (HinfII-Sau3A fragment). Other regions may also be modified, in particular the E3 region (e.g., WO95/02697), the E2 region (e.g., WO94/28938), the E4 region (e.g., WO94/28152, WO94/12649 and WO95/02697), or in any of the late genes L1-L5.

In a preferred embodiment, the adenoviral vector has a deletion in the E1 region (Ad 1.0). Examples of E1-deleted adenoviruses are disclosed in EP 185,573 the contents of which are incorporated herein by reference. In another preferred embodiment, the adenoviral vector has a deletion in the E1 and E4 regions (Ad 3.0). Examples of E1/E4-deleted adenoviruses are disclosed in WO95/02697. In still another preferred embodiment, the adenoviral vector has a deletion in the E1 region into which the E4 region and the nucleic acid sequence are inserted.

The replication defective recombinant adenoviruses according to the invention can be prepared by any technique known to the person skilled in the art (See e.g., Levrero et al., Gene 101:195 [1991]). In particular, they can be prepared by homologous recombination between an adenovirus and a plasmid which carries, inter alia, the DNA sequence of interest (e.g. SEQ ID NO:1). The homologous recombination is accomplished following co-transfection of the adenovirus and plasmid into an appropriate cell line. The cell line that is employed should preferably (i) be transformable by the elements to be used, and (ii) contain the sequences that are able to complement the part of the genome of the replication defective adenovirus, preferably in integrated form in order to avoid the risks of recombination. Examples of cell lines that may be used are the human embryonic kidney cell line 293, which contains the left-hand portion of the genome of an Ad5 adenovirus (12%) integrated into its genome, and cell lines that are able to complement the E1 and E4 functions, as described in applications WO94/26914 and WO95/02697. Recombinant adenoviruses are recovered and purified using standard molecular biological techniques, that are well known to one of ordinary skill in the art.

The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The use of vectors derived from the AAVs for transferring genes in vitro and in vivo has been described (See e.g., U.S. Pat. No. 4,797,368; and U.S. Pat. No., 5,139,941, both of which are herein incorporated by reference). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In another embodiment, the gene can be introduced in a retroviral vector (e.g., as described in U.S. Pat. Nos. 5,399,346, and 4,650,764; both of which are herein incorporated by reference). The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus.

In general, in order to construct recombinant retroviruses containing a nucleic acid sequence, a plasmid is constructed that contains the LTRs, the encapsidation sequence and the coding sequence. This construct is used to transfect a packaging cell line, which cell line is able to supply in trans the retroviral functions that are deficient in the plasmid. In general, the packaging cell lines are thus able to express the gag, pol and env genes. Such packaging cell lines have been described in the prior art, in particular the cell line PA317 (U.S. Pat. No. 4,861,719, herein incorporated by reference), the PsiCRIP cell line (See, WO90/02806), and the GP+envAm-12 cell line (See, WO89/071 50). In addition, the recombinant retroviral vectors can contain modifications within the LTRs for suppressing transcriptional activity as well as extensive encapsidation sequences that may include a part of the gag gene (Bender et al., J. Virol., 61:1639 [1987]). Recombinant retroviral vectors are purified by standard techniques known to those having ordinary skill in the art.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes. Particularly useful lipid compounds and compositions for transfer of nucleic acids are described in U.S. Pat. No. 5,459,127, herein incorporated by reference.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce the vector in vivo as a naked DNA plasmid. Methods for formulating and administering naked DNA to mammalian muscle tissue are disclosed in U.S. Pat. Nos. 5,580,859 and 5,589,466, both of which are herein incorporated by reference.

DNA vectors for gene therapy can be introduced into the desired host cells by methods known in the art, including but not limited to transfection, electroporation, microinjection, transduction, cell fusion, DEAE dextran, calcium phosphate precipitation, use of a gene gun, or use of a DNA vector transporter. Receptor-mediated DNA delivery approaches can also be used (Curiel et al., Hum. Gene Ther., 3:147-154 [1992]).

B. Pharmaceutical Compositions Relating to LPH

The present invention further provides pharmaceutical compositions which may comprise all or portions of an LPH polynucleotide sequence, LPH polypeptide, inhibitor or antagonist of LPH bioactivity, including antibodies and antisense sequences, alone or in combination with at least one other agent, such as a stabilizing compound, and may be administered in any sterile, biocompatible pharmaceutical carrier, including, but not limited to, saline, buffered saline, dextrose, and water. In preferred embodiments, these pharmaceutical compositions are administered to reduce or eliminate the symptoms of eye disease, such as POAG or occular hypertension.

The present invention contemplates the administration of LPH (e.g. human LPH3) peptides to patients suffering from eye disease. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, intraperitoneal, and in preferred embodiments, optically. Therapeutic administration of a polypeptide intracellularly can also be accomplished using gene therapy as described above.

In some embodiments, the pharmaceutical compositions contain compounds (e.g. drugs) identified by the methods described in part II. above. For example, the pharmaceutical compositions of the present invention may contain compounds found by screening candidate compound for their ability to promote the binding of TIGR to LPH (e.g. LPH3), or inhibit the binding of TIGR to LPH, or compounds that mimic the TIGR peptide, or mimic the LPH3 peptide, or mimic the ability of LPH peptides to bind the TIGR peptide.

As is well known in the medical arts, dosages for any one patient depends upon many factors, including the patient's size, body surface area, age, the particular compound to be administered, sex, time and route of administration, general health, and interaction with other drugs being concurrently administered.

Accordingly, in some embodiments of the present invention, LPH nucleotides and LPH amino acid sequences, or other compounds identified by the methods of the present invention, can be administered to a patient alone, or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert. In another embodiment of the present invention, LPH polynucleotide sequences or LPH amino acid sequences may be administered alone to individuals subject to or suffering from a disease, such as POAG or occular hypertension.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, intranasal administration or optical administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In preferred embodiments of the present invention, pharmaceutical compositions are administered to a patient in the form of opthalmic solutions adapted for topical administration to the eye, such as solutions, suspensions, ointments and solid inserts. Examples of such solutions are presented in U.S. Pat. Nos. 5,049,587 and 5,688,770, both of which are hereby incorporated by reference. In particularly preferred embodiments, the LPH related pharmaceutical compositions of the present invention are administered to glaucoma patients in solutions containing intraocular pressure lessening agents, and other eye disease treating agents (See, e.g. U.S. Pat. Nos. 5,049,587 and 4,617,299, both of which are hereby incorporated by reference.)

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount of LPH3 polynucleotides, or LPH polypeptides may be that amount that competively inhibits TIGR peptide from binding trabecullar meshwork cells (e.g. in the case where a patient expresses excessive TIGR protein in the eye). Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Pharmaceutical preparations which can be used orally include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition, such as glaucoma or occular hypertension.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration.

A therapeutically effective dose refers to that amount of LPH3 peptides, antibodies, antisense molecules, LPH antagonists or agonists, etc, which ameliorates symptoms of an eye disease. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds which exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference).

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); tmol (micromoles); nmol (nanomoles); pmol (picomoles); g (grams); mg (milligrams); tg (micrograms); ng (nanograms); 1 or L (liters); ml (milliliters); μl (microliters); cm (centimeters); mm (millimeters); μm (micrometers); nm (nanometers); DS (dextran sulfate); C (degrees Centigrade); and Sigma (Sigma Chemical Co., St. Louis, Mo.).

### Example 1

### BioInformatic Based Construction of the Human LPH3 Gene

This example describes in general terms the use of bioinformatic techniques to construct the LPH3 polynucleotide sequence of the present invention. Pre-existing human and mammalian sequences were utilized to query sequence databases using BLASTN 2.1.2 (Nov-13-2000 version, Altschul, etal., Nucleic Acids Res. 25:3389-3402). Only human sequences were compiled to construct the human LPH3 sequences of the present invention. Three sequences were then used as initial probes.

One of these sequences was a partial human LPH3 MRNA sequence (accession number AB018311, a 4150 bp, MRNA for KIAA0768 protein) was used to probe the high throughput genome sequence database. This produced “hits” with accession numbers AC078965, AC041038, and AC018649.

Another one of the sequences used was a bovine LPH3 mRNA sequence (accession number AF111094, a 5220 bp, Bos taurus latrophilin 3 splice variant bbbf mRNA) was used to probe the high throughput genome sequence database. This produced “hits” with accession numbers AC016308, AC018649, AC025510, AC026000, and AC041038.

The third sequence used was a rat LPH3 mRNA sequence (accession number AF081157, a 4878 bp, Rattus norvegicus CL3BA MnRNA) was used to probe the high throughput genome sequence database. This produced “hits” with Accession numbers AC025510, AC026000, AC016308, AC018649, and AC041038. This rat LPH3 mRNA was also used to probe the non-redundant database (All non-redundant GenBank CDS translations+PDB+SwissProt+PIR+PRF), and produced “hits” with Accession numbers AC018311, AC07511, and AC020741.

Each of the Accession numbers shown to have high homology to LPH3 represent on a human BAC, most of which are partially sequenced and unordered within the BAC. The following contigs were extracted from the database.

| Accession No. | |
|---|---|
| AC020741 | (complete BAC sequence, 191,804 bp) |
| AC025510 | (13 contigs, 155,633 bp total) |
| AC026000 | (20 contigs, 168,015 bp total) |
| AC016308 | (18 contigs, 143,652 bp total) |
| AC018649 | (12 contigs, 175,923 bp total) |
| AC041038 | (12 contigs, 154,746 bp total) |
| AC078965 | (6 contigs, 166,239 bp total) |
| AC007511 | (complete BAC sequence, 168,822 bp total) |

These sequences were compiled using SeqManlI (part of the commercial DNASTAR DNA analysis package). SeqMan software operates as follows, “SeqMan constructs contigs in a stepwise manner. A newly added sequence is compared to existing contigs using the Martinez algorithm. If a match is found, the newly added sequence is aligned and gaps are inserted using the Needleman-Wunsch algorithm to minimize ambiguity from sequencing errors. If no match is found, the newly added sequence becomes a new contig. SeqMan also checks to see whether a newly added sequence matches any pair of existing contigs, and if so it uses this sequence to bridge the two contigs into one larger contig.” (See, DNASTAR help file).

Also included in the bioinformatic analysis was the partial human CIRL3 cDNA sequence (accession No. AB01831 1) from Nagase et al and the apparent LPH3 splice variant (Lec3, accession no. AF307080) to initially make the contigs. SeqMan results produced five non-overlapping contigs that contained all cDNA sequences present in AB018311 and AF307080, however the human genomic segment that contained the LPH3 olfactomedin-like domain was not present in this assembly.

Primers located in exons for sequencing cDNA (5′ to 3′)

| | | |
|---|---|---|
| Exon 1 | | |
| Exon 1-Forward | GGCCATCGCAGCTACTAAT | SEQ ID NO:56 |
| Exon 1-Reverse | ATTAGTAGCTGCGATGGCC | SEQ ID NO:57 |
| Exon 1.1 | (see below) | |
| Exon 2 | | |
| Exon 2-Forward | GCTCAGATGGAGAATATCCGA | SEQ ID NO:58 |
| Exon 2-Reverse | TCGGATATTCTCCATCTGAGC | SEQ ID NO:59 |
| Exon 3 | | |
| Exon 3-Forward | TGTCCAGGAACCTATAAATAC | SEQ ID NO:60 |
| Exon 3-Reverse | CATGTTGTTGGCATATACCA | SEQ ID NO:61 |
| Exon 3.1 | (see below) | |
| Exon 4 | (see below) | |
| Exon 5 | | |
| Exon 5-Forward | AATTCACCTTGACTCTGAGCT | SEQ ID NO:62 |
| Exon 5-Reverse | AGCTCAGAGTCAAGGTGAATT | SEQ ID NO:63 |
| Exon 6 | | |
| Exon 6-Forward | GAAGAAACCGGAGTACTAGTAC | SEQ ID NO:6 |
| Exon 6-Reverse | GTACTAGTACTCCGGTTTCTTC | SEQ ID NO:65 |
| Exon 7 | | |
| Exon 7-Forward | GTCCAGATCTCAGCAACTGTT | SEQ ID NO:66 |
| Exon 7-Reverse | AACAGTTGCTGAGATCTGGAC | SEQ ID NO:67 |
| Exon 8 | | |
| Exon 8-Forward | AGAAATCACTTGAATGCTG | SEQ ID NO:68 |
| Exon 8-Reverse | CAGCATTCAAGTGATTTCT | SEQ ID NO:69 |
| Exon 8.1 | | |
| Exon 8.1-Forward | CGCTCTTGCAGAGCCTATGT | SEQ ID NO:70 |
| Exon 8.1-Reverse | ACATAGGCTCTGCAAGAGCG | SEQ ID NO:71 |
| Exon 9 | | |
| Exon 9-Forward | GCCACCATGTTGCTTCATA | SEQ ID NO:72 |
| Exon 9-Reverse | TATGAAGCAACATGGTGGC | SEQ ID NO:73 |
| Exon 10 | | |
| Exon 10-Forward | TGGAAGCACTATCCAGCTG | SEQ ID NO:74 |
| Exon 10-Reverse | CAGCTGGATAGTGCTTCCA | SEQ ID NO:75 |
| Exon 11 | | |
| Exon 11-Forward | GTTCAGTAACAAGGTTTATTTG | SEQ ID NO:76 |
| Exon 11-Reverse | CAAATAAACCTTGTTACTGAAC | SEQ ID NO:77 |
| Exon 12 | | |
| Exon 12-Forward | CAATGACAGGTTATTGGTC | SEQ ID NO:78 |
| Exon 12-Reverse | GACCAATAACCTGTCATTG | SEQ ID NO:79 |
| Exon 13 | | |
| Exon 13-Forward | CCAGAGTGACCGTAACACCAT | SEQ ID NO:80 |
| Exon 13-Reverse | ATGGTGTTACGGTCACTCTGG | SEQ ID NO:81 |
| Exon 14 | | |
| Exon 14-Forward | GAGAGTGAACATTCACGTAG | SEQ ID NO:82 |
| Exon 14-Reverse | CTACGTGAATGTTCACTGTC | SEQ ID NO:83 |
| Exon 15 | | |
| Exon 15-Forward | GGAGTTTTATAGGACCAGC | SEQ ID NO:84 |
| Exon 15-Reverse | GCTGGTCCTATAAAACTCC | SEQ ID NO:85 |
| Exon 16 | | |
| Exon 16-Forward | CATACTGCTATACTGAAACCT | SEQ ID NO:86 |
| Exon 16-Reverse | AGGTTTCAGTATAGCAGTATG | SEQ ID NO:87 |
| Exon 16.1 | | |
| Exon 16.1-Forward | GGATAACAGACCCTTCATC | SEQ ID NO:88 |
| Exon 16.1-Reverse | GATGAAGGGTCTGTTATCC | SEQ ID NO:89 |
| Exon 17 | | |
| Exon 17-Forward | GAAAGCACAGTCATCATG | SEQ ID NO:90 |
| Exon 17-Reverse | CTAGATGACTGTGCTTTC | SEQ ID NO:91 |
| Exon 18 | | |
| Exon 18-Forward | GCGAACACATTGCTGTAGT | SEQ ID NO:92 |
| Exon 18-Reverse | ACTACAGCAATGTGTTCGC | SEQ ID NO:93 |
| Exon 19 | | |
| Exon 19-Forward | GACATAAACAGTTCAGCGTCAC | SEQ ID NO:94 |
| Exon 19-Reverse | GTGACGCTGAACTGTTTATGTC | SEQ ID NO:95 |
| Exon 19.1 | | |
| Exon 19.1-Forward | GAGACAAGTATGGGAGTAAA | SEQ ID NO:96 |
| Exon 19.1-Reverse | TTTACTCCCATACTTGTCTC | SEQ ID NO:97 |
| Exon 20 | | |
| Exon 20-Forward | CCTGAGCAACTGTGTGCAAATC | SEQ ID NO:98 |
| Exon 20-Reverse | TTCGGCATCACCACATTTGGC | SEQ ID NO:99 |

Primers located in introns that amplify the corresponding exon from genomic DNA (5′ to 3′)

| | | |
|---|---|---|
| Exon 1 | | |
| Intron 1-Forward | CAGTCTTGGAATACAGAAGAG | SEQ ID NO:100 |
| Intron 1-Reverse | TAAGCCGAGATCGTGCCACCG | SEQ ID NO:101 |
| Exon 1.1 | (see below) | |
| Exon 2 | | |
| Intron 2-Forward | CTGCTTTATATCTTCGTA | SEQ ID NO:102 |
| Intron 2-Reverse | CTTTCCAAATGCTGTTTGC | SEQ ID NO:103 |
| Exon 3 | | |
| Intron 3-Forward | GAGCAAGCTGAATAGATTC | SEQ ID NO:104 |
| Intron 3-Reverse | CAGGGAGGGTCTATGATTCTC | SEQ ID NO:105 |
| Exon 3.1 | (see below) | |
| Exon 4 | (see below) | |
| Exon 5 | | |
| Intron 5-Forward | GGCAGATATGTCTACTCTT | SEQ ID NO:106 |
| Intron 5-Reverse | CATATGTACAACCATCATTGC | SEQ ID NO:107 |
| Exon 6 | | |
| Intron 6-Forward | GAGAGCATTAGGAAGCTTACT | SEQ ID NO:108 |
| Intron 6-Reverse | CTGCAATCAATATCTGATGG | SEQ ID NO:109 |
| Exon 7 | | |
| Intron 7-Forward | GGAGTACTACATTTCTCTAG | SEQ ID NO:110 |
| Intron 7-Reverse | CTTGCTTAATGTATAATGCC | SEQ ID NO:111 |
| Exon 8 | | |
| Intron 8-Forward | GTAAGTTTCCTCAGGAGGA | SEQ ID NO:112 |
| Intron 8-Reverse | GATCCATCCATTCTTCCAC | SEQ ID NO:113 |
| Exon 8.1 | | |
| Intron 8.1-Forward | CCATAGAGTTTTCACTTAGG | SEQ ID NO:114 |
| Intron 8.1-Reverse | CTCAGTCTGTGTTCCTCTTG | SEQ ID NO:115 |
| Exon 9 | | |
| Intron 9-Forward | GGACAATACTGAACTCATGC | SEQ ID NO:116 |
| Intron 9-Reverse | CCTAAGAAAGATAGCACA | SEQ ID NO:117 |
| Exon 10 | | |
| Intron 10-Forward | CAGAAATACTGCAATGGTT | SEQ ID NO:118 |
| Intron 10-Reverse | AATCTGCAAACTCAGTGAG | SEQ ID NO:119 |
| Exon 11 | | |
| Intron 11-Forward | TGAGTATGGAGTGAGATAGG | SEQ ID NO:120 |
| Intron 11-Reverse | CAGGCTGTAATTTAGTTCTCT | SEQ ID NO:121 |
| Exon 12 | | |
| Intron 12-Forward | AGAGAACTAAATTACAGCCTG | SEQ ID NO:122 |
| Intron 12-Reverse | GCCCACTTATGAAACCATTG | SEQ ID NO:123 |
| Exon 13 | | |
| Intron 13-Forward | GATTCACACCTTACGGAAAA | SEQ ID NO:124 |
| Intron 13-Reverse | GCTGCTTATTTATGACAATGGG | SEQ ID NO:125 |
| Exon 14 | | |
| Intron 14-Forward | GCAGATCTATTGTAATGCAG | SEQ ID NO:126 |
| Intron 14-Reverse | CAGCTAGTTCTTCCTTTGAC | SEQ ID NO:127 |
| Exon 15 | | |
| Intron 15-Forward | GGAATGCTTTGCAGCTCACAT | SEQ ID NO:128 |
| Intron 15-Reverse | CAAATAAGTACATGCTTTCCCC | SEQ ID NO:129 |
| Exon 16 | | |
| Intron 16-Forward | CCCAATTCTCGAAACTGGCTT | SEQ ID NO:130 |
| Intron 16-Reverse | TTCAGGCAGATCAAGTCAC | SEQ ID NO:131 |
| Exon 16.1 | | |
| Intron 16.1-Forward | GAGTCTTAGAAGGGATATTGC | SEQ ID NO:132 |
| Intron 16.1-Reverse | TGCTCTTCTGCCTCTACGT | SEQ ID NO:133 |
| Exon 17 | | |
| Intron 17-Forward | GCTTGAAAACTTGTAGTCTC | SEQ ID NO:134 |
| Intron 17-Reverse | TACTATTGGACACTCCCAC | SEQ ID NO:135 |
| Exon 18 | | |
| Intron 18-Forward | GGCTCCAGTTAGGCAAAAT | SEQ ID NO:136 |
| Intron 18-Reverse | TTAGCTCCAATCATAAGGGAC | SEQ ID NO:137 |
| Exon 19 | | |
| Intron 19-Forward | GGTATATAGGCATTTAGGA | SEQ ID NO:138 |
| Intron 19-Reverse | CTTAGCCGACTATACAGCCA | SEQ ID NO:139 |
| Exon 19.1 | | |
| Intron 19.1-Forward | GTGATGATCTTAAGTGTGTA | SEQ ID NO:140 |
| Intron 19.1-Reverse | GACTCCTGATGCATACTGT | SEQ ID NO:141 |
| Exon 20 | | |
| Intron 20-Forward | CATAGTCAATGAATGTTTTTG | SEQ ID NO:142 |
| Intron 20-Reverse | ACAGCATAAAGATTTAGGAGTAC | SEQ ID NO:143 |

Identification of Human LPH3 Exon 1.1

Rat and bovine LPH3 contain exon 1.1 that has no counterpart in the closely related genes LPH1 and LPH2. This exon was not reported in the mRNA in the Lec3 sequence (AF307080). Using the rat and bovine cDNAs as a BLAST probe against our initial LPH3 contig, we found significant homology in the human sequence to both the rat and bovine LPH3 Exon 1.1. This homology occurred at a location that is between Exon 1 and Exon 2 in our initial human LPH3 contig. Primers where designed for sequencing cDNA and genomic DNA to confirm that Exon 1.1 is expressed in human brain and human trabecular meshwork cells.

Primers located in Exon 1.1 for sequencing cDNA

| | | |
|---|---|---|
| Exon 1.1-Forward | TCTTCCACCCAGACATCT | SEQ ID NO:144 |
| Exon 1.1-Reverse | AGATGTCTGGGTGGAAGA | SEQ ID NO:145 |

Primers located in introns that amplify Exon 1.1 from genomic DNA

| | | |
|---|---|---|
| Intron 1.1-Forward | CCAGGAGATGTAATCCTC | SEQ ID NO:146 |
| Intron 1.1-Reverse | CTAAGTGGTAATATCAGCC | SEQ ID NO:147 |

Identification of a Human LPH3 Olfactomedin Domain

In order to obtain the missing olfactomedin domain, a cDNA sequence encoding only the OLF domain from Lec3 (AF307080) was used to search the BAC End Sequence Database available at the Institute for Genomic Research web site (TIGR database). This query produced five overlapping hits. Sequences from Accession numbers AQ900690, AQ419563, AQ440896, AQ333080, and AQ900690 were compiled into a contig using SeqMan as described above. This contig extends slightly beyond the cDNA that codes for the OLF domain of LPH3. Although it appears that there is a gap of unknown distance between the very small mini-exon 3.1 and exon 4 (See FIG. 7x). The BAC end sequences do contain errors, however we were able to construct oligonucleotide primers that allowed us to amplify most of the OLF domain directly from human genomic DNA (Int4F and Int4R). These segments were sequenced and indicate that there are no additional internal introns in the OLF domain

Primers located in Exon 3.1 for sequencing (5′ to 3′)

| | | |
|---|---|---|
| Exon 3.1-Forward | AAGTGGAACAAAAAG | SEQ ID NO:148 |
| Exon 3.1-Reverse | CTTTTTGTTCCACTTC | SEQ ID NO:149 |

Primers located in introns that amplify Exon 4 from genomic DNA (5′ to 3′)

| | | |
|---|---|---|
| Intron 4-Forward | GCATTCCATTTGGTGAAAAAT | SEQ ID NO:1 |
| Intron 4-Reverse | TGAAAGTGATCACAGCAAACC | SEQ ID NO:151 |

Primers located in Exon 4 for sequencing (5′ to 3′)

| | | |
|---|---|---|
| Exon 4.0-Forward | GGAGTATACCAGAGTGAACATTTG | SEQ ID |
| | | NO:152 |
| Exon 4.0-Reverse | CAAATGTTCACTCTGGTATACTCC | SEQ ID |
| | | NO:153 |
| Exon 4.1-Forward | TCCAAGGATGACTTCATTGCTG | SEQ ID |
| | | NO:154 |
| Exon 4.1-Reverse | CAGCAATGAAGTCATCCTTGGA | SEQ ID |
| | | NO:155 |
| Exon 4.2-Forward | GAGAGGCTATCATAGCAAATGC | SEQ ID |
| | | NO:156 |
| Exon 4.2-Reverse | GCATTTGCTATGATAGCCTCTC | SEQ ID |
| | | NO:157 |
| Exon 4.3-Forward | GACCAAAGCAAGGATAGTTTGG | SEQ ID |
| | | NO:158 |
| Exon 4.3-Reverse | CCAAACTATCCTTGCTTTGGTC | SEQ ID |
| | | NO:159 |

Assembly of the complete human LPH3 Sequence

The final product coding sequence for LPH3 is an assembly of six contigs containing approximately 675,053 base pairs with five gaps present in intronic sequences. This sequence is presented in FIG. 7 as SEQ ID NO:5. The coding sequence (from the 5′ ATG start codon to the 3′ TAG stop codon) is contained within approximately 407,422 base pairs. The sequence is marked with the positions of the exons so that the gene structure is evident (See, FIG. 7). The final sequence that was assembled contains all of the information needed to design primers that flank the exons and amplify the exons and splice sites for purposes of, for example, mutation screening or preparation of constructs that would be used in expression vectors to produce portions of the protein.

### Example 2

### Human Trabecular Meshwork Cells Express LPH3

This example describes experiments that demonstrate that the LPH3 peptide is expressed in human Trabecular Meshwork (HTM) cells. HTM that were cells derived from non-glaucomatous eyes were grown in Dulbecco's modified Eagle's medium with 15% fetal calf serum with transforming growth factor-pl (TGFp1) at 37° C. under 10% CO₂. After achieving confluency, HTM cells were induced using 500 nM dexamethasone for three weeks.

RNA was then prepared using a one-step isolation using TRIzol reagent (Life Technologies). One (1) ml of TRIzol was added to each of two flasks to solubilize the cells. The suspension was scraped into a tube and incubated for 5 minutes at room temperature. Next, 0.4 ml of chloroform was added with vigorous mixing followed by centrifugation at 12,000 g for 15 minutes. The aqueous phase containing RNA was transferred to a new tube and 0.5 ml isopropanol was added for 10 minutes at room temperature. An RNA pellet was obtained after centrifugation at 12,000 g for 15 minutes. The pellet was washed twice with 1 ml 75% ethanol and resusupended in 20 μl of nuclease-free water.

Next, the RNA was used as substrate material to generate cDNA templates. Two (2) micrograms of human trabecular meshwork RNA was adjusted to a total volume of 13.5 pl and denatured for 5 minutes at 65° C., and placed on ice. Then 5 μl of 5x 1st strand buffer was added (final concentration: 50 mM Tris-HCl pH 8.3, 75 mM KCl, 3 mM MgCl₂), 1 μl of 0.1 M DTT was added (final concentration of 4 mM), 1 μl of random hexamers diluted 1:6 with water, 1 μl of a mixture of 25 mM dNTP solution [dATP, dCTP, dGTP, dTTP (2′-deoxynucleoside 5′-triphosphates), final concentration of 1 mM], and 0.5 μl of RNase inhibitor was added to the mixture. These components were incubated for 10 minutes at room temperature. Then 3 μl (600 units) of M-MLV (Moloney Murine Leukemia Virus) Reverse Transcriptase was added and incubated at 37° C. for 90 minutes. The reaction was stopped by heating to 95° C. for 10 minutes and the cDNA samples stored at −20C.

Next, exon primers were designed that would span intronic sequences of human LPH3 (all shown 5′ to 3′ direction):

| Exons included | Primers | Seuence | SEQ ID NO: |
|---|---|---|---|
| (1-3) | 1F | GGCCATCGCAGCTACTAAT | SEQ ID NO:56 |
| | 3R | GATTTTATAGGTTCCTGGACA | SEQ ID NO:160 |
| (1.1-3) | 1.1F | TCTTCCACCCAgACATCT | SEQ ID NO:144 |
| | 3R | GATTTTATAGGTTCCTGGACA | SEQ ID NO:160 |
| (2-5) | 2F | gCTCAgATggAgAATATCCgA | SEQ ID NO:58 |
| | 5R | AGCTCAGAGTCAAGGTGAATT | SEQ ID NO:63 |
| (3-5) | 3F | TGTCCAGGAACCTATAAATAC | SEQ ID NO:60 |
| | 5R | AGCTCAGAGTCAAGGTGAATT | SEQ ID NO:63 |
| (5-8) | 5F | AATTCACCTTGACTCTGAGCT | SEQ ID NO:62 |
| | 8R | CAGCATTCAAGTGATTTCT | SEQ ID NO:69 |
| (5-8.1) | 5F | AATTCACCTTGACTCTGAGCT | SEQ ID NO:62 |
| | 8.1R | ACATAggCTCTgCAAgAgCg | SEQ ID NO:71 |
| (8-13) | 8F | AGAAATCACTTGAATGCTG | SEQ ID NO:68 |
| | 13R | ATGGTGTTACGGTCACTCTGG | SEQ ID NO:81 |
| (9-12) | 9F | GCCACCATGTTGCTTCATA | SEQ ID NO:72 |
| | 12R | GACCAATAACCTGTCATTG | SEQ ID NO:79 |
| (12-16) | 12F | CAATGACAGGTTATTGGTC | SEQ ID NO:78 |
| | 16R | AGGTTTCAGTATAGCAGTATG | SEQ ID NO:87 |
| (14-17) | 14F | GAGAGTGAACATTCACGTAG | SEQ ID NO:82 |
| | 17R | CTAGATGACTGTGCTTTC | SEQ ID NO:91 |
| (16.1-17) | 16.1F | GGATAACAGACCCTTCATC | SEQ ID NO:88 |
| | 17R | CTAGATGACTGTGCTTTC | SEQ ID NO:91 |
| (16-20) | 16F | CATACTGCTATACTGAAACCT | SEQ ID NO:86 |
| | 20R | TTCggCATCACCACATTTggC | SEQ ID NO:99 |
| (17-19) | 17F | GAAAGCACAGTCATCATG | SEQ ID NO:90 |
| | 19R | AAAGGAAGACTCTGACTGC | SEQ ID NO:161 |
| (19-20) | 19F | gACATAAACAgTTCAgCgTCAC | SEQ ID NO:94 |
| | 20R | TTCggCATCACCACATTTggC | SEQ ID NO:99 |
| (19.1-LEC3-3′) | 19.1 F | GAGACAAGTATGGGAAGTA | SEQ ID NO:162 |
| | lec3-3′ | GGTAGAGTATCCATGACAC | SEQ ID NO:163 |
| (19.1-20) | 19.1F | GAGACAAGTATGGGAAGTA | SEQ ID NO:162 |
| | 20R | TTCggCATCACCACATTTggC | SEQ ID NO:99 |

PCR reactions (25 μl final volume) were prepared using AmpliTaq gold (Applied Biosystems) as follows: Final concentrations: 15 mM Tris-HCl pH 8.0, 50 mM KCl, 2 mM MgCl₂, [DATP, dCTP, dGTP, dTTP (2mM each)], 0.4 micromolar of appropriate forward (F) and reverse (R) primer, and 2.5 units of AmpliTaq gold polymerase. This mixture was added to 1 μl of cDNA prepared from the HTM cell RNA and was then subjected to PCR using a PTC-100 thermocycler. After an initial denaturation step of 95° C. for 10 minutes, 35 cycles of 95° C. for 30 seconds, 57° C. for 45 seconds, 72° C. for 60 seconds were followed by a final extension step at 72° C. for 10 minutes and held at 4° C. Samples were electrophoresed on TAE agarose gels to determine the size of the amplified exons visualized using ethidium staining and ultraviolet light. Successful amplification resulted with all of the primers using the prepared cDNA, except no PCR products could be detected utilizing primers 16.1F or 19.1F, indicating these exons probably are not expressed in HTM cells.

### Example 3

### Screening Patient Samples for Mutations in LPH Genes

This example demonstrates one general method for looking for glaucoma related mutations in LPH genes in patient samples. In this example, the LPH3 and LPH1 genes from patient samples were analyzed.

Blood samples were obtained from glaucoma patients recruited through the Kellog Eye Center Glaucoma Genetics program (Ann Arbor, Mich.). The patient population consisted of 42 unrelated individuals with primary open-angle glaucoma (age of onset at 35 years and older). The ethnicity of this group included: 9 African-Americans, 1 Hispanic, 31 Caucasians, and 1 unknown. The patient population also included 15 individuals with juvenile onset open-angle glaucoma (age of onset is prior to age 35). The ethnicity of this group included 2 African-Americans, 2 Hispanics, and 11 Caucasians. One (1) individual who did not have glaucoma was used as a sequencing control. Note, due to variations in experimental procedures, not every individual was necessarily assayed for each variation in this example.

Genomic DNA was isolated from whole blood of each of the patient samples using a Puregene DNA Isolation Kit (Gentra Systems) according to the manufacturer's protocol. Polymerase Chain Reaction of LPH1 and LPH3 gene fragments were amplified from genomic DNA using an AmpliTaq Gold PCR kit. PCR reactions (25 μl final volume) were prepared using AmpliTaq gold polymerase (Applied Biosystems) as follows: Final concentrations: 15 mM Tris-HCl pH 8.0, 50 mM KCl, 2 mM MgCl₂, [DATP, dCTP, dGTP, dTTP (0.2mM each)], 0.4 micromolar of appropriate forward and reverse primer (see primer pairs below), and 2.5 units of AmpliTaq gold polymerase. This mixture was added to 50 ng of genomic DNA and was then subjected to PCR using a PTC-100 thermocycler. After an initial denaturation step of 95° C. for 10 minutes, 35 cycles of 95° C. for 30 seconds, 57° C. for 45 seconds, 72° C. for 60 seconds were followed by a final extension step at 72° C. for 10 minutes and held at 4° C. Samples were electrophoresed on TAE agarose gels to determine the size of the amplified products using ethidium staining and ultraviolet light. The fragments were purified and used as templates for DNA sequence determination.

PCR-amplified genomic fragments were first purified using Qiagen Qiaquick purification spin columns. One hundered (100) ng of purified PCR product was sequenced using a ABI Prism Big Dye Terminator Cycle Sequencing Ready Reaction kit (Perkin Elmer) and 1.6 picomole of the appropriate primer described below. After a first step denaturation at 95° C. for 5 minutes, 25 cycles were carried out at 95 ° C. for 30 seconds, 52° C. for 10 seconds, 60° C. for 4 minutes. Samples were adjusted to 65% total isopropanol and precipitated at room temperature for 15 minutes, then centrifuged at 2000 x g for 45 minutes. Samples were resuspended in 4 μl of a 1:5 solution of Blue Dextran:deionized formamide. Samples were size fractionated by electrophoresis on a 7.5% denaturing polyacrylamide sequencing gel and analyzed on an ABI377 Semi-automated Sequencer.

Mutation Screening of LPH1-Primers Used (all primers shown in 5′ to 3′ direction)

| Exon | Mutation | Name | Sequence | SEQ ID NO: |
|---|---|---|---|---|
| 4 | Arg329Arg | 875F | ACACACGAGTCAGAGCACCAG | SEQ ID NO:164 |
| | CGT to CGC | 1480R | GCGGTTGGCATTGGTGTTGAA | SEQ ID NO:165 |
| 11 | Glu750Asp | 6F | GTTTAGAACTGCCTTCAGCTGAG | SEQ ID NO:166 |
| | GAA to GAC | 6R | TACTGCCCGTGATCTCTCTAGG | SEQ ID NO:167 |
| 12 | Tyr815Tyr | 6F | GTTTAGAACTGCCTTCAGCTGAG | SEQ ID NO:166 |
| | TAC to TAT | 6R | TACTGCCCGTGATCTCTCTAGG | SEQ ID NO:167 |

Results for the Arg329Arg polymorphism were as follows. Of 30 POAG individuals screened, 8 out of 30 individuals were heterozygous for this polymorphism, 1 out of 30 individuals were homozygous for the polymorphism. Since this is not a mutation that alters the amino acid sequence of the protein and does not segregate with the disease phenotype, further testing of this polymorphism was not undertaken. This polymorphism is recorded as the “normal” sequence by Nagase et aL (accession No. AB020628).

Results for the Glu75OAsp polymorphism were as follows. This polymorphism was found in 4 out of 74 POAG probands, and in 3 out of 18 normals. Since this mutation does not segregate with the disease phenotype, no further screening was performed.

Results for the Tyr815Tyr polymorphism were as follows. Thirteen (13) of 71 POAG probands were heterozygous, and 1 of 8 normals were homozygous for this polymorphism. Since this is not a mutation that alters the amino acid sequence of the protein and does not segregate with the disease phenotype, no further screening for this polymorphism was undertaken. This polymorphism is recorded as the “normal” sequence by Nagase et al (accession NO. AB020628).

Mutation Screening of LPH3-Primers Used (all primers shown in 5′ to 3′ direction)

| Exon | Mutation | Name | Sequence | SEQ ID NO: |
|---|---|---|---|---|
| 1.1 | Leu33Met | Int1.1F | CCAGGAGATGTAATCCTC | SEQ ID NO:168 |
| | TTG to ATG | Int1.1R | CTAAGTGGTAATATCAGCC | SEQ ID NO:169 |
| 4 | His272His | Ex4F | GGAGTATACCAGAGTGAACATTTG | SEQ ID NO:152 |
| | CAC to CAT | Ex4R | CAAATGTTCACTCTGGTATACTCC | SEQ ID NO:153 |
| 4 | Leu360Ile | Ex4F | GGAGTATACCAGAGTGAACATTTG | SEQ ID NO:152 |
| | CTA to ATA | Ex4R | CAAATGTTCACTCTGGTATACTCC | SEQ ID NO:153 |
| 4 | Phe382Leu | Ex4F | GGAGTATACCAGAGTGAACATTTG | SEQ ID NO:152 |
| | TTT to CTT | Ex4R | CAAATGTTCACTCTGGTATACTCC | SEQ ID NO:153 |
| 20 | ASN1298Ile | Int20F | CATAGTCAATGAATGTTTTTG | SEQ ID NO:142 |
| | AAC to ATC | Ex20R | TTCGGCATCACCACATTTGGC | SEQ ID NO:99 |

The results of this screening could be analyzed as above for the LPH1 tests to determine if these polymorphisms co-segregate with the disease phenotype.

### Example 4

### Screening LPH3 for Leu33Met Mutation

This example describes screening patient samples for a Leu33Met mutation in the LPH3 sequence by allele specific hybridization. Two 19 base oligonucleotides are designed with the polymorphism centered at position 10 [Control(Leu): 5′-GCTGCTCCAtTGCGACACG (SEQ ID NO:170), and Mutant(Met):5′-GCTGCTCCA-a-TGCGACACG (SEQ ID NO:171]. Screening is performed on members of each proband's family and a test panel consisting of 100 normal individuals of ages 35 to 83 years (mean 55 years), 50 unrelated JG individuals with POAG diagnosed prior to age 35, and 100 individuals with POAG diagnosed after age 35. Genomic DNA is amplified by PCR using primers Intl.1F (5′-CCAGGAGATGTAATCCTC, SEQ ID NO:168) and Intl.1R (5′-CTAAGTGGTAATATCAGCC, SEQ ID NO:169) and the 520 bp product is purified from TAE agarose gels. PCR-amplified DNA is bound to Hybond N+ (Amersham) membrane under alkaline denaturing conditions and fixed to the membrane using a Stratalinker UV lightsource (Stratagene, La Jolla, Calif.). Oligonucleotides are end-labeled by transfer of ³²p from gamma- ³²P dATP (Amersham) using T4 polynucleotide kinase (New England Biolabs, Beverly, Mass.). Blots are prehybridized for 60 min at 47° C. in 5 x SSPE pH7.4 (0.75 M NaCl, 50 mM NaH₂PO₄, 5 mM EDTA), 5 x DET (0.1% polyvinylpyrrolidone, 0.1% ficoll, 0.1% BSA, 1 mM Tris-HCl pH8.0, 1 mM EDTA), 0.5% sodium dodecyl sulfate (SDS) with 100 μg/ml salmon sperm DNA. Following prehybridization, the ³²P-end labeled mutant oligonucleotide is added for 60 min at 47° C. with gentle agitation. Non-specific label is removed by sequential 10 minute washes in: 2 x SSPE, 0.1% SDS at 25° C.; 5 x SSPE, 0.1% SDS at 52° C.; and 2 x SSPE, 0.1% SDS at 58° C.. The blot is briefly air-dried and exposed to film, after which the blot is stripped using 0.4 N NaOH at 42° C. for 30 minutes, neutralized with 0.1 x SSC (15 mM NaCl, 16.5 mM NaCitrate), 0.1% SDS, 0.2 M Tris-HCl pH7.5 at 42° C. for 30 minutes, and rehybridized with the normal oligonucleotide under the same conditions. The exposed film is evaluated for the relative signal intensity for each probe based on comparison to included negative, positive, and no DNA controls either by eye or quantitated using densitometer.

Homozygotes who do not have the mutation show strong signals for the control oligo and background signal for the mutant oligo. Homozygotes who have the mutation show strong signals for the mutant oligo and background signal for the control oligo. Heterozygotes for the mutation show medium signal intensity for both mutant and control oligo.

### Example 5

### Localization of LPH3 to a Glaucoma Inclusion Interval on Chromosome 4

This Example describes the localization of the human LPH3 gene to a glaucoma inclusion interval on chromosome 4 that was previously reported by Wiggs et al., in an article entitled “Genome-wide scan for adult onset primary open angle glaucoma”, Hum. Mol. Genet. 9:1109-1117, (2000). The partial human sequence for LPH3 (Accession number AB018311 for KIAA0768 protein) was queried against the “Unigene” database (See, NCBI web page, UniGene Resource, hereinafter “UniGene Web Resource”) which provided a UniGene locus:Hs.21917. This UniGene locus provided mapping information to chromosome 4 via a UniSTS entry:stSG4795. The reference interval for stSG4795 was D4S1619-D4S392 (see reference) on the GM99-GB4 Map.

The relationship between the genetic markers and the double-peaked glaucoma inclusion interval on chromosome 4 reported in FIG. 1 by Wiggs et al is as follows: D4S2366 - low; D4S403 - start of peak 1; D4S2639 - high on peak 1; D4S2397 -highest on peak 1; D4S2632 - slope on peak 1; D4S1627 - “valley” between peak 1 and peak 2; D4S3248 - high on peak 2; D4S2367 - high on peak 2; and D4S400 - low.

Unfortunately, these markers did not directly correspond to the same set of markers used to map LPH3. As such, the location of the markers that flank LPH3 relative to those reported by Wiggs et al. had to be calculated. Using the start of the first peak as a baseline, the distance between D4S403 (Wiggs) and D4S398 (LPH3) was calculated to be 48.6 centimorgans. Calculating from the end of the second peak D4S1541 (LPH3) and D4S400 (Wiggs) gives a distance of 14.3 centimorgans. The total distance over both peaks of Wiggs inclusion interval is 62.9 cM. The ratio between D4S403 and one end of the smaller interval that flanks LPH3 is (48.6/62.9)=0.7727. When this is applied to Wiggs FIG. 1, LPH3 is located near the top of peak 2. Likewise, from the other direction (D4S400) 14.3/62.9=0.2273, which also places LPH3 at the top of peak 2.

Since this initial finding, additional work has located LPH3 between two markers, D4S3248 and D4S3248, near the top of peak 2. D4S3248 maps directly to chromosome 4 at position 62,975,706 bp on the Oct. 7, 2001 Freeze. The partial LPH3 sequence (AB08311) maps to chromosome 4 at 65,869,006 bp - 66,030,274 bp. The flanking marker on peak 2 (D4S2367) still does not map directly, however the corresponding DNA was extracted from Accession number G08338. This 306 bp segment contains the STS D4S3267 sequence. When the Oct 7, 2001 Freeze was searched for this segment using BLAT (available at the NCBI web page), it was found to be located at 71,402,479 bp - 71,402,772 bp on chromosome 4.

Interpretation of this physical data allows a more precise localization of LPH3 to the glaucoma inclusion interval. D4S2366 - low; D4S403 - start of peak 1; D4S2639 - high on peak 1; D4S2397 - highest on peak 1; D4S2632 - slope on peak 1; D4S 1627 - “valley” between peak 1 and peak 2; D4S3248 - high on peak 2; (2,890,000 bp) LPH3 (5,370,000 bp); D4S2367 - high on peak 2; D4S400 - low. Listed below is the output generated by the UniGene Web Resource:

UniGene Cluster Hs.21917 KIAA0768 (partial CIRL3)

KIAA0768 protein

SEE ALSO

LocusLink: 23284

HomoloGene:Hs.21917

Selected Model Organism Protein Similarities organism, protein, percent identity/length of aligned region H. sapiens: pir:137225-137225 leucocyte antigen CD97 39%/391 aa

M. musculus: sp:Q61549-EMR1 MOUSE CELL SURFACE 32%/532 aa

GLYCOPROTEIN EMR1 PRECURSOR

R. norvegicus: pir:T17187-T17187 CL3AB protein-rat 97%/1239 aa

C. elegans: pir:T15308-T15308hypotheticalprotein B0286.2-34%/663 aa Caenorhabditis elegans

Mapping Information Chromosome: 4

UniSTS entries: RH82189

UniSTS entries: stSG4795 (partial CIRL3)

Expression Information cDNA sources: Adrenal gland, Brain, CNS, Ear, Eye, Heart, Kidney, Lung, Ovary, Placenta, Pooled, Prostate, Testis, adrenal gland, brain, nervous_normal, nervous_tumor, placenta_normal, testis_normal

Sage: Gene to Tag mapping mRNA/Gene Sequences (3)

AB018311 Homo sapiens mRNA for KIAA0768 protein, partial cds

AF307080 Homo sapiens lectomedin-3 (LEC3) mRNA, complete cds

AK000781 Homo sapiens cDNA FLJ20774 fis, clone COL06031

Est Sequences (10 of 92)[Show all ESTs]

| | | | |
|---|---|---|---|
| R44742 | cDNA clone IMAGE: 33829 | Brain | 3′ read 3.9 kb |
| H29414 | cDNA clone IMAGE: 52627 | Brain | 5′ read 3.8 kb |
| H29321 | cDNA clone IMAGE: 52627 | Brain | 3′ read 3.8 kb |
| H15996 | cDNA clone IMAGE: 48681 | Brain | 5′ read 2.5 kb |
| AW024611 | cDNA clone IMAGE: 2526146 | Kidney | 3′ read 2.2 kb |
| R17192 | cDNA clone IMAGE: 32029 | Brain | 5′ read 1.9 kb |
| R41743 | cDNA clone IMAGE: 32029 | Brain | 3′ read 1.9 kb |
| H06374 | cDNA clone IMAGE: 44197 | Brain | 5′ read 1.9 kb |
| H06375 | cDNA clone IMAGE: 44197 | Brain | 3′ read 1.9 kb |
| AA234811 | cDNA clone IMAGE: 669551 | | 5′ read 1.9 kb |

Homo sapiens mRNA for KIAA0768 protein, partial cds

Cross-References

UniGene Hs.21917 Homo sapiens mRNA for KIAA0768 protein, partial cds

RH Mapping Results

| | | |
|---|---|---|
| stSG4795 | GB4 Map: | Chr.4 |
| | Reference interval: | D4S1619-D4S392 (67.7-77.9 cM) |
| | Physical position: | 371.17 cR3000 (P0.03) |
| | RH details: | PHdb RPH15997 |
| | Typed by: | Sanger Centre |

Electronic PCR Results

mRNAs (from GenBank PRI division)

AB018311.1 Homo sapiens mRNA for KIAA0768 protein, partial cds STS 3100 . . . 3265 bp: stSG4795

ESTs (from GenBank EST division)

AA236896 zs43fl2.s1 Soares NhHMPu S1 Homo sapiens cDNA clone 687983 3′ STS 90 . . . 255 bp: stSG4795

AI266046 qq12a12.x1 Soares_NhHMPu_S1 Homo sapiens cDNA clone

IMAGE:1932286 3′, mRNA sequence [Homo sapiens] STS 87 . . . 252 bp: stSG4795

AI378341 tc52d11.x1 Soares_NhHMPu_S1 Homo sapiens cDNA clone

IMAGE:2068245 3′, mRNA sequence [Homo sapiens] STS 104 . . . 269 bp: stSG4795

H05071 y169g04.s1 Homo sapiens cDNA clone 43403 3′.

STS 95 . . . 260 bp: stSG4795

H06375 y179b12.s1 Homo sapiens cDNA clone 44197 3′.

STS 96 . . . 261 bp: stSG4795

H23096 ym51d10.s1 Homo sapiens cDNA clone 52061 3′.

STS 95 . . . 260 bp: stSG4795

R61356 yh15b07.sl Homo sapiens cDNA clone 37765 3′.

STS 95 . . . 260 bp: stSG4795

Z39855 H. sapiens partial cDNA sequence; clone c-1kh11.

STS 86 . . . 251 bp: stSG4795

Unfinished genomic (from GenBank HTG division)

AC0075 11.1 Homo sapiens clone hRPK.84_A_{—}1, WORKING DRAFT SEQUENCE,

8 unordered pieces

STS 106258 . . . 106424bp: stSG4795

Chromosome 4: D4S1619-D4S392

The interval shown is on the GB4 map

See also: equivalent interval on G3 map

About This Interval

Top of interval: D4S1619 (67.7 cM)

Bottom of interval: D4S392 (77.9 cM)

Genetic size of bin: 10 cM

Physical size of bin: 73 cR3000

Next interval up

| | | | | | |
|---|---|---|---|---|---|
| 67.7 | 339.03 | F | AFM356tc5 | D4S1619 | Microsatellite |
| | | | | | anchor AFM356tc5 |
| | 344.38 | | P0.25 | p4-2187 | Unknown |
| | 344.77 | | P0.22 | stSG32052 | EST |
| | 344.79 | | P0.25 | sts-T87883 | EST |
| | 344.88 | | P1.46 | R43610 | ESTs |
| | 345.43 | | P0.47 | sts-N30696 | EST |
| | 345.43 | | P1.64 | stSG28140 | ESTs |
| | 345.52 | | P1.23 | X76029 | H. sapiens mRNA |
| | | | | | for neuromedin U |
| | 345.85 | | P1.83 | stSG3810 | ESTs, Weakly |
| | | | | | similar |
| | 345.96 | | P>3.00 | stSG49343 | EST |
| | 347.35 | | P>3.00 | stSG36080 | EST |
| | 347.80 | | P>3.00 | sts-N21336 | ESTs |
| | 349.44 | | P1.47 | R80639 | ESTs |
| | 349.55 | | P>3.00 | stSG29793 | KIAA0635 |
| | | | | | KIAA0635 gene |
| | 350.01 | | P0.35 | WIAF-931 | Human neural- |
| | | | | | restrictive |
| | 350.24 | | P>3.00 | stSG52113 | ESTs |
| 68.4 | 350.66 | | P0.26 | D4S1592 | Microsatellite |
| | | | | | marker AFM292xe1 |
| | 350.66 | | P>3.00 | WI-22710 | ESTs |
| | 350.71 | | P2.16 | WI-20631 | PPAT |
| | 350.90 | | P0.26 | CM4.1450 | Unknown |
| | 350.97 | | P>3.00 | stSG35912 | EST |
| | 351.02 | | P>3.00 | F13619 | ESTs |
| | 351.02 | | P1.84 | U22314 | REST RE1- |
| | | | | | silencing |
| | | | | | transcription factor |
| | 351.02 | | P>3.00 | WI-11762 | ESTs |
| | 351.25 | | P>3.00 | stSG13150 | ADE2H1 mRNA |
| | 351.25 | | P>3.00 | stSG28775 | EST |
| | 352.01 | | P0.85 | A006J04 | ESTs |
| | 352.82 | | P>3.00 | sts-N32858 | ESTs |
| | 352.94 | | P>3.00 | N35889 | FRG1 |
| | | | | | FSHD region gene 1 |
| | 353.05 | | P1.31 | R69607 | ESTs |
| | 353.17 | | P>3.00 | stSG58226 | ESTs |
| | 353.40 | | P0.87 | sts-H84834 | ESTs |
| | 353.74 | | P0.33 | H49911 | ESTs |
| | 353.86 | | P2.32 | sts-AA005396 | ESTs |
| | 354.80 | | P>3.00 | stSG62407 | EST |
| | 355.61 | | P>3.00 | stSG41374 | ESTs |
| | 356.54 | | P>3.00 | stSG12546 | EST |
| | 358.88 | | P>3.00 | WI-14844 | ESTs |
| | 360.64 | | P>3.00 | SGC44241 | ESTs |
| | 362.83 | | P2.15 | stSG4355 | ESTs |
| | 363.44 | | P1.49 | sts-T16787 | ESTs |
| | 363.44 | | P1.49 | NIB1853 | ESTs |
| | 364.63 | | P1.00 | WI-21794 | ESTs |
| | 364.72 | | P0.98 | 1753 | EST |
| | 364.94 | | P0.06 | A004G27 | ESTs |
| | 367.63 | | P0.01 | stSG4082 | ESTs |
| 71.5 | 369.55 | F | | D4S398 | Microsatellite |
| | | | | | marker AFM135xc3 |
| | 371.17 | | P0.03 | stSG4795 | KIAA0768 protein |
| | | | | | (partial CIRL3) |
| | 378.76 | | P1.78 | sts-T03224 | ESTs |
| | 378.76 | | P>3.00 | A007C18 | dipeptidyl |
| | | | | | peptidase . . . |
| | 378.76 | | P1.78 | FB5A10 | ESTs |
| | 380.20 | | P>3.00 | stSG62504 | EST |
| | 380.42 | | P>3.00 | 1085 | EST |
| | 381.08 | | P1.84 | stSG16475 | EST |
| | 381.63 | | P0.13 | stSG15781 | ESTs |
| | 384.47 | | P1.22 | L36644 | EPHA5 EphA5 |
| | 389.31 | | P0.00 | sts-N22219 | EST |
| | 389.41 | | P0.01 | sts-N25770 | EST |
| 74.1 | 389.51 | F | | D4S1541 | Microsatellite |
| | | | | | marker AFM036yb2 |
| | 400.39 | | P1.80 | stSG48522 | EST |
| | 400.56 | | P1.47 | T92782 | ESTs |
| 77.3 | 400.67 | | P1.28 | D4S409 | Microsatellite |
| | | | | | marker AFM183xd6 |
| | 401.07 | | P1.83 | L03380 | GNRHR |
| | | | | | gonadotropin- |
| | | | | | releasing |
| | 401.07 | | P1.83 | L03380 | GNRHR |
| | | | | | gonadotropin- |
| | | | | | releasing |
| | 401.86 | | P1.18 | A005E33 | ESTs |
| | 401.86 | | P1.34 | WI-12217 | DDX16 DEAD/H |
| | 402.08 | | P0.97 | stSG22537 | ESTs |
| | 402.08 | | P1.23 | EST160613 | Human Ig J chain |
| | | | | | gene |
| | 402.08 | | P1.54 | H55755 | H. sapiens |
| | | | | | centromere |
| | 402.75 | | P1.53 | H53013 | ESTs |
| | 403.41 | | P0.15 | T79182 | EST |
| | 403.63 | | P1.13 | A006E40 | Human Ig J chain |
| | | | | | gene |
| | 403.92 | | P1.18 | stSG53400 | ESTs |
| | 404.03 | | P2.48 | M12759 | Unknown |
| | 404.14 | | P1.23 | stSG47209 | ESTs |
| | 404.25 | | P1.09 | U07231 | GRSF1 G-rich RNA |
| | | | | | sequence binding |
| | 407.22 | | P1.01 | S83198 | BPLP = basic |
| | | | | | proline-rich |
| | 407.76 | | P0.95 | D89501 | Human PBI gene, |
| | | | | | complete cds |
| | 408.17 | | P0.82 | U06641 | UGT2B15 UDP |
| | | | | | glycosyltransferase |
| | | | | | 2 |
| | 409.00 | | P0.00 | L10615 | Unknown |
| | 411.14 | | P0.00 | T67747 | UGT2B7 UDP |
| | | | | | glycosyltransferase |
| | | | | | 2 |
| | 411.84 | | P0.00 | stSG45282 | CSN1 casein, alpha |
| 77.9 | 411.94 | F | | D4S392 | Microsatellite |
| | | | | | anchor AFM022xc1 |

### Example 6

### Detecting Interactions Between LPH and TIGR

This Example describes certain methods for detecting interactions between LPH and TIGR. To test for binding/interaction of TIGR and LPH, the human TIGR gene and LPH3 gene, or parts of those genes, are cloned into a ‘bait vector’ such as pHybLex/Zeo and a ‘prey’ vector such as pYESTrp2 of a yeast two-hybrid assay system such as the Hybrid Hunter yeast two-hybrid system (Invitrogen, carlsbad, Calif.). The two vector-insert constructs are transformed into one of several yeast strains (L40, EGY48, EGY191) in which the LexA operator has been placed into the 5′ flanking region of the reporter gene. TIGR-LPH3 interaction is detected when the bait and the prey interact, causing transcription of the reporter gene and reporter gene activity. Selection for retention of vectors involves, for example, the use of Zeocin. Reporter genes include but are not limited to HIS3, LacZ, or GAL4. Alternatively other vector systems or yeast strains engineered for use in yeast-two hybrid assays could be used. Subsequent assays to provide additional information on binding using gel technology to detect protein fusions or monitor binding employs antibodies available from Invitrogen or other sources, including Anti-LexA antibody and the anti-V5 antibody as well as other antibodies against TIGR or LPH3.

Alternatively, TIGR-LPH3 interactions can be studied by labelling the recombinant TIGR. By the appropriate choice of expression vector, a poly-Histidine (6xHIS) or FLAG epitope can be incorporated into either the carboxy or amino terminal end of TIGR. Such epitope-tagged protein is readily purified over an affinity column appropriate to the epitope tag. Labelled TIGR is incubated with either cells that express LPH3 (endogenous or via transfection of a cloned LPH3 gene) or with purified membranes from cells known to express LPH3. TIGR used in such binding assays may or may not be radiolabelled using ³⁵S-Methionine. After several washing steps in cell culture media (for cells) or cell membrane resuspension buffer (for membranes), cells or membranes are solubilized and the fractions examined by immunoblots. Competition assays using TIGR with either a different or no epitope tag would be expected to reduce any TIGR-LPH3 interactions. Alternatively, membrane proteins can be bound to filters and retained radiolabelled TIGR can be assayed by counting bound ³⁵S. Variation of binding conditions (time course, temperature, etc) can also be performed.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodinents, it should be understood that the invention as claimed should not be unduly limited to such specific embodiments. Indeed, various modifications of the described modes for carrying out the invention which are obvious to those skilled in chemistry, and molecular biology or related fields are intended to be within the scope of the following claims.

We claim:

1. A composition comprising an isolated nucleic acid, wherein said nucleic acid comprises a sequence that shares at least 96% identity with SEQ ID NO:1 or the complement of SEQ ID NO:1.

2. The composition of claim 1, wherein said sequence shares at least 97% identity with SEQ ID NO:1 or the complement of SEQ ID NO:1.

3. The composition of claim 1, wherein said sequence shares at least 98% identity with SEQ ID NO:1 or the complement of SEQ ID NO:1.

4. The composition of claim 1, wherein said sequence is operably linked to a heterologous promoter.

5. The composition of claim 1, wherein said sequence is contained within a vector.

6. A composition comprising an isolated peptide encoded by the sequence of claim 1.

7. A composition comprising an isolated nucleic acid, wherein said nucleic acid comprises a sequence encoding a peptide that shares at least 96% identity with SEQ ID NO:3.

8. The composition of claim 7, wherein said peptide shares at least 97% identity with SEQ ID NO:3.

9. The composition of claim 7, wherein said peptide shares at least 98% identity with SEQ ID NO:3.

10. The composition of claim 7, wherein said sequence is operably linked to a heterologous promoter.

11. The composition of claim 7, wherein said sequence is contained within a vector.

12. A composition comprising an isolated peptide encoded by the sequence of claim 7.

13. A composition comprising an isolated peptide, wherein said peptide comprises an amino acid sequence that shares at least 96% identity with SEQ ID NO:3.

14. The composition of claim 13, wherein said amino acid sequence shares at least 97% identity with SEQ ID NO:3.

15. The composition of claim 13, wherein said amino acid sequence shares at least 98% identity with SEQ ID NO:3.

16. A composition comprising an isolated nucleic acid, wherein said nucleic acid comprises SEQ ID NO:1 or the complement of SEQ ID NO:1.

17. A composition comprising an isolated peptide, wherein said peptide comprises SEQ ID NO:3.

18. A method for determining the risk of eye disease comprising:
a) providing nucleic acid from a subject, wherein said nucleic acid comprises an LPH gene; and
b) detecting the presence or absence of at least one variation in said LPH gene.

19. The method of claim 18, further comprising step c) providing a diagnosis to said subject based on the presence or absence of said variation.

20. The method of claim 18, wherein said variation is a mutation.

21. The method of claim 18, wherein said variation is a polymorphism.

22. The method of claim 18, wherein said LPH gene is selected from human LPH1, human LPH2, and human LPH3.

23. The method of claim 18, wherein said LPH gene is a human LPH3 sequence, and said variation prevents the peptide encoded by said human LPH3 sequence from binding the human TIGR peptide.
